(19) United States
(12) Patent Application Publication
(10) Pub. No.: US 2025/0327063 A1
(43) Pub. Date: Oct. 23, 2025

(54) PREPARATION OF LONG READ NUCLEIC ACID LIBRARIES

(71) Applicant: Illumina, Inc., San Diego, CA (US)

(72) Inventors: Heather Meinholz, Cottage Grove, WI (US); Daniel Gerhardt, Madison, WI (US); Joshua Burgess, Sun Prairie, WI (US); Leigh Graham Monahan, Stanmore (AU); Jennifer Dennin, Mt. Horeb, WI (US); Stephen Paul Bruinsma, Madison, WI (US); M. Maggie O'Meara, Madison, WI (US); Aaron Earl Darling, Hornsby (AU); Johnathan Parker, Dane, WI (US); Stewart MacArthur, Cambridge (GB); Denise Raterman, Madison, WI (US); Feng Chen, Castro Valley, CA (US); Susan C. Verity, Cambridge (GB); Esther Musgrave-Brown, Cambridge (GB); Andrew Slatter, Cambridge (GB); Tina Huang, Los Altos, CA (US); Tiffany Truong, San Francisco, CA (US); Yoon Hye Shin, Chula Vista, CA (US); David Tse, San Diego, CA (US)

(21) Appl. No.: 18/867,223

(22) PCT Filed: May 25, 2023
(86) PCT No.: PCT/US2023/067466

§ 371 Date: Nov. 19, 2024

Related U.S. Application Data
(60) Provisional application No. 63/365,361, filed on May 26, 2022.
(60) Provisional application No. 63/366,222, filed on Jun. 10, 2022.
(60) Provisional application No. 63/366,516, filed on Jun. 16, 2022.
(60) Provisional application No. 63/366,896, filed on Jun. 23, 2022.
(60) Provisional application No. 63/373,685, filed on Aug. 26, 2022.
(60) Provisional application No. 63/483,213, filed on Feb. 3, 2023.

(51) Int. Cl. C12N 15/10; C12Q 1/6806; C12Q 1/6853; C12Q 1/686; C12Q 1/6874

(52) U.S. Cl. CPC C12N 15/1031; C12N 15/1093; C12Q 1/6806; C12Q 1/6853; C12Q 1/686; C12Q 1/6874

(57) ABSTRACT

Some embodiments of the methods and compositions provided herein relate to obtaining long read information from short reads of a target nucleic acid. Some embodiments include steps to selectively generate, mark, and amplify long nucleic acid fragments. Some embodiments include enriching for certain sequences in the long fragments with selection probes directed to certain challenging medically relevant genes (CMRG). Some embodiments also include fragmenting the long nucleic acid fragments into shorter fragments for sequencing, and informatically reconstructing a sequence of the target nucleic acid.

### RELATED APPLICATIONS

This application claims priority to U.S. Prov. App. No. 63/483,213 filed Feb. 3, 2023; U.S. Prov. App. No. 63/373,685 filed Aug. 26, 2022; U.S. Prov. App. No. 63/366,896 filed Jun. 23, 2022; U.S. Prov. App. No. 63/366,516 filed Jun. 16, 2022; U.S. Prov. App. No. 63/366,222 filed Jun. 10, 2022; and U.S. Prov. App. No. 63/365,361 filed May 26, 2022, which are each entitled “PREPARATION OF LONG READ NUCLEIC ACID LIBRARIES” and which are each incorporated by reference herein in its entirety.

### REFERENCE TO SEQUENCE LISTING

The present application is being filed along with a Sequence Listing in electronic format. The Sequence Listing is provided as a file entitled ILLINC736WO3, created May 20, 2023, which is approximately 189,995,442 bytes in size. The information in the electronic format of the Sequence Listing is incorporated herein by reference in its entirety.

### FIELD OF THE INVENTION

Some embodiments of the methods and compositions provided herein relate to obtaining long read information from short reads of a target nucleic acid. Some embodiments include steps to selectively generate, mark, and amplify long nucleic acid fragments. Some embodiments include enriching for certain sequences in the long fragments with selection probes directed to certain challenging medically relevant genes (CMRG). Some embodiments also include fragmenting the long nucleic acid fragments into shorter fragments for sequencing, and informatically reconstructing a sequence of the target nucleic acid.

### BACKGROUND OF THE INVENTION

Current protocols for next-generation sequencing (NGS) of nucleic acid samples routinely employ a sample preparation process that converts DNA or RNA into a library of fragmented, sequenceable templates. Sample preparation methods often require multiple steps, material transfers, and expensive instruments to effect fragmentation, and therefore are often difficult, tedious, expensive, and inefficient.

In one approach, nucleic acid fragment libraries may be prepared using a transposome-based method where two transposon end sequences, one linked to a tag sequence, and a transposase form a transposome complex. The transposome complexes are used to fragment and tag target nucleic acids in solution to generate a sequencer-ready tagmented library. The transposome complexes may be immobilized on a solid surface, such as through a biotin molecule appended at the 5′ end of one of the two end sequences. Use of immobilized transposomes can provide advantages over solution-phase approaches by reducing hands-on and overall library preparation time, cost, and reagent requirements, lowering sample input requirements, and enabling the use of unpurified or degraded samples as a starting point for library preparation. However, certain portions of a genome may be underrepresented in libraries prepared using such transposomes.

### SUMMARY OF THE INVENTION

Some embodiments of the methods and compositions provided herein a method for preparing a nucleic acid library, comprising: (a) obtaining a plurality of transposomes comprising transposon adaptors, wherein the plurality of transposomes is immobilized on a solid support; (b) contacting a plurality of nucleic acid fragments with the plurality of transposomes to obtain a plurality of polynucleotides; (c) amplifying the plurality of polynucleotides to obtain amplified polynucleotides; and (d) adding library adapters to each end of the amplified polynucleotides, thereby obtaining the nucleic acid library.

In some embodiments, the solid support comprises a bead. In some embodiments, the plurality of the transposomes is immobilized on the bead at a density such that an average length of the plurality of polynucleotides is greater than about 1 kbp, 2 kbp, 5 kbp, 10 kbp, 15 kbp, 20 kbp, or 40 kbp; and/or wherein the average length of the plurality of polynucleotides is in a range from about 1 kbp to about 40 kbp, 1 kbp to about 30 kbp, 1 kbp to about 20 kbp, 5 kbp to about 20 kbp, 5 kbp to about 15 kbp, or 7 kbp to about 12 kbp. In some embodiments, the number of transposomes immobilized on the bead is no more than about 100 transposomes, 50 transposomes, 40 transposomes, 30 transposomes, 20 transposomes, or 10 transposomes. In some embodiments, the number of transposomes immobilized on the bead is no more than about 30 transposomes. In some embodiments, the plurality of the transposomes immobilized on the bead comprise a total activity such that an average length of the plurality of polynucleotides greater than about 1 kbp, 2 kbp, 5 kbp, 10 kbp, 15 kbp, 20 kbp, or 40 kbp; and/or wherein the average length of the plurality of polynucleotides is in a range from about 1 kbp to about 40 kbp, 1 kbp to about 30 kbp, 1 kbp to about 20 kbp, 5 kbp to about 20 kbp, 5 kbp to about 15 kbp, or 7 kbp to about 12 kbp. In some embodiments, the plurality of the transposomes immobilized on the bead comprise an activity in a range from about 0.05 AU/μl to about 0.25 0.05 AU/μl. In some embodiments, the plurality of the transposomes immobilized on the bead comprise an activity of about 0.075 AU/μl. In some embodiments, the transposon adapters comprise the same sequence. In some embodiments, the transposomes of the plurality of transposomes are the same. In some embodiments, the transposomes of the plurality of transposomes are B15 transposomes. In some embodiments, the transposon adapters comprise the nucleotide sequence: SEQ ID NO:01 (GTCTCGTGGGCTCGG).

In some embodiments, the step (c) comprises a mutagenesis PCR, such that mutations are introduced into amplified polynucleotides. In some embodiments, the mutagenesis PCR comprises amplifying the plurality of polynucleotides with a low bias DNA polymerase, and/or with a nucleotide analogue. In some embodiments, the nucleotide analogue comprises dPTP, and/or S-oxo-dGTP. In some embodiments, the low bias DNA polymerase is a Thermococcal polymerase, or a functional derivative thereof. In some embodiments, the Thermococcal polymerase is derived from a Thermococcal strain selected from the group consisting of T. kodakarensis, T. siculi, T. celer and T. sp KS-1. In some embodiments, the mutagenesis PCR comprises no more than 12 cycles, 10 cycles, 9 cycles, S cycles, 7 cycles, 6 cycles, 5 cycles, 4 cycles, 3 cycles, or 2 cycles. In some embodiments, the mutagenesis PCR comprises no more than 6 cycles.

In some embodiments, a first end of a polynucleotide of the plurality of polynucleotides is capable of annealing to a second end of the polynucleotide of the plurality of polynucleotides; and/or, wherein a first end of an amplified polynucleotide is capable of annealing to a second end of the amplified polynucleotide. In some embodiments, step (c) further comprises a suppression PCR in some embodiments, the suppression PCR comprises use of a single amplification primer. In some embodiments, the amplified polynucleotides have an average length greater than about 1 kbp, 2 kbp, 3 kbp, 4 kbp, 5 kbp, 10 kbp, 15 kbp, or 20 kbp. In some embodiments, the suppression PCR comprises no more than 16 cycles, 14 cycles, 10 cycles, 9 cycles, S cycles, 7 cycles, 6 cycles, 5 cycles, 4 cycles, 3 cycles, or 2 cycles. In some embodiments, the suppression PCR comprises no more than 6 cycles.

Some embodiments also include enriching for target nucleic acids in the amplified polynucleotides. Some embodiments also include enriching for target nucleic acids in the plurality of polynucleotides. In some embodiments, the enriching for target nucleic acids in the amplified polynucleotides is performed after performing the mutagenesis PCR, and before performing the suppression PCR. In some embodiments, the enriching for target nucleic acids in the amplified polynucleotides is performed after performing the suppression PCR. Some embodiments also include amplifying the target nucleic acids.

In some embodiments, step (d) comprises contacting the amplified polynucleotides with an additional plurality of transposomes comprising the library adapters. In some embodiments, the library adapters comprise (i) indexes, (ii) bridge amplification primer binding sites, and/or (iii) sequencing primer binding sites. Some embodiments also include enriching for target polynucleotides in the nucleic acid library.

In some embodiments, the enriching comprises hybridizing a plurality of selection probes with the amplified polynucleotides, the plurality of polynucleotides, and/or the nucleic acid library, wherein the selection probes of the plurality of selection probes comprise different nucleotide sequences from one another. In some embodiments, an average distance between two adjacent nucleotide sequences of the selection probes on a reference sequence of a genome is in a range from about 300 consecutive nucleotides to about 7,000 consecutive nucleotides; optionally, wherein the range is from about 500 consecutive nucleotides to about 5,000 consecutive nucleotides; optionally, wherein the range is from about 750 consecutive nucleotides to about 2,500 consecutive nucleotides; optionally, wherein the range is from about 750 consecutive nucleotides to about 1,500 consecutive nucleotides; and optionally, wherein the range is from about 900 consecutive nucleotides to about 1,200 consecutive nucleotides. In some embodiments, an average distance between two adjacent nucleotide sequences of the selection probes on a reference sequence of a genome is about 750, 1000, 1500, or 2000 consecutive nucleotides. In some embodiments, an average number of sites in a genome that each selection probe of the plurality of selection probes is capable of hybridizing to is no more than 50 different sites in the genome, to no more than 40 different sites in the genome, to no more than 30 different sites in the genome, to no more than 20 different sites in the genome. In some embodiments, each selection probe of the plurality of selection probes is capable of hybridizing to no more than 50 different sites in a genome, to no more than 40 different sites in a genome, to no more than 30 different sites in a genome, to no more than 20 different sites in a genome. In some embodiments, a selection probe capable of hybridizing to a site in the genome comprises at least 50, 60, 70, or 80 consecutive nucleotides complementary to at least 90% of a nucleotide sequence at the site in the genome.

In some embodiments, the plurality of selection probes comprise at least 50, 100, 200, 500, 1000, 5000 different selection probes. In some embodiments, each selection probe of the plurality of selection probes comprises a nucleotide sequence capable of hybridizing to a gene selected from TABLE 1C; and/or a nucleotide sequence within, no more than 10 kbp 5′ or no more than 10 kbp 3′ of a gene selected from TABLE 1C. In some embodiments, each selection probe of the plurality of selection probes comprises a nucleotide sequence having at least 90%, 95%, or 100% sequence identity to any one of SEQ ID NOs:02-140600. In some embodiments, each selection probe of the plurality of selection probes comprises a nucleotide sequence having at least 90%, 95%, or 100% sequence identity to any one of SEQ ID NOs:02-43879. In some embodiments, each selection probe of the plurality of selection probes comprises a nucleotide sequence having at least 90%, 95%, or 100% sequence identity to any one of SEQ ID NOs:02-22546. In some embodiments, the plurality of selection probes is attached to a substrate. In some embodiments, the substrate comprises a plurality of beads; optionally wherein the beads are magnetic.

Some embodiments also include amplifying the target polynucleotides. In some embodiments, an amount of the plurality of nucleic acid fragments is less than about 100 ng, 50 ng, 30 ng, 20 ng, 10 ng, 5 ng, or 1 ng. In some embodiments, the plurality of nucleic acid fragments is mammalian. In some embodiments, the plurality of nucleic acid fragments is human. In some embodiments, a plurality of nucleic acid fragments comprises genomic DNA.

Some embodiments of the methods and compositions provided herein a method for preparing a nucleic acid library, comprising: (a) obtaining a plurality of transposomes comprising transposon adaptors, wherein the plurality of transposomes is immobilized on a bead, wherein the transposomes of the plurality of transposomes are the same; (b) contacting a plurality of nucleic acid fragments with the plurality of transposomes to obtain a plurality of polynucleotides, wherein the plurality of the transposomes immobilized on the bead comprise a total activity such that an average length of the plurality of polynucleotides greater than about 1 kbp, 2 kbp, 5 kbp, 10 kbp, 15 kbp, 20 kbp, or 40 kbp; (c) amplifying the plurality of polynucleotides to obtain amplified polynucleotides by: (i) performing a mutagenesis PCR, such that mutations are introduced into amplified polynucleotides, and (ii) performing a suppression PCR; and (d) adding library adapters to each end of the amplified polynucleotides by contacting the amplified polynucleotides with an additional plurality of transposomes, thereby obtaining the nucleic acid library.

Some embodiments also include enriching for target nucleic acids in the amplified polynucleotides, and/or enriching for target nucleic acids in the nucleic acid library. In some embodiments, enriching for target nucleic acids in the amplified polynucleotides is performed prior to performing the suppression PCR. In some embodiments, enriching for target nucleic acids in the amplified polynucleotides is performed after performing the suppression PCR. In some embodiments, the enriching comprises hybridizing a plurality of selection probes with the amplified polynucleotides and/or the nucleic acid library.

In some embodiments, the enriching comprises hybridizing a plurality of selection probes with the amplified polynucleotides, the plurality of polynucleotides, and/or the nucleic acid library, wherein the selection probes of the plurality of selection probes comprise different nucleotide sequences from one another. In some embodiments, an average distance between two adjacent nucleotide sequences of the selection probes on a reference sequence of a genome is in a range from about 300 consecutive nucleotides to about 7,000 consecutive nucleotides; optionally, wherein the range is from about 500 consecutive nucleotides to about 5,000 consecutive nucleotides; optionally, wherein the range is from about 750 consecutive nucleotides to about 2,500 consecutive nucleotides; optionally, wherein the range is from about 750 consecutive nucleotides to about 1,500 consecutive nucleotides; and optionally, wherein the range is from about 900 consecutive nucleotides to about 1,200 consecutive nucleotides. In some embodiments, an average distance between two adjacent nucleotide sequences of the selection probes on a reference sequence of a genome is about 750, 1000, 1500, or 2000 consecutive nucleotides. In some embodiments, an average number of sites in a genome that each selection probe of the plurality of selection probes is capable of hybridizing to is no more than 50 different sites in the genome, to no more than 40 different sites in the genome, to no more than 30 different sites in the genome, to no more than 20 different sites in the genome. In some embodiments, each selection probe of the plurality of selection probes is capable of hybridizing to no more than 50 different sites in a genome, to no more than 40 different sites in a genome, to no more than 30 different sites in a genome, to no more than 20 different sites in a genome. In some embodiments, a selection probe capable of hybridizing to a site in the genome comprises at least 50, 60, 70, or 80 consecutive nucleotides complementary to at least 90% of a nucleotide sequence at the site in the genome.

In some embodiments, the plurality of selection probes comprise at least 50, 100, 200, 500, 1000, 5000 different selection probes. In some embodiments, each selection probe of the plurality of selection probes comprises a nucleotide sequence capable of hybridizing to a gene selected from TABLE 1C; and/or a nucleotide sequence within, no more than 10 kbp 5′ or no more than 10 kbp 3′ of a gene selected from TABLE 1C. In some embodiments, each selection probe of the plurality of selection probes comprises a nucleotide sequence having at least 90%, 95%, or 100% sequence identity to any one of SEQ ID NOs:02-140600. In some embodiments, each selection probe of the plurality of selection probes comprises a nucleotide sequence having at least 90%, 95%, or 100% sequence identity to any one of SEQ ID NOs:02-43879. In some embodiments, each selection probe of the plurality of selection probes comprises a nucleotide sequence having at least 90%, 95%, or 100% sequence identity to any one of SEQ ID NOs:02-22546. In some embodiments, the plurality of selection probes is attached to a substrate; optionally, wherein the substrate comprises a plurality of beads; optionally wherein the beads are magnetic.

Some embodiments of the methods and compositions provided herein a method for determining a sequence of a target nucleic acid, comprising: performing any one of the foregoing methods; sequencing the nucleic acid library to obtain sequence reads; and assembling sequence reads to obtain the sequence of a target nucleic acid. In some embodiments, the assembling comprises comparing the sequence reads to a reference sequence. In some embodiments, the comparing comprises determining mutations introduced into the amplified polynucleotides during the mutagenesis PCR. In some embodiments, the reference sequence is obtained from the same nucleic acid sample as the plurality of nucleic acid fragments.

Some embodiments of the methods and compositions provided herein a kit comprising: a first bead-linked transposomes (BIT-1) reagent, wherein the BLT-1 transposomes comprises a first adaptor sequence; a mutagenesis reagent comprising a first primer, dPTPs, dNTPs, and a polymerase; a second bead-linked transposomes (BLT-2) reagent, wherein the BLT-2 transposomes comprise the first adaptor and a second adaptor; an amplification reagent comprising a first primer, a second primer, dNTPs, and a polymerase; wherein BLT-1 has a lower transposome density as compared to BLT-2; and wherein the first primer hybridizes to the first adaptor sequence and the second primer hybridizes to the second adaptor sequence. In some embodiments, BLT-2 has more than 10, 20, 50, 100, or 1000 times the transposome density as compared to BLT-1. In some embodiments, the first adaptor is B15 and the second adaptor is A14.

Some embodiments of the methods and compositions provided herein a system for preparing a nucleic acid library, comprising: (a) a first plurality of transposomes comprising transposon adaptors for tagmenting a plurality of nucleic acid fragments, wherein the first plurality of transposomes is immobilized on a first plurality of beads at a first density; (b) reagents for amplifying the plurality of polynucleotides to obtain amplified polynucleotides, wherein the amplifying comprising a mutagenesis PCR and/or a suppression PCR, wherein: (i) the first reagent for performing mutagenesis PCR comprise a low bias DNA polymerase and/or a nucleotide analogue; optionally, wherein the nucleotide analogue comprises dPTP, and/or 8-oxo-dGTP; and/or the low bias DNA polymerase is a Thermococcal polymerase, or a functional derivative thereof, optionally, wherein the Thermococcal polymerase is derived from a Thermococcal strain selected from the group consisting of T. kodakarensis, T. siculi, T. celer and T. sp. KS-1, and (ii) the first reagents for performing suppression PCR comprise amplification primers having the same nucleotide sequence capable of hybridizing to the transposon adaptors; (c) a plurality of selection probes for enriching for target polynucleotides in the amplified polynucleotides; and (d) a second plurality of transposomes comprising library adaptors for adding library adaptors to each end of the amplified polynucleotides, wherein the second plurality of transposomes is immobilized on a second plurality of beads at a second density, wherein the first density is less than the second density.

Some embodiments of the methods and compositions provided herein a system for preparing a nucleic acid library, comprising: (a) a first plurality of transposomes for tagmenting a plurality of nucleic acid fragments to obtain a plurality of polynucleotides, wherein the first plurality of transposomes comprises transposon adaptors, wherein the first plurality of transposomes is immobilized on a solid support, optionally, wherein the solid support comprises a first plurality of beads; wherein: the first plurality of the transposomes is immobilized on the first plurality of beads at a density such that on contacting the first plurality of transposomes with the plurality of nucleic acid fragments the plurality of polynucleotides has an average length of the plurality of polynucleotides is greater than about 1 kbp, 2 kbp, 5 kbp, 10 kbp, 15 kbp, 20 kbp, or 40 kbp; and/or wherein the average length of the plurality of polynucleotides is in a range from about 1 kbp to about 40 kbp, 1 kbp to about 30 kbp, 1 kbp to about 20 kbp, 5 kbp to about 20 kbp, 5 kbp to about 15 kbp, or 7 kbp to about 12 kbp; the number of transposomes immobilized on the bead is no more than about 100 transposomes, 50 transposomes, 40 transposomes, 30 transposomes, 20 transposomes, or 10 transposomes, optionally, wherein the number of transposomes immobilized on the bead is no more than about 30 transposomes; the plurality of the transposomes immobilized on the bead comprise a total activity such that on contacting the first plurality of transposomes with the plurality of nucleic acid fragments the plurality of polynucleotides has an average length greater than about 1 kbp, 2 kbp, 5 kbp, 10 kbp, 15 kbp, 20 kbp, or 40 kbp; and/or wherein the average length of the plurality of polynucleotides is in a range from about 1 kbp to about 40 kbp, 1 kbp to about 30 kbp, 1 kbp to about 20 kbp, 5 kbp to about 20 kbp, 5 kbp to about 15 kbp, or 7 kbp to about 12 kbp; and/or the plurality of the transposomes immobilized on the bead comprise an activity in a range from about 0.05 AU/μl to about 0.25 0.05 AU/μl, optionally, wherein the plurality of the transposomes immobilized on the bead comprise an activity of about 0.075 AU/μL; (b) first reagents for amplifying the plurality of polynucleotides to obtain amplified polynucleotides; and (c) second reagents for adding library adaptors to each end of the amplified polynucleotides. In some embodiments, the transposon adapters comprise the same sequence, optionally, wherein the transposon adapters comprise the nucleotide sequence: SEQ ID NO:01 (GTCTCGTGGGCTCGG); and/or wherein the transposomes of the plurality of transposomes are the same, optionally, wherein the transposomes of the plurality of transposomes are B15 transposomes.

In some embodiments, the first reagents comprise reagents for performing mutagenesis PCR comprising a low bias DNA polymerase and/or a nucleotide analogue; optionally, wherein: the nucleotide analogue comprises dPTP, and/or 8-oxo-dGTP; and/or the low bias DNA polymerase is a Thermococcal polymerase, or a functional derivative thereof, optionally, wherein the Thermococcal polymerase is derived from a Thermococcal strain selected from the group consisting of T. kodakarensis, T. siculi, T. celer and T. sp KS-1. In some embodiments, the first reagents comprise reagents for performing suppression PCR comprising amplification primers having the same nucleotide sequence; optionally, wherein the amplification primers are capable of hybridizing to the transposon adaptors.

In some embodiments, the second reagents comprise a second plurality of transposomes comprising the library adaptors; and optionally, wherein the second plurality of transposomes has an activity such that on contacting the second plurality of transposomes with the amplified polynucleotides a library of nucleic acids is obtained and comprises the library adaptors and having an average length less than about 1 kb, 900 bp, 800, bp, 700 bp, 600 bp, 500 bp, 400 bp, 300 bp, 200 bp, or 100 bp. In some embodiments, the first plurality of the transposomes is immobilized on the beads at a density less than a density at which the second plurality of transposomes are immobilized on the second plurality of beads.

Some embodiments also include third reagents for enriching for target polynucleotides in the amplified polynucleotides, comprising a plurality of selection probes; optionally, wherein the plurality of selection probes is attached to a third plurality of beads. In some embodiments, an average distance between two adjacent nucleotide sequences of the selection probes on a reference sequence of a genome is in a range from about 300 consecutive nucleotides to about 7,000 consecutive nucleotides; optionally, wherein the range is from about 500 consecutive nucleotides to about 5,000 consecutive nucleotides; optionally, wherein the range is from about 750 consecutive nucleotides to about 2,500 consecutive nucleotides; optionally, wherein the range is from about 750 consecutive nucleotides to about 1,500 consecutive nucleotides; and optionally, wherein the range is from about 900 consecutive nucleotides to about 1,200 consecutive nucleotides; and optionally, wherein an average distance between two adjacent nucleotide sequences of the selection probes on a reference sequence of a genome is about 750, 1000, 1500, or 2000 consecutive nucleotides. In some embodiments, an average number of sites in a genome that each selection probe of the plurality of selection probes is capable of hybridizing to is no more than 50 different sites in the genome, to no more than 40 different sites in the genome, to no more than 30 different sites in the genome, to no more than 20 different sites in the genome. In some embodiments, each selection probe of the plurality of selection probes is capable of hybridizing to no more than 50 different sites in a genome, to no more than 40 different sites in a genome, to no more than 30 different sites in a genome, to no more than 20 different sites in a genome; and optionally, wherein a selection probe capable of hybridizing to a site in the genome comprises at least 50, 60, 70, or 80 consecutive nucleotides complementary to at least 90% of a nucleotide sequence at the site in the genome. In some embodiments, the plurality of selection probes comprise at least 50, 100, 200, 500, 1000, 5000 different selection probes. In some embodiments, each selection probe of the plurality of selection probes comprises a nucleotide sequence capable of hybridizing to a gene selected from TABLE 1C; and/or a nucleotide sequence within, no more than 10 kbp 5′ or no more than 10 kbp 3′ of a gene selected from TABLE 1C. In some embodiments, each selection probe of the plurality of selection probes comprises a nucleotide sequence having at least 90%, 95%, or 100% sequence identity to any one of SEQ ID NOs:02-140600. In some embodiments, each selection probe of the plurality of selection probes comprises a nucleotide sequence having at least 90%, 95%, or 100% sequence identity to any one of SEQ ID NOs:02-43879. In some embodiments, each selection probe of the plurality of selection probes comprises a nucleotide sequence having at least 90%, 95%, or 100% sequence identity to any one of SEQ ID NOs:02-22546.

In some embodiments, the plurality of nucleic acid fragments is mammalian. In some embodiments, the plurality of nucleic acid fragments is human. In some embodiments, the plurality of nucleic acid fragments comprises genomic DNA.

Some embodiments of the methods and compositions provided herein a kit comprising: a plurality oat least 50, 100, 1000, 2000, 3000, 4000, 5000, 10000, 20000, 30000, or 40000 selection probes, wherein the selection probes are different from one another, and comprise a nucleotide sequence having at least 90%, 95%, or 100% sequence identity to any one of SEQ ID NOs:02-140600; and optionally: (i) a first plurality of transposomes comprising transposon adaptors for tagmenting a plurality of nucleic acid fragments, wherein the first plurality of transposomes is immobilized on a first plurality of beads at a first density; and (ii) a second plurality of transposomes comprising library adaptors for adding library adaptors to each end of the amplified polynucleotides, wherein the second plurality of transposomes is immobilized on a second plurality of beads at a second density, wherein the first density is less than the second density. In some embodiments, each selection probe of the plurality of selection probes comprises a nucleotide sequence having at least 90%, 95%, or 100% sequence identity to any one of SEQ ID NOs:02-43879, or to any one of SEQ ID NOs:02-22546.

### BRIEF DESCRIPTION OF THE DRAWINGS

FIG. 1 depicts an example embodiment of a workflow which includes: fragmenting long input DNA by high molecular weight (HMW) fragmentation and adding adapters, such as by tagmentation using low density bead linked transposomes (BLTs); long range PCR mutagenesis to introduce a signature into long fragments; further library preparation steps, such as additional tagmentation to obtain small fragments with adapters; sequencing and assembly of sequencing reads.

FIG. 2 depicts an example embodiment of a workflow which includes a long-read (iLR) pathway, and a reference pathway. The long-read pathway includes steps for: tagmentation; mutagenesis; bottlenecking (suppression) PCR. Both the long-read pathway and reference pathway share steps including: standard library preparation, such as tagmentation; sequencing; and assembly of sequencing reads,

FIG. 3A is a graph which relates to a purified bottlenecking PCR product run on an Agilent Bioanalyzer using a High Sensitivity DNA Kit.

FIG. 3B is a graph which relates to a purified final library prep product run on an Agilent Bioanalyzer using a High Sensitivity DNA Kit.

FIG. 4A depicts a graph of results using transposomes in solution at various concentration

FIG. 4B depicts a graph of results for size distribution using BLTs.

FIG. 4C depicts graphs for a Staphylococcus aureus 4 Mb genome view, with samples at 4 million reads, GC content: 32.9%, size: 0.8 Mb. A: Nextera XT (soluble transposomes); B: Flex (BLT): 1 ng; C: Flex (BLT): 100 ng, D: Flex (BLT): colony direct.

FIG. 5 depicts a schematic for workflow steps including HMW fragmentation; and mutagenesis and suppression PCR in which smaller products form hairpins.

FIGS. 6A-6C depict graphs related to activity and fragment length. FIG. 6A is a graph of actual activity units (AU)/μl and median AU/μl versus build AU/μl for soluble transposomes (TSM), and BLTs having various densities/activities of transposomes: BLT at low density (BLT-LR) at 0.075 AU/μl, and TDER-BLT comprising A14 and B15 TSMs at 0.1 AU/μl, 0.2 AU/μl, and 0.5 AU/μl. In FIG. 6A, “TDER-BLR” is “TDER-BLT”. FIG. 6B is a graph of fragment size. FIG. 6C is a graph for average size for soluble TSM, and BLTs containing A14 and B15 TSMs, or B15 TSM only.

FIGS. 7A-7C depict graphs related to mutagenesis PCR for soluble TSM, and BLTs containing A14 and B15 TSMs, or B15 TSM only. FIG. 7A is a graph of mean yield (ng/μl). FIG. 7B is a graph for average size. FIG. 7C is a graph for mean average size.

FIGS. 8A-8C depict graphs related to bottleneck (suppression) PCR for soluble TSM, and BLTs containing A14 and B15 TSMs, or B15 TSM only. FIG. 8A is a graph of mean yield (ng/μl). FIG. 8B is a graph for size distribution. FIG. 8C is a graph for mean average size.

FIG. 9 depicts a graph for a sequencing metric (GC coverage) for soluble TSM, and BLTs containing A14 and B15 TSMs, or B15 TSM only.

FIGS. 10A and 10B depict graphs for a N50 sequencing metric for soluble TSM, and BLTs containing A14 and B15 TSMs, or B15 TSM only. N50 is the length of the shortest contig for which longer and equal length contigs cover at least 50% of the assembly. FIG. 10A depicts a graph for N50. FIG. 10B depicts a graph for N50 by regions.

FIG. 11 depicts a graph for a sequencing metric (fraction of bases with no coverage, left panel; and fraction of bases with <10× coverage, right panel) for soluble TSM, and BLTs containing A14 and B15 TSMs, or B15 TSM only.

FIG. 12 depict graphs of various BLT activities (Build AU/μl), and product average size (lower panel), total yield (middle panel), or fluorescent resonance energy transfer (FRET) (upper panel).

FIG. 13 depicts graphs of various BLT activities (Build AU/μl), and sequencing metrics including SLR coverage depth (lower panels), total bases (middle panels), or N50 (upper panels).

FIG. 14 depicts graphs of various BLT activities (Build AU/μl), and sequencing metrics including percent duplicated reads (lower panels), fraction of bases with <10× coverage (middle panels), or fraction of bases with no coverage (upper panels).

FIG. 15 depicts graphs of various BLT activities (AU/μl), and sequencing metrics including SLR coverage depth (lower panel), total bases (lower middle panel), redundancy (upper middle panel), or N50 (upper panel) with three different operators.

FIG. 16 depicts graphs of tagmentation yield (left panel) or tagmentation fragment length (right panel) for various amounts of input DNA.

FIG. 17 depicts graphs for various amounts of input DNA and mutagenesis yield (upper left panel), bottleneck yield (middle left panel), library yield (lower left panel), mutagenesis fragment length (upper right panel), bottleneck fragment length (middle right panel), and library fragment length (lower right panel).

FIG. 18 depicts graphs for various amounts of input DNA and sequencing metrics including: total bases (upper left panel), insert size (middle left panel), percent duplicated reads (lower left panel), total bases (upper right panel), insert size (middle right panel), and library fragment length (lower right panel). The right panels show the same data as the left panels, but without the 1000 ng data point.

FIG. 19 depicts graphs for various amounts of input DNA and sequencing metrics including: number of MQ0 reads (upper left panel), error rate (upper middle left panel), redundancy (lower middle left panel), N50 (lower left panel), number of MQ0 reads (upper right panel), error rate (upper middle right panel), redundancy (lower middle right panel), N50 (lower right panel). The right panels show the same data as the left panels, but without the 1000 ng data point.

FIG. 20 depicts graphs for various amounts of input DNA and sequencing metrics including: mode coverage (tipper left panel), fraction of bases with no coverage (middle left panel), fraction of bases with <1 OX coverage (lower left panel), mode coverage (upper right panel), fraction of bases with no coverage (middle right panel), fraction of bases with <1 OX coverage (lower right panel). The right panels show the same data as the left panels, but without the 1000 ng data point.

FIG. 21 depicts a graph for various amounts of input DNA and sequencing metric (GC bias).

FIG. 22 depicts graphs for various input DNAs subjected to shearing for different periods of time, control input DNA, and HMW input DNA, and fragment size.

FIG. 23 depicts graphs for various input DNAs subjected to shearing for different periods of time, control input DNA, and HMW input DNA, and tagmentation yield (left panel) or tagmentation fragment length (right panel).

FIG. 24A depicts graphs for various input DNAs subjected to shearing for different periods of time, control input DNA, and HMW input DNA, and mutagenesis yield (left panel) or normalization yield (right panel).

FIG. 24B depicts graphs for various input DNAs subjected to shearing for different periods of time, control input DNA, and HMW input DNA, and bottleneck PCR yield (left panel) or post-bottleneck fragment length (right panel).

FIG. 25 depicts graphs for various input DNAs subjected to shearing for different periods of time, and HMW input DNA, and sequencing metrics: N50 (left panels) or redundancy (right panels).

FIG. 26 depicts graphs for various input DNAs subjected to shearing for different periods of time, and HMW input DNA, and sequencing metrics: SLR coverage (upper left panel), fraction with no coverage (middle left panel), fraction with <10× coverage (lower left panel), insert size (upper right panel), percent duplicated reads (upper middle right panel), insertion per 100 kb (lower middle right panel), or MQ0 (lower right panel).

FIG. 27 depicts graphs for various input DNAs subjected to shearing for different periods of time, and HMW input DNA, and a sequencing metric (GC bias).

FIG. 28 depicts an example overview for enrichment of ‘long fragments’ or ‘short fragments’ in a workflow.

FIG. 29 depicts an example timeline for enrichment of ‘long fragments’ or ‘short fragments’ in a workflow.

FIG. 30 depicts selection of probes with higher specificity in long fragments.

FIG. 31 depicts design of probes in regions adjacent to problematic regions of the genome.

FIG. 32 depicts embodiments including an example work flow for whole genome sequencing (WGS) with optional enrichment steps, and parallel standard short read (SR) library preparation.

FIG. 33 depicts use of 80mer probes with long fragments in problematic target regions, compared to use of probes with short fragments (upper panel); use of 80mer probes with flanked long difficult regions, compared to use of probes with short fragments (middle panel); and use of 80mer probes with long fragments in regions with infrequent probe coverage, compared to use of probes with short fragments (lower panel).

FIG. 34 depicts results of sequencing coverage for a targeted region using a method with long fragments and enrichment (ICLR with enrichment).

FIG. 35 depicts results of sequencing coverage for a targeted a 722 kb region in the MHC locus using a method with long fragments and enrichment.

FIG. 36 depicts results of sequencing coverage for a targeted a 426 kb region in the MHC locus that covered HLA-A, HLA-G, HLA-F using a method with long fragments and enrichment.

FIG. 37A depicts a graph for SNV precision and recall for methods which included (i) on market long reads; (ii) PCR free (tagmentation to provide short fragments); (iii) long fragments with enrichment using an MHC selection probe panel (ICLR-MHC enrichment); and (iv) long fragments without enrichment (ICLR-WGS).

FIG. 37B depicts a graph for Indel precision and recall for methods which included (i) on market long reads; (ii) PCR free (tagmentation to provide short fragments); (iii) long fragments with enrichment using an MHC selection probe panel (ICLR-MHC enrichment); and (iv) long fragments without enrichment (ICLR-WGS).

FIG. 38 depicts a graph for coverage in ACM genes using methods which included long fragments with enrichment using an ACMG selection probe panel (enrichment) or long fragments without enrichment (WGS).

FIG. 39 depicts results of sequencing coverage for a TNNT2, a 22 kb gene which was fully phased in one phase block, using a method with long fragments and enrichment with an ACMG panel of selection probes.

FIG. 40A depicts results of sequencing coverage for APOB which was fully phased in one phase block, using a method with long fragments and enrichment with an A CMG panel of selection probes.

FIG. 40B depicts results of sequencing coverage for TMEM127 which was fully phased in one phase block, using a method with long fragments and enrichment with an ACMG panel of selection probes.

FIG. 41 depicts results of sequencing coverage for MSH6 using a method with long fragments and enrichment with an ACMG panel of selection probes (ICLR with enrichment), and a method with long fragments without enrichment (ICLR WGS).

FIG. 42A depicts a graph for SNV precision and recall for methods which included (i) on market long reads; (ii) PCR free (tagmentation to provide short fragments); (iii) long fragments with enrichment using an ACMG selection probe panel (ICLR-ACMG enrichment); and (iv) long fragments without enrichment (ICLR-WGS).

FIG. 42B depicts a graph for Indel precision and recall for methods which included (i) on market long reads; (ii) PCR free (tagmentation to provide short fragments); (iii) long fragments with enrichment using an ACMG selection probe panel (ICLR-ACMG enrichment); and (iv) long fragments without enrichment (ICLR-WGS).

FIG. 43A depicts a graph for SNV precision and recall for methods which included (i) on market long reads; (ii) PCR free (tagmentation to provide short fragments); (iii) long fragments with enrichment using a PGX selection probe panel (ICLR-PGX enrichment); and (iv) long fragments without enrichment (ICLR-WGS).

FIG. 43B depicts a graph for Indel precision and recall for methods which included (i) on market long reads; (ii) PCR free (tagmentation to provide short fragments); (iii) long fragments with enrichment using a PGX selection probe panel (ICLR-PGX enrichment); and (iv) long fragments without enrichment (ICLR-WGS).

FIG. 44A depicts three graphs comparing use of a first selection probe panel (SYD-C2-CMRG-230 bp) and a second selection probe panel (SYD-C2-CMRG-1 kb) for (i) total mutations in bases in region (upper left panel); (ii) percentage DUP mutant reads (upper right panel); and (iii) percentage on target unique mapped reads (lower panel).

FIG. 44B depicts a graph of normalized coverage between use of a first selection probe panel (SYD-C2-CMRG-230 bp) and a second selection probe panel (SYD-C2-CMRG-1 kb).

FIG. 44C depicts a comparison between results of sequencing coverage for HBG1 using a method (i) with long fragments and enrichment with an CMRG panel of selection probes (ICLR with enrichment); (ii) with long fragments without enrichment (ICLR WGS); and (iii) tagmentation with short reads (PCR free short read).

FIG. 45A depicts a graph for SNV precision and recall for methods which included (i) on market long reads; (ii) PCR free (tagmentation to provide short fragments); (iii) long fragments with enrichment using a CMRG selection probe panel (ICLR-CMRG enrichment); and (iv) long fragments without enrichment (ICLR-WGS).

FIG. 45B depicts a graph for Indel precision and recall for methods which included (i) on market long reads; (ii) PCR free (tagmentation to provide short fragments); (iii) long fragments with enrichment using a CMRG selection probe panel (ICLR-CMRG enrichment); and (iv) long fragments without enrichment (ICLR-WGS).

### DETAILED DESCRIPTION

Some embodiments of the methods and compositions provided herein relate to obtaining long read information from short reads of a target nucleic acid. Some embodiments include steps to selectively generate, mark, and amplify long nucleic acid fragments. Some embodiments include enriching for certain sequences in the long fragments with selection probes directed to challenging medically relevant genes (CMRG). Some embodiments also include fragmenting the long nucleic acid fragments into shorter fragments for sequencing, and informatically reconstructing a sequence of the target nucleic acid.

Prior fragmentation methods typically generated a very wide distribution of fragment sizes such that even when aiming for large fragments, inevitably short fragments were included. Such short fragments are ‘wasted’ space, giving very little new information. Some embodiments provided herein preserve long (about 2,000-40,000 bp) fragments, mark them, and carry them through into a short-read portion of a workflow so they can then be reconstructed into their parent long fragments informatically. Shorter fragments are generally much less desirable, and may take up valuable sequencing space and informatics volume if they are included. Use of long fragments enables the use of a smaller number of selection probes to enrich for target sequences in the long fragments.

In prior short read library preps, most size selection was done by a combination of (1) initial fragmentation and (2) Solid-Phase Reversible Immobilization (SPRI)-based size selection. However, SPRI size selection primarily works on fragments smaller than about 60 Gbp in length. In contrast, suppression (“bottlenecking” or “bottleneck”) PCR acts on larger fragments. Suppression PCR entails appending complementary sequences on 5′ and 3′ ends of the same DNA molecule, such that during a PCR annealing step, there is a direct competition between annealing of a primer and annealing of opposite ends of the same DNA fragment. When the PCR primer anneals, extension proceeds as normal, and the fragment is amplified. When opposite ends anneal, for example by forming a hairpin, there is no templated 3′ hydroxyl to extend, and so amplification does not occur. A key to suppression PCR and size selection is that for shorter fragments, the opposite ends of the same fragment are closer together and therefore more likely to find each other and anneal. Under optimized conditions, this leads to preferential amplification of longer fragments. Aspects of suppression PCR useful with embodiments provided herein are described in Dai, Z-M, et al (2006) J. of Biotech 128:435-443; and Rand K. N. et al. (2005) N.A. Res. 33:e127 which are incorporated by reference in their entireties.

In some embodiments provided herein, complementary 5′ and 3′ ends are achieved by an initial tagmentation step with B15 transposomes only. Typically, tagmentation would be performed with a combination of A14 and B15 transposomes so that the different sequences can be used for read 1 and read 2 primers during subsequent sequencing. However, because the initial tagmentation in certain embodiments provided herein is used to provide a landing spot for PCR, different sequences for read 1 and read 2 primers do not need to be added at this stage. In contrast to SPRI size selection, it was observed that by adding cycles of suppression PCR, the number of smaller fragments tinder 2000 bp in length can be dramatically reduced.

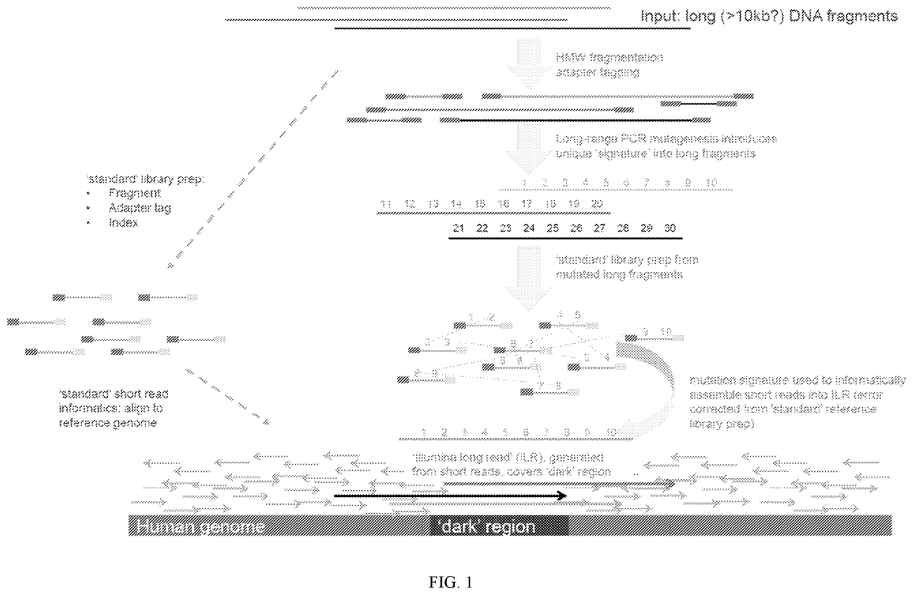

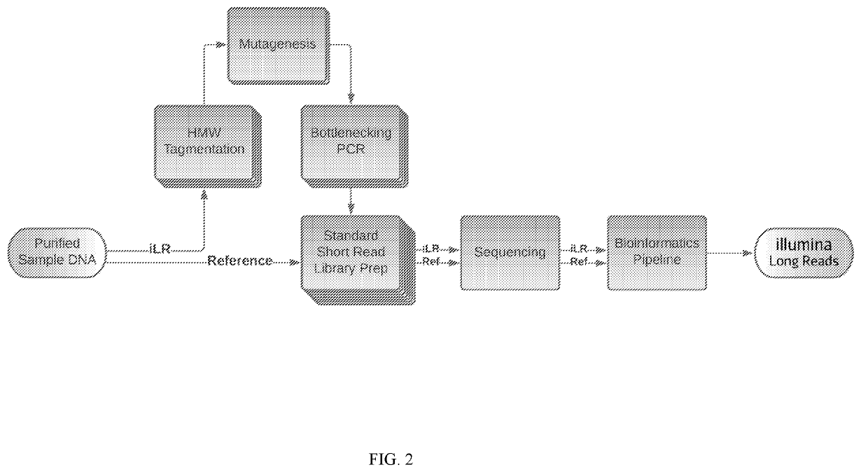

In some embodiments provided herein a workflow includes: fragmenting long input DNA by high molecular weight (HMW) fragmentation and adding adapters, such as by tagmentation using low density bead linked transposomes (BLTs); long range PCR mutagenesis to introduce a signature into long fragments; further library preparation steps, such as additional tagmentation to obtain small fragments with adapters; sequencing and assembly of sequencing reads (FIG. 1). In some embodiments provided herein a workflow includes a long-read (“iLR” or “ILR”) pathway, and a reference pathway. The long-read pathway includes steps for: tagmentation; mutagenesis; bottlenecking (suppression) PCR. Both the long-read pathway and reference pathway share steps including: standard library preparation, such as tagmentation; sequencing; and assembly of sequencing reads (FIG. 2).

Certain aspects useful with embodiments of the methods and compositions provided herein are disclosed in U.S. Pat. Nos. 9,040,256; 9,683,230; and U.S. 2021/0010008 which are each incorporated by reference in its entirety.

### Definitions

As used herein, the term “nucleic acid” refers to a polynucleotide sequence, or fragment thereof. A nucleic acid can comprise nucleotides. A nucleic acid can be exogenous or endogenous to a cell. A nucleic acid can exist in a cell-free environment. A nucleic acid can be a gene or fragment thereof. A nucleic acid can be DNA. A nucleic acid can be RNA. A nucleic acid can comprise one or more analogs (e.g., altered backbone, sugar, or nucleobase). Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. “Nucleic acid”, “polynucleotide, “target polynucleotide, and “target nucleic acid” can be used interchangeably. As used herein, “kbp” can refer to kilobase pairs and relates to a length of a double-stranded nucleic acid. The length of a nucleic acid may also be referred to in terms of a number of nucleotides, such as consecutive nucleotides.

As used herein “transposome” includes a complex comprising of at least one transposase enzyme and a transposon recognition sequence, such as a transposon adapter. In some such systems, the transposase binds to a transposon recognition sequence to form a functional complex that is capable of catalyzing a transposition reaction. In some aspects, the transposon recognition sequence is a double-stranded transposon end sequence. The transposase, or integrase, binds to a transposase recognition site in a target nucleic acid and inserts the transposon recognition sequence into a target nucleic acid. In some such insertion events, one strand of the transposon recognition sequence (or end sequence) is transferred into the target nucleic acid, resulting also in a cleavage event. Exemplary transposition procedures and systems that can be readily adapted for use with the transposases of the present disclosure are described, for example, in WO10/048605, U.S. 2012/0301925, U.S. 2012/13470087, or U.S. 2013/0143774, each of which is incorporated herein by reference in its entirety.

In some embodiments, the transposome complex is a dimer of two molecules of a transposase. In some embodiments, the transposome complex is a homodimer, wherein two molecules of a transposase are each bound to first and second transposons of the same type (e.g., the sequences of the two transposons bound to each monomer are the same, forming a “homodimer”). In some embodiments, the compositions and methods described herein employ two populations of transposome complexes. In some embodiments, the transposases in each population are the same. In some embodiments, the transposome complexes in each population are homodimers, wherein the first population has a first adaptor sequence in each monomer and the second population has a different adaptor sequence in each monomer.

As used herein “solid surface,” “solid support,” and other grammatical equivalents refer to any material that is appropriate for or can be modified to be appropriate for the attachment of the transposome complexes. As will be appreciated by those in the art, the number of possible substrates is multitude. Possible substrates include, but are not limited to, glass and modified or functionalized glass, plastics (including acrylics, polystyrene and copolymers of styrene and other materials, polypropylene, polyethylene, polybutylene, polyurethanes, TEFLON, etc.), polysaccharides, nylon or nitrocellulose, ceramics, resins, silica or silica-based materials including silicon and modified silicon, carbon, metals, inorganic glasses, plastics, optical fiber bundles, beads, paramagnetic beads, and a variety of other polymers. In some such embodiments, the transposome complex is immobilized on the solid support via the linker. In some further embodiments, the solid support comprises or is a tube, a well of a plate, a slide, a bead, or a flowcell, or a combination thereof. In some further embodiment, the solid support comprises or is a bead. In one embodiment, the bead is a paramagnetic bead. In some of the methods and compositions presented herein, transposome complexes are immobilized to a solid support. In one embodiment, the solid support is a bead. Suitable bead compositions include, but are not limited to, plastics, ceramics, glass, polystyrene, methylstyrene, acrylic polymers, paramagnetic materials, thoria sol, carbon graphite, titanium dioxide, latex or cross-linked dextrans such as Sepharose, cellulose, nylon, cross-linked micelles and TEFLON, as well as any other materials outlined herein for solid supports.

As used herein, “tagmentation: includes to the modification of DNA by a transposome complex comprising transposase enzyme complexed with adaptors comprising transposon end sequence. Tagmentation results in the simultaneous fragmentation of the DNA and ligation of the adaptors to the 5′ ends of both strands of duplex fragments. Following a purification step to remove the transposase enzyme, additional sequences can be added to the ends of the adapted fragments, for example by PCR, ligation, or any other suitable methodology known to those of skill in the art.

### Certain Methods for Preparing Nucleic Acid Libraries

Some embodiments of the methods and compositions providing herein include preparing a nucleic acid library. Some such embodiments include (a) obtaining a plurality of transposomes comprising transposon adaptors, wherein the plurality of transposomes is immobilized on a solid support; (b) contacting a plurality of nucleic acid fragments with the plurality of transposomes to obtain a plurality of polynucleotides; (c) amplifying the plurality of polynucleotides to obtain amplified polynucleotides; and (d) adding library adapters to each end of the amplified polynucleotides, thereby obtaining the nucleic acid library. In some embodiments, an amount of the plurality of nucleic acid fragments is less than about 100 ng, 50 ng, 30 ng, 20 ng, 10 ng, 5 ng, or 1 ng.

Some embodiments include an initial tagmentation step which fragments the plurality of nucleic acids fragments and adds an adaptor to each end of the products of the tagmentation. The initial tagmentation is limited such that the products of the tagmentation are longer than a tagmentation where the activity of transposomes is not limited.

Certain aspects useful with embodiments of the methods and compositions provided herein are disclosed in U.S. Pat. Nos. 9,115,396; 9,080,211; 9,040,256; U.S. patent application publication 2014/0194324, each of which is incorporated herein by reference in its entirety.

In some embodiments, the solid support comprises a bead. In some such embodiments, the transposomes are bead-linked transposomes (BLTs). In some embodiments, the activity of the transposomes on the beads is such that a tagmentation reaction with the BLTs and the plurality of nucleic acid fragments results in long polynucleotides, such as polynucleotides an having average length of the plurality of polynucleotides greater than about 1 kbp, 2 kbp, 5 kbp, 10 kbp, 15 kbp, 20 kbp, or 40 kbp; and/or wherein the average length of the plurality of polynucleotides is in a range from about 1 kbp to about 40 kbp, 1 kbp to about 30 kbp, 1 kbp to about 20 kbp, 5 kbp to about 20 kbp, 5 kbp to about 15 kbp, or 7 kbp to about 12 kbp. For example, the transposomes can be bound at a low density on the beads; and/or have a low tagmentation activity. In some embodiments, the number of transposomes immobilized on the bead is no more than about 100 transposomes, 50 transposomes, 40 transposomes, 30 transposomes, 20 transposomes, or 10 transposomes. In some embodiments, the number of transposomes immobilized on the bead is no more than about 30 transposomes. In some embodiments, the plurality of the transposomes immobilized on the bead comprise a total activity such that an average length of the plurality of polynucleotides greater than about 1 kbp, 2 kbp, 5 kbp, 10 kbp, 15 kbp, 20 kbp, or 40 kbp; and/or wherein the average length of the plurality of polynucleotides is in a range from about 1 kbp to about 40 kbp, 1 kbp to about 30 kbp, 1 kbp to about 20 kbp, 5 kbp to about 20 kbp, 5 kbp to about 15 kbp, or 7 kbp to about 12 kbp. In some embodiments, the plurality of the transposomes immobilized on the bead comprise a tagmentation activity in a range from about 0.05 AU/μl to about 0.25 AU/μl. In some embodiments, the plurality of the transposomes immobilized on the bead comprise a tagmentation activity of about 0.075 AU/μl.

In some embodiments, the transposomes on the beads are the same. For example, in some embodiments, the transposon adapters comprise the same sequence. In some embodiments, the transposomes of the plurality of transposomes are B15 transposomes. In some embodiments, the transposon adapters comprise the nucleotide sequence: SEQ ID NO:01 (GTCTCGTGGGCTCGG).

Some embodiments also include steps to add a signature to the products of the initial tagmentation. For example, a signature can be added into the sequence of the library products by steps that include limited mutagenesis. In some embodiments, step (c) comprises a mutagenesis PCR, such that mutations are introduced into amplified polynucleotides. In some embodiments, the mutagenesis PCR comprises amplifying the plurality of polynucleotides with a low bias DNA polymerase, and/or with a nucleotide analogue. In some embodiments, the nucleotide analogue comprises dPTP (such as, 6H,8H=3,4-Dihydro-pyrimido(4,5-c)(1,2)oxazin-7-one-8-β-D-2-deoxy-ribofuranoside-5′-triphosphate), and/or 8-oxo-dG-TP. dP contains the bicyclic pyrimidine analog 3,4-dihydro-8H-pyrimido-[4,5-C][1,2]oxazin-7-one. In some embodiments, the low bias DNA polymerase is a Thermococcal polymerase, or a functional derivative thereof. In some embodiments, the Thermococcal polymerase is derived from a Thermococcal strain selected from the group consisting of T. kodakarensis, T. siculi, celer and T. sp KS-1. In some embodiments, the mutagenesis PCR comprises no more than 12 cycles, 10 cycles, 9 cycles, 8 cycles, 7 cycles, 6 cycles, 5 cycles, 4 cycles, 3 cycles, or 2 cycles. In some embodiments, the mutagenesis PCR comprises no more than 6 cycles.

Some embodiments also include a bottlenecking or suppression PCR step to enrich for longer polynucleotides. For example, shorter amplified polynucleotides form hairpins, while longer amplified polynucleotides may be further amplified. In other words, the bottlenecking or suppression PCR can be biased against the amplification of shorter nucleic acids in a mixture of nucleic acids of different lengths. Some such embodiments can enrich for longer fragments. In some such embodiments, a first end of a polynucleotide of the plurality of polynucleotides is capable of annealing to a second end of the polynucleotide of the plurality of polynucleotides; and/or, wherein a first end of an amplified polynucleotide is capable of annealing to a second end of the amplified polynucleotide. In some embodiments, the suppression PCR comprises use of a single amplification primer. In some embodiments, the amplified polynucleotides have an average length greater than about 1 kbp, 2 kbp, 3 kbp, 4 kbp, 5 kbp, 10 kbp, 15 kbp, or 20 kbp. In some embodiments, the suppression PCR comprises no more than 16 cycles, 14 cycles, 10 cycles, 9 cycles, 8 cycles, 7 cycles, 6 cycles, 5 cycles, 4 cycles, 3 cycles, or 2 cycles. In some embodiments, the suppression PCR comprises no more than 6 cycles.

Detailed descriptions of certain embodiments of suppression PCR are found in, e.g., U.S. Pat. No. 5,565,340 and Siebert et al., Nucleic Acids Res., 23(6):1087-1088 (1995). Briefly, the inverted repeat sequences function as suppression tails by competing with the suppression PCR primer for complementary binding. The inverted repeats tend to anneal each other, thereby preventing PCR primer binding. Since shorter amplicons undergo inverted repeat annealing more often than longer amplicons, the suppression PCR favors generating long amplicons.

Some embodiments also include enriching for target nucleic acids in the amplified polynucleotides, such as products of the suppression PCR. In some embodiments, the enriching comprises hybridizing a plurality of selection probes with the amplified polynucleotides. In some embodiments, the plurality of selection probes lack sequences capable of hybridizing to a repetitive genomic DNA element. In some embodiments, the repetitive genomic DNA element is selected from a tandem repeat, an Alu repeat, a short interspersed nuclear element (SINE), a long interspersed nuclear element (LINE), an integrated viral sequence, a viral long terminal repeat (LTR), and a transposon. Some embodiments also include amplifying the target nucleic acids.

Some embodiments also include preparing a library of shorter fragments from the products of the suppression PCR, and/or the enrichment. For example, the products of the suppression PCR, and/or the enrichment can undergo an additional tagmentation. In some embodiments, step (d) comprises contacting the amplified polynucleotides with an additional plurality of transposomes. In some embodiments, the additional plurality of transposomes comprise transposon adapters comprising (i) indexes, (ii) bridge amplification primer binding sites, and/or (iii) sequencing primer binding sites. An example of a bridge amplification primer binding site includes a sequence capable of binding a capture probe on a surface, wherein the capture probe comprises a primer extended during bridge amplification on the surface.

Some embodiments also include enriching for target polynucleotides in the library of nucleic acids. In some embodiments, the enriching comprises hybridizing a plurality of selection probes with the library of nucleic acids, wherein the plurality of selection probes is capable of specifically hybridizing with the target polynucleotides. Some embodiments also include amplifying the target polynucleotides.

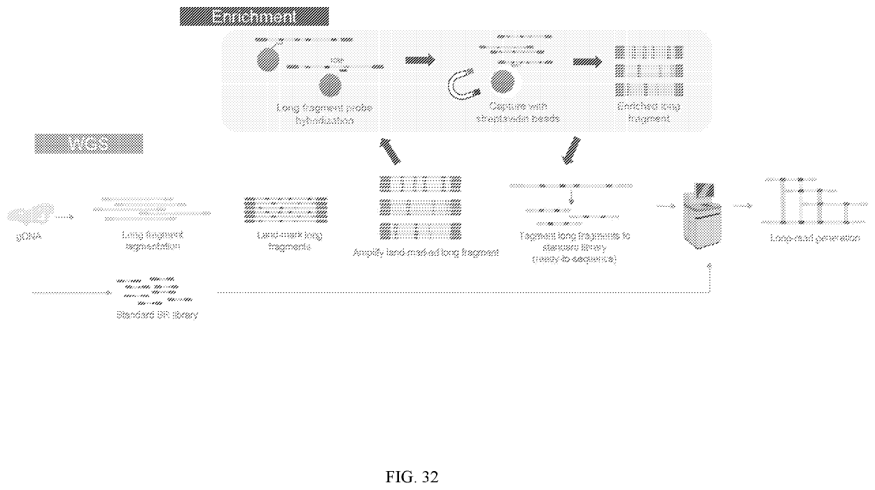

Some embodiments include methods for preparing a nucleic acid library, comprising: (a) obtaining a plurality of transposomes comprising transposon adaptors, wherein the plurality of transposomes is immobilized on a bead, and wherein the transposomes of the plurality of transposomes are the same; (b) contacting a plurality of nucleic acid fragments with the plurality of transposomes to obtain a plurality of polynucleotides, wherein the plurality of the transposomes immobilized on the bead comprise a total activity such that an average length of the plurality of polynucleotides greater than about 1 kbp, 2 kbp, 5 kbp, 10 kbp, 15 kbp, 20 kbp, or 40 kbp; and/or wherein the average length of the plurality of polynucleotides is in a range from about 1 kbp to about 40 kbp, 1 kbp to about 30 kbp, 1 kbp to about 20 kbp, 5 kbp to about 20 kbp, 5 kbp to about 15 kbp, or 7 kbp to about 12 kbp; (c) amplifying the plurality of polynucleotides to obtain amplified polynucleotides by: (i) performing a mutagenesis PCR, such that mutations are introduced into amplified polynucleotides, and (ii) performing a suppression PCR; and (d) adding library adapters to each end of the amplified polynucleotides by contacting the amplified polynucleotides with an additional plurality of transposomes, thereby obtaining the nucleic acid library. An example embodiment of a workflow is depicted in FIG. 32 which includes whole genome sequencing (WGS) which includes: fragmenting genomic DNA into long fragments by limited tagmentation; land-marking or adding a signature to the long fragments; amplifying the long fragments with a bias against shorter fragments; tagmenting products prior to sequencing. Optional steps include enrichment of the amplified long fragments with a panel of selection probes, such as capture probes with certain sequences. A parallel workflow includes tagmenting the genomic DNA to form a library of short fragments, such as a standard short read (SR) library, and sequencing the library of short fragments.

Some embodiments also include enriching for target nucleic acids in the amplified polynucleotides. In some embodiments, the enriching comprises hybridizing a plurality of selection probes with the amplified polynucleotides, wherein the plurality of selection probes is capable of specifically hybridizing with the target nucleic acids. In some embodiments, the plurality of selection probes lack sequences capable of hybridizing to a repetitive genomic DNA element. In some embodiments, the repetitive genomic DNA element is selected from a tandem repeat, an Alu repeat, a short interspersed nuclear element (SINE), a long interspersed nuclear element (LINE), an integrated viral sequence, a viral long terminal repeat (LTR), and a transposon. Some embodiments also include amplifying the target nucleic acids.

Some embodiments also include enriching for target polynucleotides in the library of nucleic acids. In some embodiments, the enriching comprises hybridizing a plurality of selection probes with the library of nucleic acids, wherein the plurality of selection probes is capable of specifically hybridizing with the target polynucleotides. Some embodiments also include amplifying the target polynucleotides.

Some embodiments also include methods for determining a sequence of a target nucleic acid, comprising preparing a nucleic acid library by any one of the embodiments above, sequencing the library of nucleic acids to obtain sequence reads; and assembling sequence reads to obtain the sequence of a target nucleic acid. In some embodiments, the assembling comprises comparing the sequence reads to a reference sequence. In some embodiments, the reference sequence is obtained from the same nucleic acid sample as the plurality of nucleic acid fragments.

### Target Enrichment

Certain embodiments provided herein include enriching for target nucleic acids during preparation of a nucleic acid library, such as certain challenging medically relevant genes (CMRG). FIG. 32 shows an example work flow which includes steps for whole genome sequencing (WGS) with additional enrichment steps. In some embodiments, the nucleic acid library is prepared from a nucleic acids sample with bead-linked transposomes to generate long tagmented polynucleotides. The polynucleotides are marked with methods, such as mutagenesis amplification. In an optional step, suppression/bottlenecking PCR is performed where amplification is biased against shorter self-annealing nucleic acids. Target nucleic acids can then be enriched for in the amplified nucleic acids using selection probes. Additional steps can include a further tagmentation step to generate shorter library nucleic, sequencing the library nucleic acids, and comparing the generated sequence with a reference sequence obtained from the same nucleic acid sample.

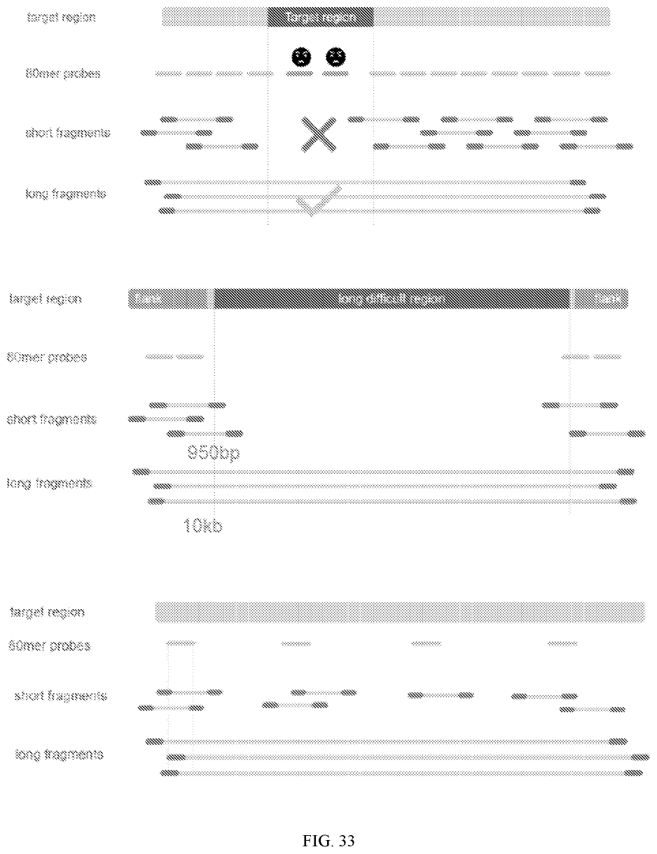

Enrichment efficiency is increased compared to conventional library preparation methods because hybridization of selection probes is to long tagmented polynucleotides. For example, specificity, such as percentage hybridization to target, directly affects the amount of sequencing needed to achieve target coverage depth. High specificity hybridization to target can be difficult to achieve in long repetitive regions and pseudogenes. However, long fragment hybridization has several advantages over short fragment. As shown in FIG. 33, long fragment hybridization allows removal of ineffective probes; allows strategic placement of probes, such as adjacent to problematic genomic regions; and allows the use of fewer probes to cover a region previously covered by many probes. Targeted enrichments can achieve cost-effective human whole genome coverage. For example, genomic regions that may have been typically underrepresented in conventional nucleic acid libraries can be targeted with selection probes. Some embodiments provided herein include the use of certain panels of selection probes, such panels a Major Histocompatibility Complex (MHC) panel directed to the MHC region; an American College of Medical Genetics and Genomics (ACMG) panel directed to genes for which specific mutations are known to be causative of disorders that are clinically actionable; a pharmacogenetic (PGX) panel directed to genes commonly targeted by pharmacogenetic testing assays; a challenging medically relevant gene (CMRG) panel directed to medically relevant autosomal genes that may be under-represented in certain tests due to repeats or polymorphic complexities; and a comprehensive genome wide (dark) panel directed to genomic regions typically underrepresented in sequencing reads.

### Major Histocompatibility Complex (MC) Panel

Some embodiments provided herein relate to targeting the MHC region of the human genome. Some such embodiments include generation and/or use of a selection probe panel to target the MHC region. The MHC region is a large locus located on the short arm of human chromosome 6 (6p21.1-6p21.3), and contains highly polymorphic genes that code for cell surface proteins essential for the adaptive immune system. The region is challenging to obtain sequence information due to the presence of a high level of repetitive sequences, sequence homology, pseudogenes, and a wide variety of alleles in the population. Precise genotyping and phasing of the MHC region is challenging but highly clinically desirable for applications such as organ transplantation and drug discovery.

### American College of Medical Genetics (ACMG) Panel

Some embodiments provided herein relate to targeting a panel of genes, such as an American College of Medical Genetics (ACMG) panel of genes. Some such embodiments include generation and/or use of a selection probe panel to target the ACMG genes. A selection probe panel was generated from a list of genes compiled by ACMG for which specific mutations are known to be causative of disorders that are clinically actionable. Miller D. T., et al., Genet Med. 2022 Jul.; 24(7):1407-1414. doi: 10.1016/j.gim.2022.04.006.

A selection probe panel was designed to precisely call variants and phase in these genes. The panel included 78 unique genes in ACMG SF v3.1, and targeted full genes. The panel size was about 6.8 Mbp. The ACMG genes include those listed in TABLE 1A.

TABLE 1A
ACMG genes

ACTA2, ACTC1, ACVRL1, APC, APOB, ATP7B, BAG3, BMPR1A, BRCA1, BRCA2,
BTD, CACNA1S, CASQ2, COL3A1, DES, DSC2, DSG2, DSP, ENG, FBN1, FLNC, GAA,
GLA, HFE, HNF1A, KCNH2, KCNQ1, LDLR, LMNA, MAX, MEN1, MLH1, MSH2,
MSH6, MUTYH, MYBPC3, MYH11, MYH7, MYL2, MYL3, NF2, OTC, PALB2, PCSK9,
PKP2, PMS2, PRKAG2, PTEN, RB1, RBM20, RET, RPE65, RYR1, RYR2, SCN5A,
SDHAF2, SDHB, SDHC, SDHD, SMAD3, SMAD4, STK11, TGFBR1, TGFBR2,
TMEM127, TMEM43, TNNC1, TNNI3, TNNT2, TP53, TPM1, TRDN, TSC1, TSC2, TTN,
TTR, VHL, WT1

### Pharmacogenetic (PGX) Panel

Some embodiments provided herein relate to targeting a panel of genes such as a panel of pharmacogenetic (PGX) genes. Some such embodiments include generation and/or use of a selection probe panel to target the PGX genes. A selection probe panel was generated for genes commonly targeted by pharmacogenetic testing assays. Kalman L. V. et al., Clin Pharmacol Ther. 2016 February; 99(2): 172-185. doi:10.1002/cpt.280. Genetic variation is known to influence the way individual respond to therapeutics. Accurately detecting functional haplotypes, such as haplotypes associated with protein activity levels (“star alleles”) in clinically actionable pharmacogenetic genes is crucial to implementation of personalized medicine. The panel was generated to achieve highly accurate genotyping and star allele calling in such genes. The panel included 98 genes that are important in pharmacogenetics, targeting full genes. The panel size was about 8.1 Mbp. The genes include those listed in TABLE 1B.

TABLE 1B
PGX genes

ABCB1, ABCG2, ABL1, ACE, ADH1A, ADH1B, ADH1C, ADRA2A, ADRB1, ADRB2,
AHR, ALDH1A1, ALK, ALOX5, ANKK1, ASL, BCHE, BCR, BRAF, BRCA1,
CACNA1S, CFTR, COMT, CYP1A1, CYP1A2, CYP2A13, CYP2A6, CYP2B6,
CYP2C18, CYP2C19, CYP2C8, CYP2C9, CYP2D6, CYP2E1, CYP2J2, CYP3A4,
CYP3A5, CYP3A7, CYP4F2, DPYD, DRD2, EGFR, ERBB2, F2, F5, G6PD, GRIK4,
GRK4, GRK5, GSTM1, GSTP1, HLA-A, HLA-B, HLA-C, HLA-DPB1, HLA-DQA1,
HLA-DQB1, HLA-DRA, HLA-DRB1, HMGCR, HTR2A, HTR2C, IFNL3, IFNL4,
KCNH2, KCNJ11, KIT, KRAS, MTHFR, NAT1, NAT2, NQO1, NR1I2, NRAS, NUDT15,
OPRM1, P2RY1, P2RY12, PTGIS, PTGS2, RYR1, SCN5A, SLC15A2, SLC19A1,
SLC22A1, SLC22A2, SLCO1B1, SLCO2B1, SULT1A1, TPMT, TYMS, UGT1A1,
UGT1A4, UGT2B15, UGT2B17, UGT2B7, VDR, VKORC1

### Challenging Medically Relevant Gene (CMRG) Panel

Some embodiments provided herein relate to targeting a panel of genes such as a panel of challenging medically relevant genes (CMRG). Some such embodiments include generation and/or use of a selection probe panel to target the CMRG. The repetitive nature and complexity of some medically relevant genes poses a challenge for their accurate analysis in a clinical setting (Wagner J., et al, (2022) Nat Biotechnol. 40:672-680). The Genome in a Bottle (GIAB) Consortium has provided variant benchmark sets, but these exclude nearly four hundred medically relevant genes due to their repetitiveness or polymorphic complexity. Multiple regions of the genome are not fully resolved in existing benchmarks due to repetitive sequence, segmental duplications, and complex variants, such as multiple nearby SNVs, INDELs, and/or SVs. Goldfeder R L et al. Genome Med. 8, 1-12 (2016).

About 5175 gene symbols have been previously identified from the following sources: (1) 4,773 potentially medically-relevant genes from the databases OMIM, HGMD, and ClinVar which includes both commonly tested and rarely tested genes (Mandelker D et al. Genet. Med 18, 1282-1289 (2016). (2) The COSMIC gene census contains 723 gene symbols found in tumors (Tate J G et al. Nucleic Acids Res. 47, D941-D947 (2019)), (3) A focused list of “High Priority Clinical Genes” that are commonly tested for clinical inherited diseases (Wagner J., et al., (2022) Nat Biotechnol. 40:672-680).

5,027 have unique coordinates on the primary assembly of GRCh38 and valid ENSEMBL annotations, and 4,697 are autosomal. 70% of these genes are specific to the list from OMIM, HGMD, and ClinVar, which includes genes associated with disease in a small number of studies and are currently tested more frequently in research studies than in high-throughput clinical laboratories. 395 genes included 90% or less on GRCh37 or GRCh38. Many of the 395 medically relevant genes were not covered well by the v4.2.1 small variant benchmark due to SVs, complex variants, and segmental duplications. A CMRG benchmark for 273 of the 395 genes has been previously created. (Wagner J., et al., (2022) Nat Biotechnol. 40:672-680). To be included in the CMRG benchmark, the entire gene including 20 kb flanking sequence on each side and any overlapping segmental duplications needed to have exactly one fully aligned contig from each haplotype with no breaks on GRCh37 and GRCh38. The total size of 391 of the CMRG gene panel is about 22.5 Mbp. The smallest gene is about 66 bp (SNORD64), the largest gene is about 1 Mbp (PTPRN2). CMRG genes include those listed in TABLE 1C.

TABLE 1C
CMRG genes

A4GALT, ABCG8, ABO, ABR, ADAMTS10, ADAMTSL2, AFP, AGL, AGRN,
ALOXE3, ANKRD11, ANO7, APOBEC1, APOBEC3H, APOC1, APOC2, APOC4,
ARHGEF10, ASIP, ATPAF2, AXIN1, B3GAT3, BAX, BFSP2, BLOC1S3, BRAF, BSG,
BTRC, C1R, C3, CABIN1, CALR3, CANT1, CASP10, CBR3, CBS, CCL3L1, CD247,
CD320, CD4, CD55, CDH15, CDH17, CEL, CFC1, CFC1B, CFD, CFHR1, CFHR3, CHL1,
CHMP1A, CHRNA4, CLCN7, CLIP2, CNR2, COL18A1, COL6A1, COL6A2, COX14,
COX6B1, CR1, CREB3L3, CRYAA, CTDP1, CYB5R3, CYP2G1P, CYP4F12, CYP4F3,
D2HGDH, DAXX, DAZL, DCLRE1C, DEAF1, DGCR6, DIP2C, DLGAP2, DMPK,
DNAAF4, DNMT3L, DOK7, DPP6, DPY19L2, DRD4, DSPP, DUX4, DUX4L1, ECHS1,
EEF1A2, EHMT1, EIF2B5, EIF4E, ELANE, ELOA3, ENO3, ESPN, ESRRA, ETFB,
ETHE1, EXTL2, F7, FAM20C, FAT1, FCGR1A, FCGR2B, FCGR3A, FGF3, FGFRL1,
FKBP8, FLAD1, FLG, FLT4, FOXN1, FSCN2, FTCD, FUT1, FUT3, FXN, G6PC3, GAK,
GALNT9, GALR1, GALT, GCGR, GCSH, GDF3, GIP, GIPC3, GNPTG, GOLGA3,
GP1BA, GP6, GPI, GPIHBP1, GRIN1, GRK1, GSTM1, GTF2I, GTF2IRD2, GUSB,
GYPA, GYPB, GYPE, H19, HBG1, HBM, HCN2, HCN3, HEATR2, HES7, HLA-B, HLA-
DQB1, HLA-DRB1, HMGCL, HMX1, HNF1A, HOMER2, HOXB8, HPD, HSD11B2,
HYAL1, HYDIN, IFITM3, IFNL3, IGHA1, IGHG1, IGHG2, IGHM, IGHV3-21, IGKC,
IGKV1-5, IKBKB, IKZF1, IMPA1, INPP5D, INPP5E, INSL3, INSR, JAG2, KANSL1,
KATNAL2, KCNE1, KCNJ18, KCNV2, KDM2B, KIR2DL1, KIR2DL3, KIR3DL1,
KISS1, KISS1R, KLF11, KLF14, KLK4, KMT2C, KNG1, KRTAP1-1, LAMB1, LBR,
LCE3B, LHFPL5, LIPN, LIX1, LMF1, LMNB2, LPA, LRIG2, LRPAP1, LZTFL1, MAFA,
MAN1B1, MAP2K3, MARVELD2, MASP2, MBOAT7, MC1R, MDK, MEST, MLC1,
MLPH, MOGS, MPG, MRC1, MST1R, MUC1, MUC16, MUC3A, MUC4, MUC5B,
MUSK, MYO9B, MYOT, MYT1, NACA, NAIP, NAPRT, NBEAP1, NCF1, NCF1C,
NCR3, NDUFA6, NDUFAF1, NDUFB1, NDUFV3, NFKBIL1, NLRP12, NLRP2, NLRP7,
NOD1, NOTCH2, NPM1, NPPA, NSMF, NUTM2B, NUTM2D, OCLN, OPRL1, OR12D2,
OR4F5, OR51A2, ORC6, P2RX2, P2RX5, PADI4, PAPSS2, PCBP1, PCCB, PCDHA10,
PCMT1, PDE4DIP, PDE6B, PDLIM3, PDPK1, PDSS1, PEX5, PGAM5, PGAP6, PHKG2,
PIGV, PKD1, PKN3, PLA2G10, PLTP, PMS2, PNKP, POLG2, PPIA, PPIP5K1, PRG4,
PRKCG, PRODH, PROZ, PRSS2, PSPH, PTEN, PTK6, PTPRC, PTPRN2, PTPRQ, PXDN,
RFX2, RGPD3, RHCE, RHOA, RNF212, RNF213, RPIA, RPL22, RPN1, RPS17,
RXYLT1, SAR1B, SBK3, SDHA, SEC63, SEMG1, SERPINF2, SH2B1, SHANK2,
SHANK3, SIGLEC16, SIRT3, SLC17A5, SLC22A1, SLC22A12, SLC26A9, SLC27A4,
SLC27A5, SLC29A4, SLC5A11, SLC6A18, SLC6A3, SMG1, SMN1, SMN2, SMOC2,
SNORD64, SNTG2, SOHLH1, SPATA31C1, SPI1, SPRN, SRGAP2, SRR, SSTR5,
STK11, STXBP2, SULT1A1, SUZ12, TAPBP, TAS2R46, TBXA2R, TCF3, TERT, TFPT,
THBS2, TJP2, TM4SF19, TMC6, TMEM114, TNNI3, TNNT1, TNNT3, TPCN2, TPO,
TRAPPC10, TRBV9, TRMT1, TRPM4, TTC37, TTLL1, TUBGCP6, TWIST2, TYK2,
TYMS, U2AF1, UGT2A1, UGT2A2, UGT2B17, UGT2B28, UNKL, USP8, UTP4,
UVSSA, VANGL1, VKORC1, VPS53, ZAN, ZNF141, ZNF407, ZNF419, ZNF469,
ZNF479

### Certain Selection Probes

Some embodiments include enriching for target nucleic acids in the amplified polynucleotides, such as products of mutagenesis PCR, and/or products of suppression PCR. In some such embodiments, the enriching comprises hybridizing a plurality of selection probes with the amplified polynucleotides, wherein the selection probes of the plurality of selection probes comprise different nucleotide sequences from one another. In some embodiments, the plurality of selection probes comprise at least 50, 100, 200, 500, 1000, 5000, or 10000 different selection probes.

In some embodiments, the selection probes are designed such that an average distance between two adjacent nucleotide sequences of the selection probes on a reference sequence of a genome, such as a human genome, is in a range from about 300 consecutive nucleotides to about 7,000 consecutive nucleotides; a range from about 500 consecutive nucleotides to about 5,000 consecutive nucleotides; a range from about 750 consecutive nucleotides to about 2,500 consecutive nucleotides; a range from about 750 consecutive nucleotides to about 1,500 consecutive nucleotides; a range from about 900 consecutive nucleotides to about 1,200 consecutive nucleotides. In some embodiments, an average distance between two adjacent nucleotide sequences of the selection probes on a reference sequence of a genome is about 750, 1000, 1500, or 2000 consecutive nucleotides.

In some embodiments, the selection probes are designed such that an average number of sites in a genome, such as a human genome, that each selection probe of the plurality of selection probes is capable of hybridizing to is no more than 100 different sites in the genome, no more than 50 different sites in the genome, no more than 40 different sites in the genome, no more than 30 different sites in the genome, no more than 20 different sites in the genome, no more than 10 different sites in the genome, or no more than 5 different sites in the genome, or any number between any of the foregoing number of different sites. In some embodiments, each selection probe of the plurality of selection probes is capable of hybridizing to no more than 100 different sites in the genome, no more than 50 different sites in a genome, no more than 40 different sites in a genome, no more than 30 different sites in a genome, no more than 20 different sites in a genome, or no more than 10 different sites in a genome, or any number between any of the foregoing number of different sites. In some embodiments, a selection probe capable of hybridizing to a site in the genome comprises at least 50, 60, 70, or 80 consecutive nucleotides complementary to at least 80%, 90%, 95%, 96%, 98%, or 100% of a nucleotide sequence at the site in the genome. In some embodiments, a selection probe capable of hybridizing to a site in the genome comprises at least 50 consecutive nucleotides complementary to at least 90% of a nucleotide sequence at the site in the genome.

In some embodiments, the plurality of selection probes lack sequences capable of hybridizing to a repetitive genomic DNA element. In some embodiments, the repetitive genomic DNA element is selected from a tandem repeat, an Alu repeat, a short interspersed nuclear element (SINE), a long interspersed nuclear element (LINE), an integrated viral sequence, a viral long terminal repeat (LTR), and a transposon.

In some embodiments, the selection probes target a plurality of selected genes, such as a panel including challenging medically relevant genes (CMRG), such as the genes listed in TABLE 1C. In some such embodiments, each selection probe of the plurality of selection probes comprises a nucleotide sequence capable of hybridizing to a gene selected from TABLE 1C; and/or a nucleotide sequence within, no more than 10 kbp 5′ or no more than 10 kbp 3′ of a gene selected from TABLE 1C. In some embodiments, each selection probe of the plurality of selection probes comprises a nucleotide sequence having at least 90%, 95%, or 100% sequence identity to any one of SEQ ID NOs:02-140601. In some embodiments, each selection probe of the plurality of selection probes comprises a nucleotide sequence having at least 90%, 95%, or 100% sequence identity to any one of SEQ ID NOs:02-43879. In some embodiments, each selection probe of the plurality of selection probes comprises a nucleotide sequence having at least 90%, 95%, or 100% sequence identity to any one of SEQ ID NOs:02-6936.

### Certain Compositions, Kits and Systems

Some embodiments of the methods and composition provided herein include kits and systems for preparing a nucleic acid library. Some embodiments include a kit comprising: a first bead-linked transposomes (BLT-1) reagent, wherein the BLT-1 transposomes comprises a first adaptor sequence; a mutagenesis reagent comprising a first primer, dPTPs, dNTPs, and a polymerase; a second bead-linked transposomes (BLT-2) reagent, wherein the BLT-2 transposomes comprise the first adaptor and a second adaptor; an amplification reagent comprising a first primer, a second primer, dNTPs, and a polymerase; wherein BLT-1 has a lower transposome density as compared to BLT-2; and wherein the first primer hybridizes to the first adaptor sequence and the second primer hybridizes to the second adaptor sequence. In some embodiments, BLT-2 has more than 10, 20, 50, 100, or 1000 times the transposome density as compared to BLT-1. In some embodiments, the first adaptor is B15 and the second adaptor is A14. Some embodiments include population of oligonucleotides, wherein the oligonucleotides comprise at least 10, 100, 1000, 10000 different nucleotide sequences selected from any one of SEQ ID NOs. 02-140601.

Some embodiments include system for preparing a nucleic acid library, comprising: (a) a first plurality of transposomes comprising transposon adaptors for tagmenting a plurality of nucleic acid fragments, wherein the first plurality of transposomes is immobilized on a first plurality of beads at a first density; (b) reagents for amplifying the plurality of polynucleotides to obtain amplified polynucleotides, wherein the amplifying comprising a mutagenesis PCR and/or a suppression PCR, wherein: (i) the first reagent for performing mutagenesis PCR comprise a low bias DNA polymerase and/or a nucleotide analogue; optionally, wherein the nucleotide analogue comprises dPTP, and/or 8-oxo-dGTP; and/or the low bias DNA polymerase is a Thermococcal polymerase, or a functional derivative thereof, optionally, wherein the Thermococcal polymerase is derived from a Thermococcus strain selected from the group consisting of T. kodakarensis, T. siculi, T. celer and T. sp KS-1, and (ii) the first reagents for performing suppression PCR comprise amplification primers having the same nucleotide sequence capable of hybridizing to the transposon adaptors; (c) a plurality of selection probes for enriching for target polynucleotides in the amplified polynucleotides; and (d) a second plurality of transposomes comprising library adaptors for adding library adaptors to each end of the amplified polynucleotides, wherein the second plurality of transposomes is immobilized on a second plurality of beads at a second density, wherein the first density is less than the second density.

Some embodiments include a system for preparing a nucleic acid library, comprising: (a) a first plurality of transposomes for tagmenting a plurality of nucleic acid fragments to obtain a plurality of polynucleotides; (b) first reagents for amplifying the plurality of polynucleotides to obtain amplified polynucleotides; and (c) second reagents for adding library adaptors to each end of the amplified polynucleotides. In some embodiments, the first plurality of transposomes comprises transposon adaptors, wherein the first plurality of transposomes is immobilized on a solid support. In some embodiments, the solid support comprises a first plurality of beads.

In some embodiments, the first plurality of the transposomes is immobilized on the first plurality of beads at a density such that on contacting the first plurality of transposomes with the plurality of nucleic acid fragments the plurality of polynucleotides has an average length of the plurality of polynucleotides is greater than about 1 kbp, 2 kbp, 5 kbp, 10 kbp, 15 kbp, 20 kbp, or 40 kbp. In some embodiments, the average length of the plurality of polynucleotides is in a range from about 1 kbp to about 40 kbp, 1 kbp to about 30 kbp, 1 kbp to about 20 kbp, 5 kbp to about 20 kbp, 5 kbp to about 15 kbp, or 7 kbp to about 12 kbp.

In some embodiments, the number of transposomes immobilized on the bead is no more than about 100 transposomes, 50 transposomes, 40 transposomes, 30 transposomes, 20 transposomes, or 10 transposomes. In some embodiments, the number of transposomes immobilized on the bead is no more than about 30 transposomes.

In some embodiments, the first plurality of the transposomes immobilized on the bead comprise a total activity such that on contacting the first plurality of transposomes with the plurality of nucleic acid fragments the plurality of polynucleotides has an average length greater than about 1 kbp, 2 kbp, 5 kbp, 10 kbp, 15 kbp, 20 kbp, or 40 kbp. In some embodiments, the average length of the plurality of polynucleotides is in a range from about 1 kbp to about 40 kbp, 1 kbp to about 30 kbp, 1 kbp to about 20 kbp, 5 kbp to about 20 kbp, 5 kbp to about 15 kbp, or 7 kbp to about 12 kbp.

In some embodiments, the first plurality of the transposomes immobilized on the bead comprise an activity in a range from about 0.05 AU/μl to about 0.25 0.05 AU/μl. In some embodiments, the first plurality of the transposomes immobilized on the bead comprise an activity of about 0.075 AU/μl.

In some embodiments, the transposon adapters comprise the same sequence. In some embodiments, the transposon adapters comprise the nucleotide sequence: SEQ ID NO:01 (GTCTCGTGGOCTCGG)

In some embodiments, the transposomes of the plurality of transposomes are the same. In some embodiments, the transposomes of the plurality of transposomes are B15 transposomes.

In some embodiments, the first reagents comprise reagents for performing mutagenesis PCR comprising a low bias DNA polymerase and/or a nucleotide analogue. In some embodiments, the nucleotide analogue comprises dPTP, and/or 8-oxo-dGTP. In some embodiments, the low bias DNA polymerase is a Thermococcal polymerase, or a functional derivative thereof. In some embodiments, the Thermococcal polymerase is derived from a Thermococcal strain selected from the group consisting of T. kodakarensis, T. siculi, T. celer and T. sp KS-1.

In some embodiments, the first reagents comprise reagents for performing suppression PCR comprising amplification primers having the same nucleotide sequence. In some embodiments, the amplification primers are capable of hybridizing to the transposon adaptors.

In some embodiments, the second reagents comprise a second plurality of transposomes comprising the library adaptors. In some embodiments, the second plurality of transposomes has an activity such that on contacting the second plurality of transposomes with the amplified polynucleotides a library of nucleic acids is obtained and comprises the library adaptors and having an average length less than about 1 kb, 900 bp, 800, bp, 700 bp, 600 bp, 500 bp, 400 bp, 300 bp, 200 bp, or 100 bp.

In some embodiments, the first plurality of the transposomes is immobilized on the beads at a density less than a density at which the second plurality of transposomes are immobilized on the second plurality of beads.

Some embodiments also include third reagents for enriching for target polynucleotides in the amplified polynucleotides, comprising a plurality of selection probes. In some embodiments, the plurality of selection probes is attached to a third plurality of beads.

In some embodiments, an average distance between two adjacent nucleotide sequences of the selection probes on a reference sequence of a genome is in a range from about 300 consecutive nucleotides to about 7,000 consecutive nucleotides. In some embodiments, the range is from about 500 consecutive nucleotides to about 5,000 consecutive nucleotides. In some embodiments, the range is from about 750 consecutive nucleotides to about 2,500 consecutive nucleotides. In some embodiments, the range is from about 750 consecutive nucleotides to about 1,500 consecutive nucleotides. In some embodiments, the range is from about 900 consecutive nucleotides to about 1,200 consecutive nucleotides. In some embodiments, an average distance between two adjacent nucleotide sequences of the selection probes on a reference sequence of a genome is about 750, 1000, 1500, or 2000 consecutive nucleotides.

In some embodiments, an average number of sites in a genome that each selection probe of the plurality of selection probes is capable of hybridizing to is no more than 50 different sites in the genome, to no more than 40 different sites in the genome, to no more than 30 different sites in the genome, to no more than 20 different sites in the genome. In some embodiments, each selection probe of the plurality of selection probes is capable of hybridizing to no more than 50 different sites in a genome, to no more than 40 different sites in a genome, to no more than 30 different sites in a genome, to no more than 20 different sites in a genome. In some embodiments, a selection probe capable of hybridizing to a site in the genome comprises at least 50, 60, 70, or 80 consecutive nucleotides complementary to at least 90% of a nucleotide sequence at the site in the genome.

In some embodiments, the plurality of selection probes lack sequences capable of hybridizing to a repetitive genomic DNA element; In some embodiments, the repetitive genomic DNA element is selected from a tandem repeat, an Alu repeat, a short interspersed nuclear element (SINE), a long interspersed nuclear element (LINE), an integrated viral sequence, a viral long terminal repeat (LTR), and a transposon.

In some embodiments, the plurality of selection probes comprise at least 50, 100, 200, 500, 1000, 5000 different selection probes.

In some embodiments, each selection probe of the plurality of selection probes comprises a nucleotide sequence capable of hybridizing to a gene selected from TABLE 1C; and/or a nucleotide sequence within, no more than 10 kbp 5′ or no more than 10 kbp 3′ of a gene selected from TABLE 1C. In some embodiments, each selection probe of the plurality of selection probes comprises a nucleotide sequence having at least 90%, 95%, or 100% sequence identity to any one of SEQ ID NOs:02-140600. In some embodiments, each selection probe of the plurality of selection probes comprises a nucleotide sequence having at least 90%, 95%, or 100% sequence identity to any one of SEQ ID NOs:02-43879. In some embodiments, each selection probe of the plurality of selection probes comprises a nucleotide sequence having at least 90%, 95%, or 100% sequence identity to any one of SEQ ID NOs:02-22546.

In some embodiments, the plurality of nucleic acid fragments is mammalian. In some embodiments, the plurality of nucleic acid fragments is human. In some embodiments, the plurality of nucleic acid fragments comprises genomic DNA.

Some embodiments include a kit comprising a plurality of selection probes. In some embodiments the plurality of selection probes comprise at least 50, 100, 1000, 2000, 3000, 4000, 5000, 10000, 20000, 30000, or 40000 or any number between any one of the foregoing numbers of different nucleotide sequences.

In some embodiments, each selection probe of the plurality of selection probes comprises a nucleotide sequence capable of hybridizing to a gene selected from TABLE 1C; and/or a nucleotide sequence within, no more than 10 kbp 5′ or no more than 10 kbp 3′ of a gene selected from TABLE 1C. In some embodiments, each selection probe of the plurality of selection probes comprises a nucleotide sequence having at least 90%, 95%, or 100% sequence identity to any one of SEQ ID NOs:02-140600. In some embodiments, each selection probe of the plurality of selection probes comprises a nucleotide sequence having at least 90%, 95%, or 100% sequence identity to any one of SEQ ID NOs:02-43879. In some embodiments, each selection probe of the plurality of selection probes comprises a nucleotide sequence having at least 90%, 95%, or 100% sequence identity to any one of SEQ ID NOs:02-22546.

### Examples

### Example 1—Generation of Nucleic Acid Library

The following describes the preparation of human genome libraries for sequencing with (1) low-density bead-linked transposomes; (2) random mutagenesis; and (3) bottlenecking (suppression) amplification. The workflow included uniquely encoding long DNA templates with steps including highly uniform random mutagenesis and amplification. The long DNA templates were then fragmented and sequenced on a standard short-read (SR) platform. Reads from prepared libraries were used to accurately reconstruct and decode the original long template sequences using an unmutated reference data set which was generated in parallel.

Unmutated reference data: to reconstruct accurate long read sequences from mutated short reads, an additional unmutated reference data set was used. This was generated from the same genomic starting material as the sample to be mutated, using standard methods for short-read library preparation and sequencing. Paired end reads were generated at a minimum length of 2×150 nucleotides for the unmutated data set, with a recommended 60× genome coverage for isolated bacterial genomes and 40× for pure human cell cultures.

Input DNA requirements: the workflow was found to be compatible with genomic DNA samples of relatively poor quality, containing unwanted low molecular weight fragments. These low molecular weight fragments were actively excluded by certain steps in the workflow; and the presence of some higher molecular weight material (>20 kb) was included to generate long templates for sequencing. To quantify input DNA for library preparation, a fluorometric-based method such as the Qubit dsDNA HS Assay Kit (Thermo Scientific) was used. Concentrations of input DNA between 12.5 and 50 ng/l was used.

The following outlines a workflow which included: high molecular weight tagmentation; mutagenesis PCR; library normalization; bottlenecking (suppression) PCR; library preparation; fragment analysis of products; and sequencing.

### Reagents, Materials and Thermocycler Parameters

| | |
|---|---|
| BLT Long Range | BLT-LR |
| Long Range PCR Mix | LPM |
| Long Range Marking Mix | LMM |
| Long Range Primer Mix 1 | LRP1 |
| Long Range Primer Mix 2 | LRP2 |
| Library Reducing Beads | LRB |
| Binding Buffer | IBB |
| Wash Buffer 1 | IWB1 |
| Stop Tagment Buffer 2 | ST2 |
| Illumina Purification Bead | IPB |
| PW1 (water) | |
| Resuspension Buffer | RSB |
| Ethyl Alcohol, Pure | |
| Enrich BL Transposome, Lg-LT | eBLT-L |
| Enhanced PCR Mix | EPM |
| Tagmentation Buffer 1 | TB1 |
| Tagmentation Wash Buffer | TWB |
| Nextera DNA UD Index Plate | |

Tagmentation parameters

| Step | Temperature | Time |
|---|---|---|
| 1 | 55° C. | 5 minutes |
| 2 | 10° C. | hold |

Mutagenesis PCR parameters

| Step | Temperature | Time | |
|---|---|---|---|
| 1 | 68° C. | 3 | minutes |

| | |
|---|---|
| 2 | PCR cycles (12 cycles) |

| | | |
|---|---|---|
| 98° C. | 10 | seconds |
| 68° C. | 10 | minutes |

| | | |
|---|---|---|
| 3 | 4° C. | hold |

Bottlenecking PCR parameters

| | | |
|---|---|---|
| Step | Temperature | Time |

| | |
|---|---|
| 1 | PCR cycles (14 or 16 cycles) |

| | | |
|---|---|---|
| 98° C. | 10 | seconds |
| 68° C. | 10 | minutes |

| | | |
|---|---|---|
| 3 | 4° C. | hold |

Index PCR parameters

| Step | Temperature | Time | |
|---|---|---|---|
| 1 | 68° C. | 3 | minutes |
| 2 | 98° C. | 3 | minutes |

| | |
|---|---|
| 3 | PCR cycles (6 cycles) |

| | | | |
|---|---|---|---|
| | 98° C. | 45 | seconds |
| | 62° C. | 30 | seconds |
| | 68° C. | 2 | minute |
| 4 | 68° C. | 1 | minutes |

| | | |
|---|---|---|
| 5 | 4° C. | hold |

### High Molecular Weight Tagmentation

This step used low density bead-linked transposomes (BLT-LR) to generate long DNA fragments tagged with adapter sequences.

| | |
|---|---|
| 1 | Label a Biorad PCR plate. |
| 2 | Label a 1.7 ml tube and add the following components in order: BLT-LR; and |
| | TB1. |
| 3 | Vortex to mix thoroughly, then briefly pulse centrifuge. |
| 4 | Add 20 μl of mastermix to wells. |
| 5 | Add 30 μl input DNA (50 ng total). |
| 6 | Use a pipette to gently mix then briefly pulse centrifuge. |
| 7 | Place tubes in a thermocycler with a heated lid set to 80° C. and run the Tagment. |
| 8 | Remove from the thermocycler and add 10 μl of ST2. |
| 9 | Use a pipette set at half of the total rxn volume to gently mix. |
| 10 | Incubate at room temperature for 2 min. |
| 11 | Place the tubes on a magnetic stand or plate for 3 min or until the solution is |
| | clear. Remove and discard the clear supernatant. Note: If beads become |
| | disturbed during aspiration, redisperse solution into the tube. Maintain tube on |
| | the magnet to let the beads settle. |
| 12 | Remove tubes from the magnet and add 100 μl TWB. Gently pipette mix until |
| | beads are fully resuspended. |
| 13 | Place the tubes on a magnetic stand or plate and incubate for at least 2 min, or |
| | until clear. While samples are incubating, proceed to the next step. Note: Keep |
| | pellets in TWB to help prevent any chance of over-drying the beads. |

### Mutagenesis PCR

In this step, long templates were uniquely encoded via random incorporation of the mutagenic nucleotide analogue dPTP during PCR.

| | |
|---|---|
| 14 | Label a 1.7 ml tube and combine the following to prepare mutagenesis master |
| | mix: LMM; LPM; LRP1; and RSB. |
| 15 | Use a pipette to mix thoroughly, then briefly pulse centrifuge. |
| 16 | Remove TWB from each well. |
| 17 | Add 50 μl of master mix to beads. |
| 18 | Gently pipette mix to resuspend beads |
| 19 | Place in preprogrammed thermal cycler and run the Mutagenesis PCR program. |
| 20 | Remove from thermocycler. |
| 21 | Vortex IPB to resuspend the beads. |
| 22 | Add 30 μl of IPB beads to each well. |
| 23 | Mix the total reaction volume by gently pipetting at least 10 times and incubate |
| | for 2 minutes at room temperature. |
| 24 | Place on the magnetic stand and wait until the liquid is clear (~5 minutes). |
| 25 | Remove and discard all supernatant. |
| 26 | Wash beads as follows: a. keep on the magnetic stand and add 180 μl fresh 80% |
| | EtOH to each well; b. wait 30 seconds; and c. remove and discard all |
| | supernatant |
| 27 | Briefly spin for 10 seconds and return plate to magnet. |
| 28 | With a 20 μl pipette, remove all residual EtOH. |
| 29 | Dry on magnet for 2 minutes. |
| 30 | Remove from the magnetic stand and add 25 μl RSB to each well. |
| 31 | Pipette mix and incubate at room temperature for 2 minutes. |
| 32 | Place on the magnetic stand and wait until the liquid is clear (~2 minutes). |
| 33 | Transfer 10 μl supernatant to a new plate and leave remaining supernatant with |
| | SPRI beads |

### Library Normalization

| | |
|---|---|
| 34 | In a new 2 ml tube, combine the following to prepare normalization master mix: |
| | Binding Buffer (IBB); and LRB |
| 35 | Vortex normalization master mix until homogenous immediately prior to use. |
| 36 | Pipette 10 μL of prepared mastermix into each well containing 10 μL of DNA. |
| 37 | Seal plate with microseal and put on bioshake for 30 minutes at 1800 rpm. |
| 38 | Remove plate from bioshake and place on plate magnet for 1 minute. Removing |
| | and replacing plate on magnet may help to condense beads. |
| 39 | Gently remove supernatant, taking care not to disturb beads. |
| 40 | Remove plate from magnet. |
| 41 | Immediately add 150 μL of IWB1 to each well. |
| 42 | Using a p200 pipette set to 140 μL, pipette mix each well at least 10 times. |
| 43 | Place on plate magnet for 1 minute. |
| 44 | Gently remove supernatant, taking care not to disturb beads. |
| 45 | Repeat steps 41-44 for a total of two washes. Remove plate from magnet. |
| 46 | Immediately add 150 μL of 80% EtOH to each well. |
| 47 | Using a p200 pipette set to 140 μL, pipette mix each well at least 10 times. |
| 48 | Place on plate magnet for 1 minute. |
| 49 | Gently remove supernatant, taking care not to disturb beads. |
| 50 | Briefly spin down plate and use a p20 to remove excess liquid. |
| 51 | Allow to dry for 2 minutes. |
| 52 | Add 80 μL of RSB to each sample well. |
| 53 | Using a pipette set to 50 μL, pipette mix each sample at least 10 times. |
| 54 | Allow to incubate for 10 minutes. |
| 55 | Place on plate magnet for 1 minute. |
| 56 | Transfer 70 μL of supernatant to new well. |
| 57 | If performing 165 pg input, go to step 62 for next |
| | If performing 55 pg input, dilute 20 ul of eluted material in 40 μl of RSB in |
| | adjacent well. Mix well, then use 2 μl as stated in step 62. |
| | [Optional] Measure DNA on Qubit using 10 μL of DNA diluted in 190 μL of |
| | Qubit buffer. |

### Bottlenecking (Suppression) PCR

In this step a defined quantity of the purified mutagenesis product was amplified to create many copies of each unique template. The amount of starting material in the bottlenecking PCR determined the number of long templates available for sequencing, and was controlled through careful dilution of the mutagenesis sample. The following protocol was used to generate between about 10× to about 30× long-read coverage of the human genome (see below). For arbitrary samples, a simple calculator or look up table was, provided to guide users on sample dilution and indicate the number of enrichment cycles required for a particular genome size or sample type.

| | |
|---|---|
| 58 | Combine the following volumes to prepare enrichment master mix: LPM; and |
| | LRP2. |
| 59 | Use a pipette to mix thoroughly, then briefly pulse centrifuge. |
| 60 | Add 48 μl of master mix to a new Bio-Rad plate. Place tubes on ice. |
| 61 | Add 2 μl of DNA to each well. |
| 62 | Gently pipette mix the 50 μl reaction and briefly pulse centrifuge. |
| 63 | Place in preprogrammed thermal cycler and run the Bottlenecking PCR program. |
| 64 | Note: Perform 16 enrichment cycles for 10x genome coverage or 14 cycles for |
| | 30x. |
| 65 | Remove from thermocycler. |
| 66 | Vortex IPB to resuspend the beads. |
| 67 | Add 30 ul of IPB beads to each well. |
| 68 | Mix the total reaction volume by gently pipetting at least 10 times and incubate |
| | for 2 minutes at room temperature. |
| 69 | Place on the magnetic stand and wait until the liquid is clear (~5 minutes). |
| 70 | Remove and discard all supernatant. |
| 71 | Wash beads as follows: a. keep on the magnetic stand and add 180 μl fresh 80% |
| | EtOH to each well; b. wait 30 seconds; and c. remove and discard all supernatant. |
| 72 | Briefly spin for 10 seconds and return plate to magnet. |
| 73 | With a 20 μl pipette, remove all residual EtOH. |
| 74 | Dry on magnet for 2 minutes. |
| 75 | Remove from the magnetic stand and add 25 μl RSB to each well. |
| 76 | Pipette mix and Incubate at room temperature for 2 minutes. |
| 77 | Place on the magnetic stand and wait until the liquid is clear (~2 minutes). |
| 78 | Transfer 24 μl supernatant to a new plate. |
| 79 | Keep on ice for same day use or store at −20° C. (safe stopping point). |
| 80 | Determine the concentration of each purified bottlenecking PCR product with the |
| | Qubit dsDNA HS Assay Kit. Use 1-2 μl of sample DNA per measurement. |

### Library Preparation

In this step the long, mutated DNA templates were fragmented and adapters were attached to create a library of short, overlapping fragments that are ready for sequencing. 50 ng of each purified enrichment product can be used as input DNA for internal library preparation. A small amount of bottlenecking PCR product was reserved for subsequent analysis of template size.

| | |
|---|---|
| 81 | Bring TB1 and eBLT-L to room temperature and vortex to mix. |
| 82 | Make a dilution using RSB and 50 ng worth of purified bottlenecking product |
| | into each well with a final volume of 30 μl. |
| 83 | Vortex eBLT-L vigorously for 10 seconds, then visually check the beads for |
| | complete resuspension. Repeat as necessary. |
| 84 | Prepare a tagmentation master mix: eBLT-L; TB1. |
| 85 | Vortex the tagmentation master mix thoroughly to make sure the eBLT-L |
| | beads are evenly resuspended in the buffer. |
| 86 | Using fresh tips, transfer 20 μl of tagmentation master mix to each tube |
| | containing 30 μl of DNA. |
| 87 | Pipette mix the 50 μl reaction mix to resuspend. |
| 88 | Place in preprogrammed thermal cycler and run the Tagment program. |
| 89 | Remove from thermocycler and add 10 μl of ST2 to each tagmentation |
| | reaction. |
| 90 | Use a pipette set at half of the total rxn volume to gently mix. |
| 91 | Incubate at RT for 5 minutes. |
| 92 | Place the tubes on a magnetic stand or plate for 3 min or until the solution is |
| | clear. Remove and discard the clear supernatant. Note: If beads become |
| | disturbed during aspiration, redisperse solution into the tube. Maintain tube on |
| | the magnet to let the beads settle. |
| 93 | Remove tubes from the magnet and add 100 μl TWB. Gently pipette mix until |
| | beads are fully resuspended. |
| 94 | Place the tubes on a magnetic stand or plate for 3 min or until the solution is |
| | clear. Remove and discard the clear supernatant. |
| 95 | Remove tubes from the magnet and add 100 μl TWB. Gently pipette mix until |
| | beads are fully resuspended (total of 2 washes). |
| 96 | Place the tubes on a magnetic stand or plate and incubate for at least 3 min, or |
| | until clear. While samples are incubating, proceed to the next step. Note: Keep |
| | pellets in TWB to help prevent any chance of over drying the beads. |
| 97 | Prepare a index PCR master mix: EPM; RSB. |
| 98 | Use a pipette to mix thoroughly, then briefly pulse centrifuge. |
| 99 | Carefully remove the second TWB wash from the samples while on the |
| | magnet. Use a pipette with P10 or P20 tips to remove excess liquid from the |
| | tube. Any remaining foam on the tube walls does not adversely affect the |
| | library. |
| 100 | Remove tubes from the magnet and proceed immediately to the next step to |
| | prevent excessive drying of the beads. |
| 101 | Add 46 μl of the PCR master mix to each sample. Gently pipette mix to ensure |
| | the beads are thoroughly resuspended. |
| 102 | To each sample, add 4 μl Nextera DNA UD Index. Use a unique combination |
| | of indexes for each sample. |
| 103 | Using a pipette set to 40 μl, pipette mix a minimum of 10 times to mix the |
| | entire reaction volume. |
| 104 | Place in preprogrammed thermal cycler and run the Index PCR program. |
| 105 | Clean up PCR reaction using IPB beads as follows: |
| 106 | Place plate on the magnetic stand and wait until the liquid is clear (~5 |
| | minutes) |
| 107 | Transfer 45 μL of supernatant to a new plate. |
| 108 | Add 55 μL RSB to bring volume to 100 uL. |
| 109 | Perform a double-sided bead purification as follows: |
| 110 | Add 30 μL of IPB beads to the sample. |
| 111 | Mix the total reaction volume by gently pipetting at least 10 times and |
| | incubate for 2 minutes at room temperature. |
| 112 | Place on the magnetic stand and wait until the liquid is clear (~5 minutes). |
| 113 | Transfer 127 μl of the clear supernatant in new wells. Discard the remaining |
| | beads. |
| 114 | Add 20 μl of IPB beads to the sample. |
| 115 | Mix the total reaction volume by gently pipetting at least 10 times and |
| | incubate for 2 minutes at room temperature. |
| 116 | Place on the magnetic stand and wait until the liquid is clear (~5 minutes). |
| 117 | Remove and discard all supernatant. |
| 118 | Wash beads as follows: a. keep on the magnetic stand and add 180 μl fresh |
| | 80% EtOH to each well; b. wait 30 seconds; and c. remove and discard all |
| | supernatant. |
| 119 | Briefly spin for 10 seconds and return plate to magnet. |
| 120 | With a 20 μl pipette, remove all residual EtOH. |
| 121 | Dry on magnet for 2 minutes. |
| 122 | Remove from the magnetic stand and add 30 μl RSB to each well. |
| 123 | Pipette mix and incubate at room temperature for 2 minutes. |
| 124 | Place on the magnetic stand and wait until the liquid is clear (~2 minutes). |
| 125 | Transfer 28 μl to a new plate. |
| 126 | Determine the concentration of each purified enrichment product with the |
| | Qubit dsDNA HS Assay Kit. Use 1-2 μl of sample DNA per measurement. |
| 127 | Keep on ice for same day use or store at −20° c. (SAFE STOPPING POINT). |

Libraries were quantified, normalized and sequenced using standard workflows for libraries. Example additional considerations for quality control, sample index selection and sequencing are provided below.

### Fragment Analysis of Products

| | |
|---|---|
| 128 | Run purified library prep product on appropriate |
| | instrument for fragment analysis. |
| 129 | Record the average fragment length for the purified |
| | library prep product. |

DNA fragment length: assessing the fragment length profile of the purified bottlenecking PCR product was performed to evaluate the size distribution of long templates as well as to evaluate the final short-read library. To assess the fragment size of the purified bottlenecking PCR product, the following products from Agilent Technologies® were used: Bioanalyzer 2100, TapeStation 4200, and Fragment Analyzer 5300; or equivalent technologies from other providers.

Purified Bottlenecking Product

| | | | Expected average |
|---|---|---|---|
| | | Recommended | fragment length |
| Instrumentation | Kit | Region (bp) | (bp) |
| Bioanalyzer | High Sensitivity | 3,000-10,000 | 7,000-8,000 |
| 2100 | DNA | | |
| Fragment | DNF-464 HS Large | 3,000-49,000 | 7,000-8,000 |
| Analyzer 5300 | Fragment | | |

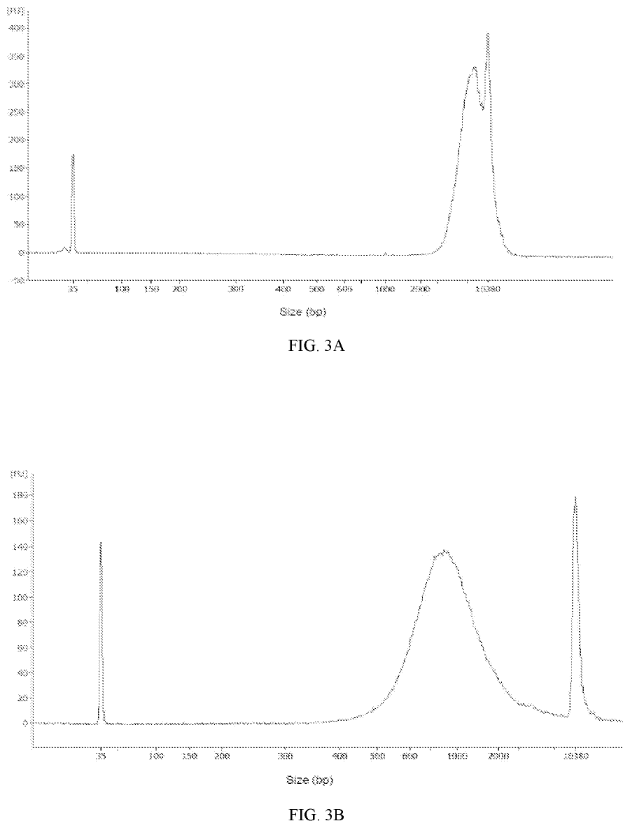

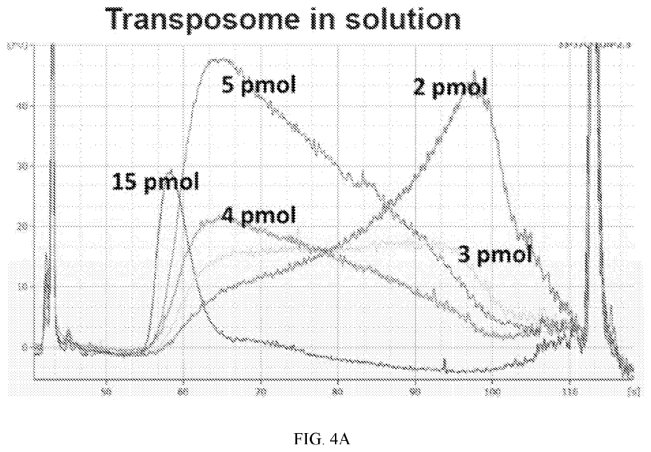

The peak template length after bottlenecking PCR was expected to be around 7,000-8,000 bp, with virtually no products below ˜3,000 bp. FIG. 3A illustrates purified bottlenecking PCR product run on an Agilent® Bioanalyzer using a High Sensitivity DNA Kit.

Final Library

| | | Region | Expected average |
|---|---|---|---|
| Instrumentation | Kit | (bp) | fragment length (bp) |
| TapeStation | High Sensitivity | 150-1,500 | 800-900 |
| 4200 | D5000 | | |
| Bioanalyzer | High Sensitivity DNA | 150-1,500 | 800-900 |
| 2100 | | | |

The peak template length after bottlenecking PCR was expected to be around 800-900 bp. FIG. 3B illustrates purified final library prep product run on an Agilent® Bioanalyzer using a High Sensitivity DNA Kit.

### Sequencing

The final library or library pool was sequenced on a NGS instrument, generating 2×150 nt paired end reads. The aim was to produce at least 400 Gbp of sequence data for mutated samples targeting 10× long-read coverage of the human genome, or at least 1200 Gbp for 30× coverage. This was in addition to the unmutated reference data that was also required for long read reconstruction.

### Example 2—Effects of Immobilizing Transposomes on Beads at Low Densities

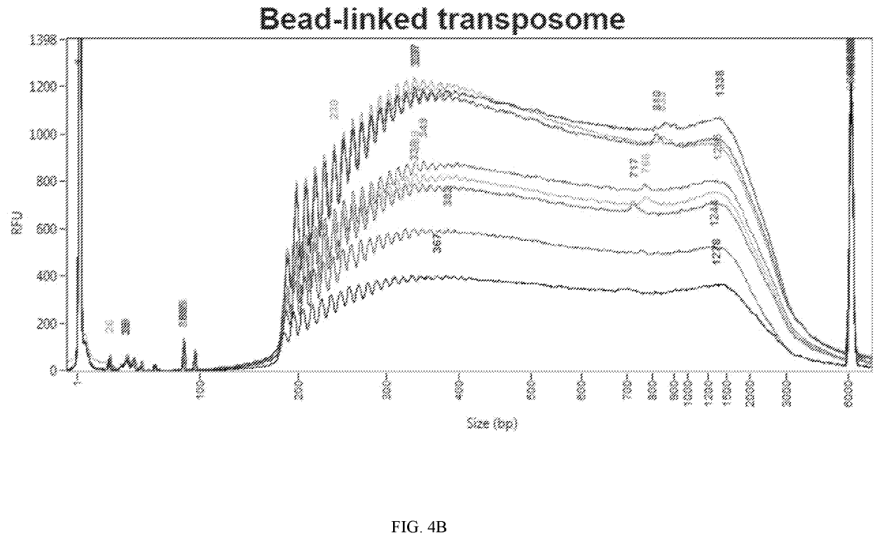

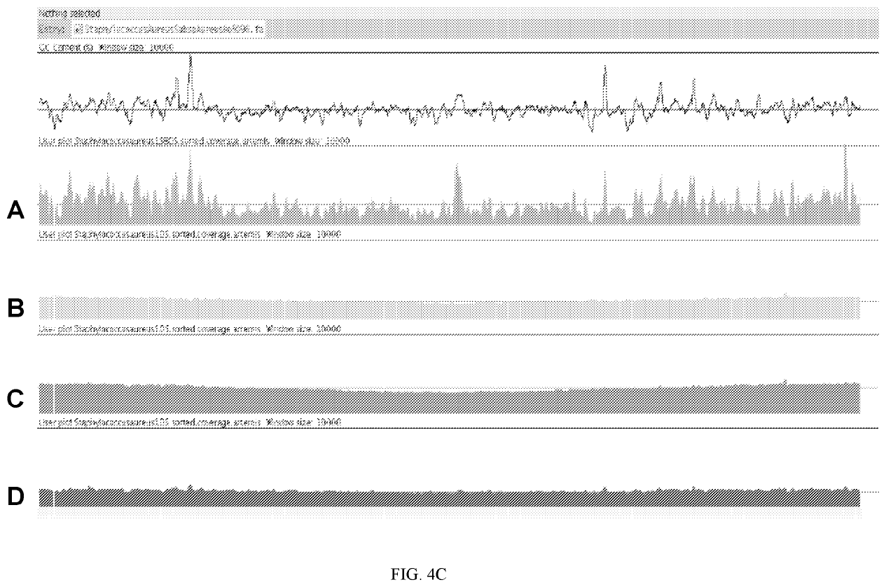

This example illustrates improved long read coverage by changing initial tagmentation from soluble transposomes to low density bead-linked transposomes (BLT-LR); and changing from an A14/B15 mixture of BLT-LRs to B15 BLT-LR only. Nucleic acid libraries were generated and sequenced with a protocol substantially similar to that of Example 1. Different amounts of input DNA were tested. A protocol using bead-linked transposomes was compared with a protocol using transposomes in solution. As shown in FIGS. 4A-4C, a switch from low concentration soluble transposomes to low-density BLT (BLT-LR) provided increased robustness to changes in transposome:input DNA ratio; and a more uniform coverage with BLT vs. soluble.

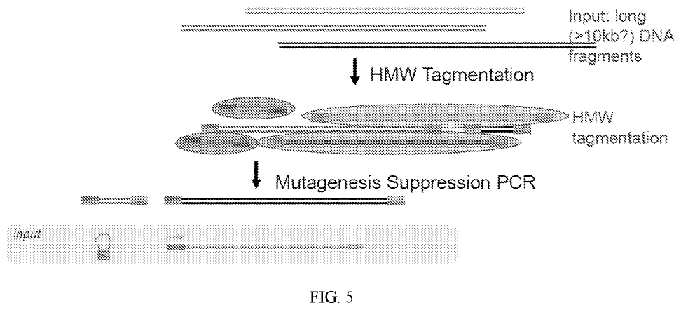

FIG. 5 outlines steps of high molecular weight tagmentation followed by mutagenesis and suppression PCR to enrich for longer fragments.

Protocols were compared that included (i) soluble transposome (TSM) (0.4 AU/ul); (2) BLT-LR made with A14/B15 (0.1 AU/ul build); or (3) BLT-LR made with B15 only (0.075 AU/ul build). Quality control and sequencing metrics were compared for each protocol.

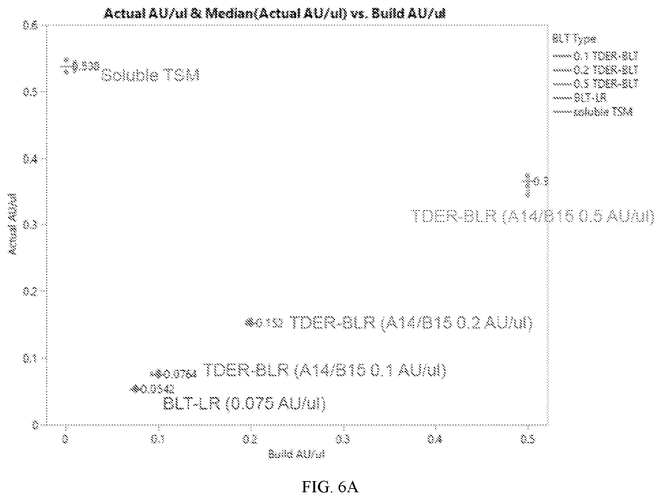

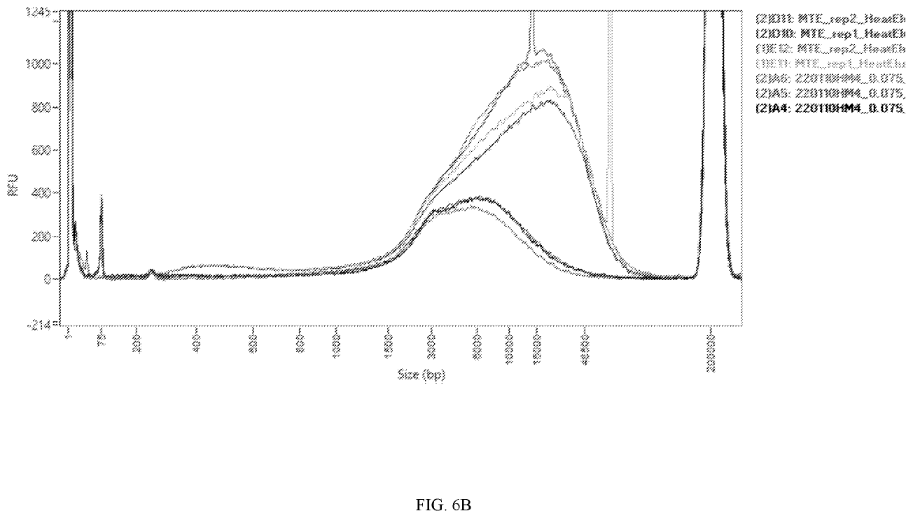

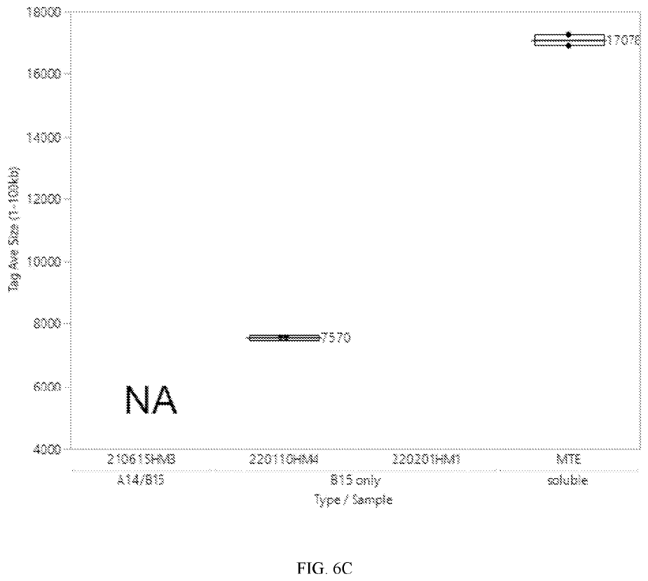

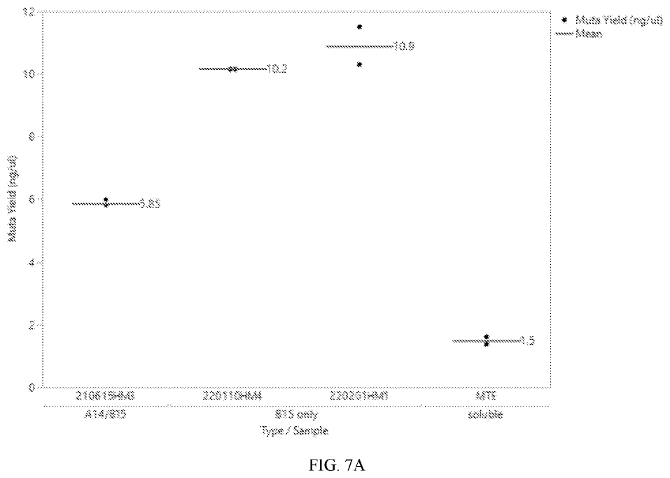

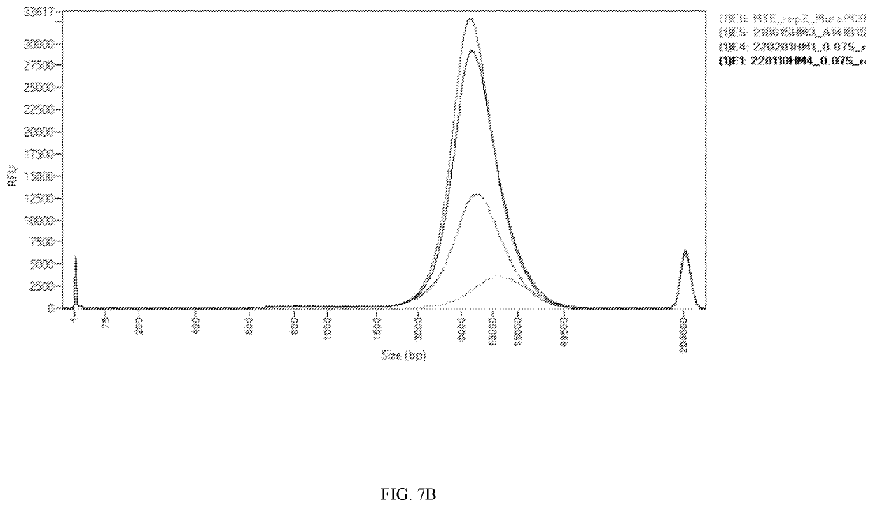

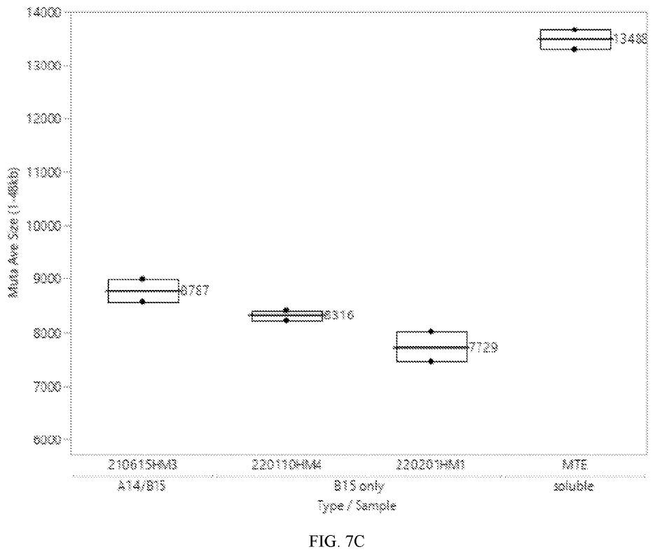

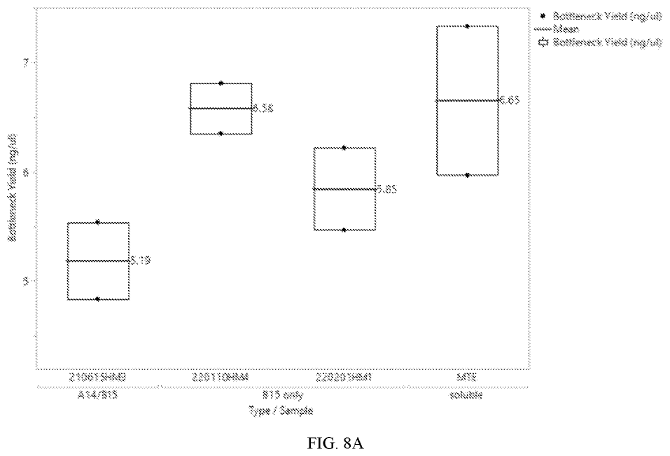

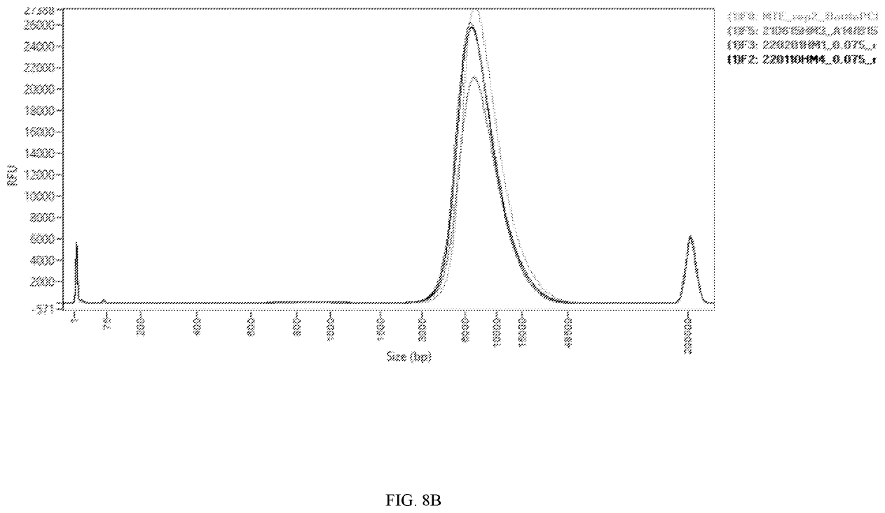

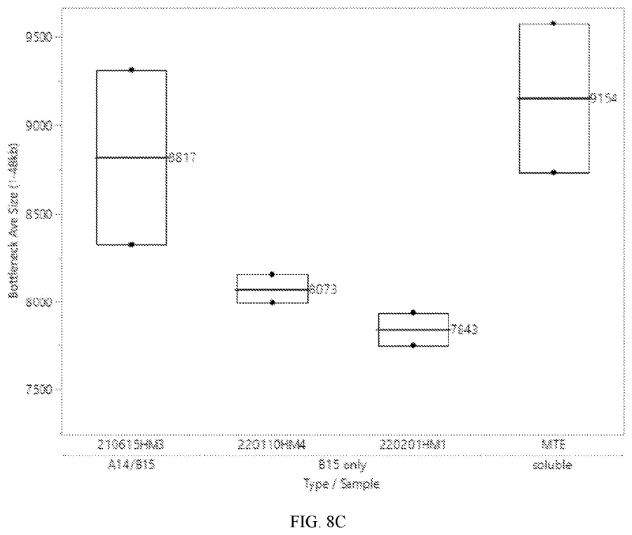

As shown in FIGS. 6A-6C, soluble TSM had a greater activity than BLT-LR and soluble TSM created longer fragments than BLT-LRs. A14/B15 could not be melted off of the beads due to 5′ attachment of TDE1. In mutagenesis PCR, A14/B15 BLT provided a lower yield than B15 only (FIGS. 7A-7C). The yield with soluble (NITE) yield was also lower and may have been accounted for because only 50% tag product was taken into PCR; 100% used for BLTs. Fragment sizes of BLT-LR were smaller than with soluble TSM. Average sizes of products were 1-48 kb (FIG. 7B). There were larger fragment lengths with soluble TSM (FIGS. 7B and 7C). In bottleneck (suppression) PCR, yields and fragment sizes were more similar, while BLT-LRs still produced smallest products of ˜8 kb in length (FIG. 8A). Average sizes were 1-48 kb (FIGS. 8B and 8C).

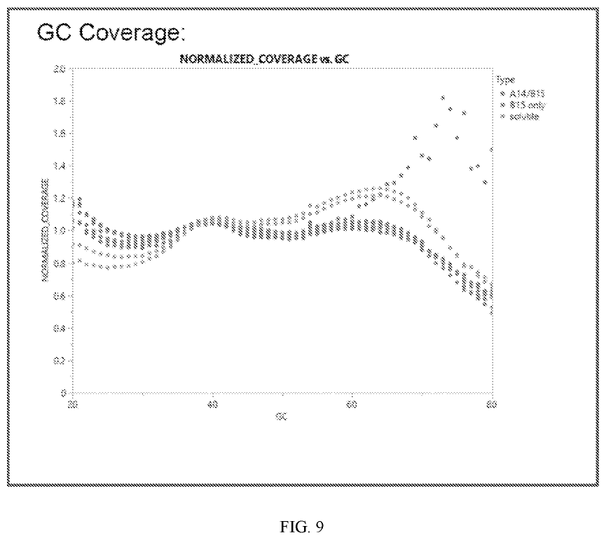

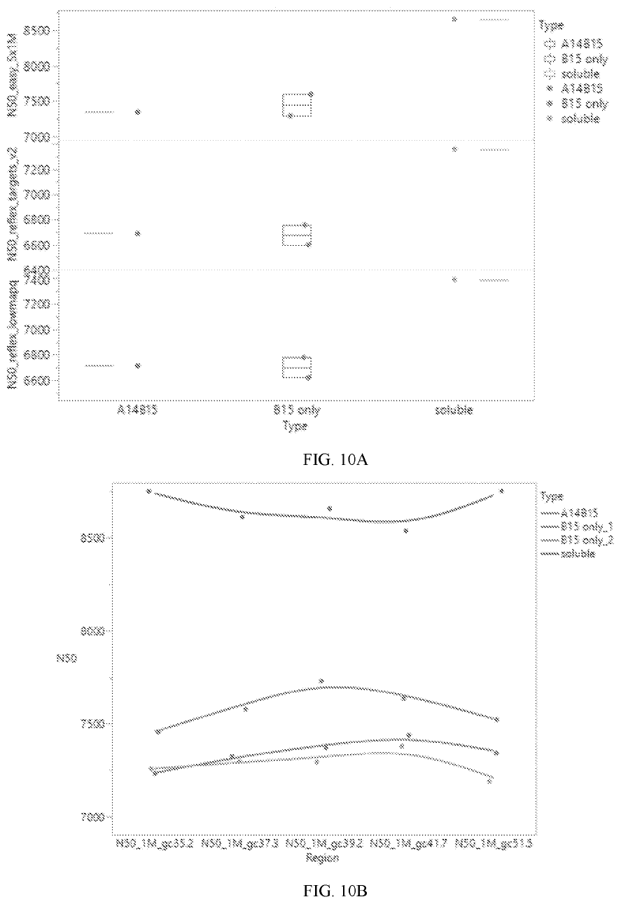

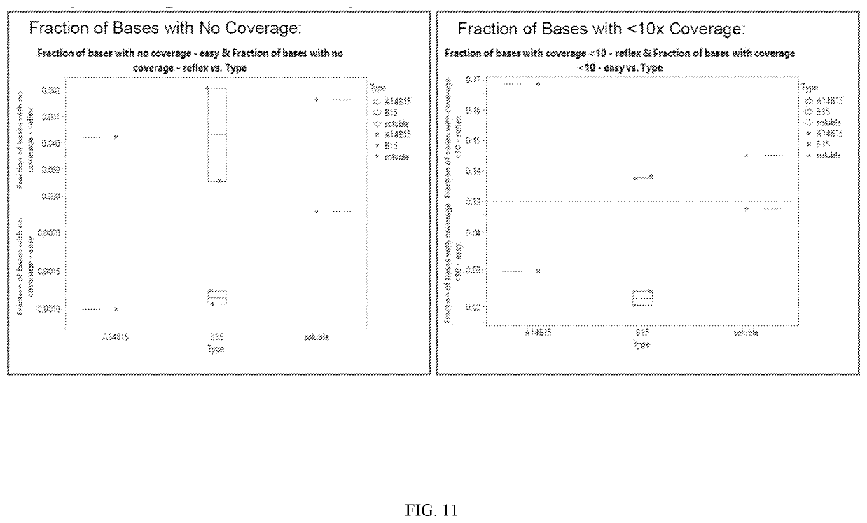

In a sequencing metrics comparison, GC bias was comparable between soluble and BLT-LR with A14/B15 BLT slightly worse (FIG. 9) A higher N50 was achieved with soluble TSM, slightly higher N50 with B15-only compared to A14/B15, slightly higher N50 (350) with eBLT-L compared to BLT (FIGS. 10A and 10B). Sequencing redundancy, SLR depth, and error rate achieved using with A14/B15 BLT or B1 only BLT were measured. Fraction of bases with no coverage achieved using with A14/B15 BLT or B15 only BLT is depicted in FIG. 11.

BLT-LRs had better coverage than soluble transposomes (fraction of bases with <0/10× coverage). BLT-LRs created shorter fragments, which generated lower N50s in workflow. N50s were still above 5 kb mark so this decrease in performance was acceptable when paired with better coverage metrics and a more robust tagmentation reaction. B15-only TSM BLTs gave a better yield and lower redundancy than A14/B15 BLTs. The change to B15 BLT-LRs created a more efficient mutagenesis suppression PCR.

### Example 3—Effects of BLT Activity on Fragment Size

BLT-LR activity was investigated for high molecular weight tagmentation. BLT-LR activity should provide: tagment large fragments to provide for mutagenesis PCR; maximize fragment size, ideally >8 kb; yield >4 ng post-high molecular weight (HMW) tagmentation; reproducibility, ease in QC tested, and good sequence quality. A goal was to maximize fragment size while maintaining good yield and downstream sequencing metrics.

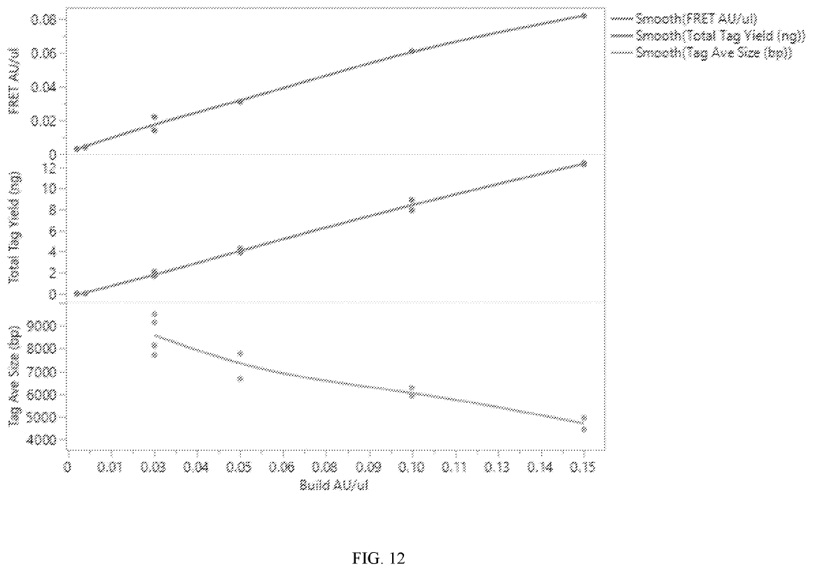

BLT-LRs having different levels of activity were compared. As transposome activity (AU/ul) decreased, yield decreased and average fragment size increased (FIG. 12).

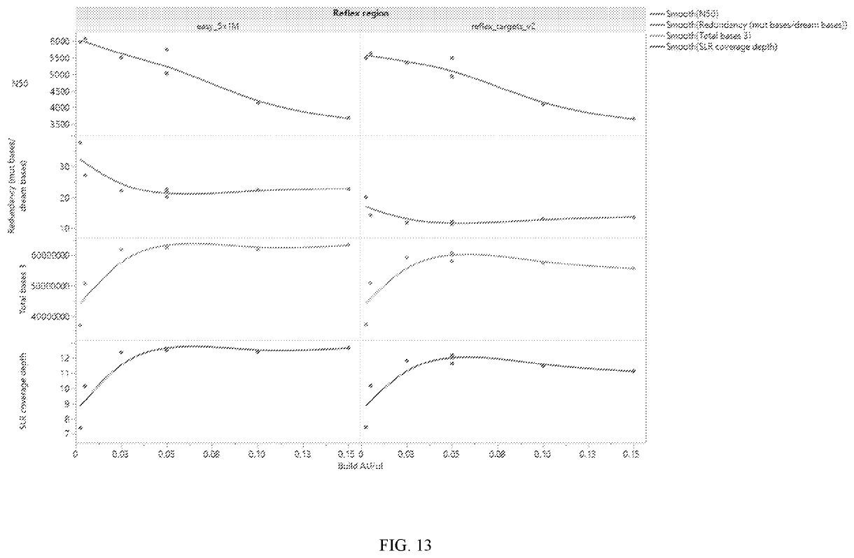

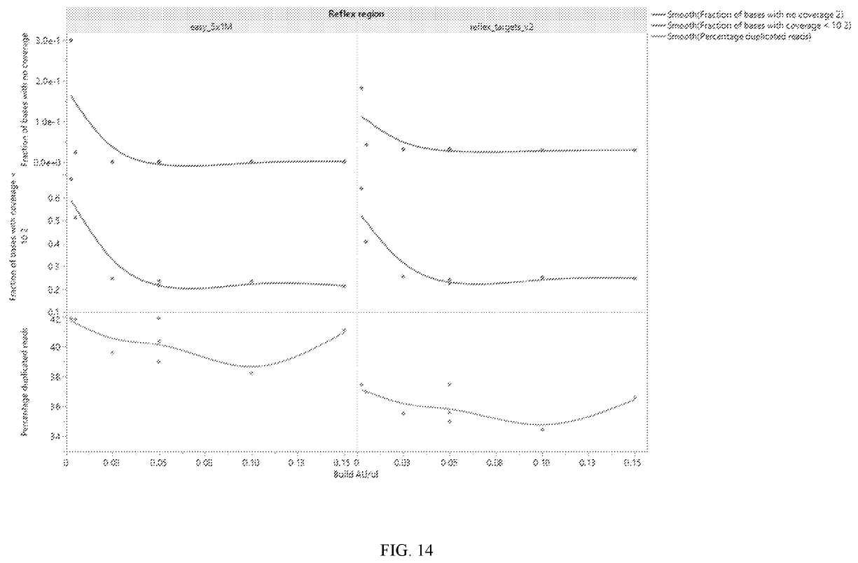

BLT-LRs were compared with builds/activities of 0.0025, 0.005, 0.025, 0.05, 0.1, 0.15 AU/ul. N50s were compared. “N50” was the length of the shortest contig for which longer and equal length contigs cover at least 50% of the assembly. Lower build activity maximized N50s but sequencing metrics started to drop at 0.025 AU/ul (FIGS. 13 and 14). There was no apparent cliff-edge on high activity side, but N50s continued to decline, and at 0.075 AU/ul, were well above cliff edge while maximizing N50.

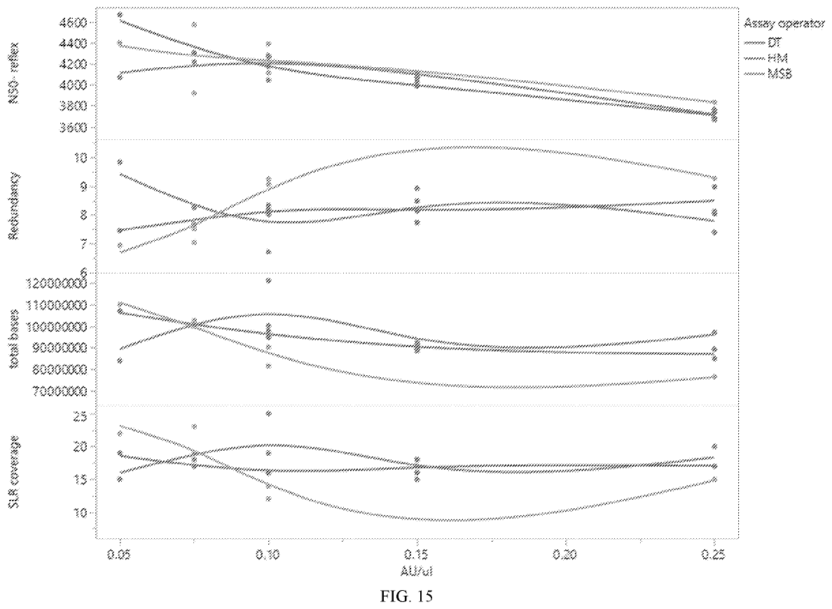

Results were compared from studies with three different operators testing activities from 0.05 AU/ul to 0.25 AU/ul. Consistent performance between operators was found for BLT-L R activities from 0.05 AU)/ul to 0.25 AU/ul (FIG. 15), A BLT-LR activity of 0.075 AU/ul was chosen for BLT-LR which balanced fragment size and yield. It was found that a fluctuation of +/−100% in activity would still provide good sequencing metrics.

### Example 4—Effects of Quantity of Input DNA

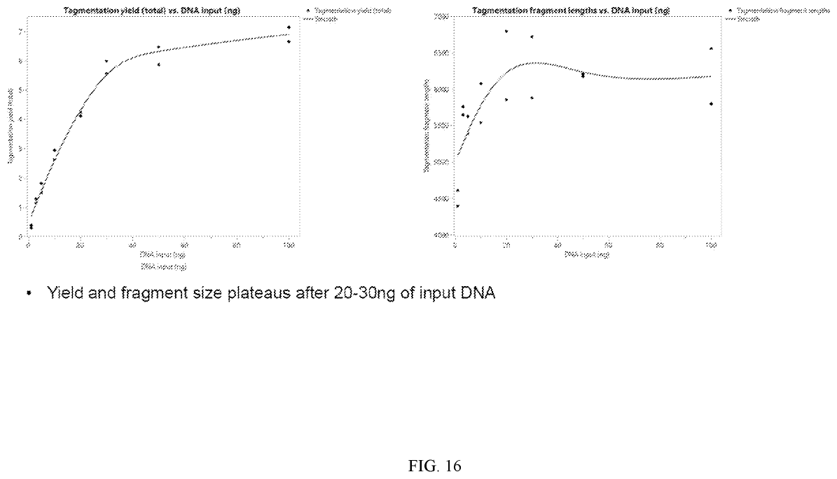

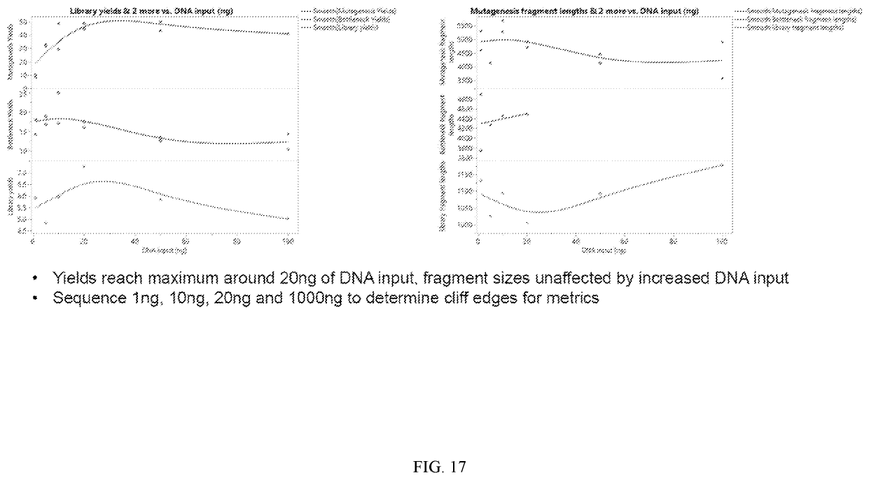

Changing the amount of input DNA used in the initial HMW tagmentation reaction could impact any of the following: amount of DNA tagmented (test BLT-LR saturation); fragment sizes after initial HMW tagmentation (and downstream); biases in what is tagmented/amplified; sequencing metrics including percent duplicates, redundancy, N50, CC bias. Effects of input DNA quantity were tested for a protocol substantially similar to the workflow of Example 1 for amounts: 1 ng, 3, ng, 5 ng, 10 ng, 20 ng, 30 ng, 50 ng, 100 ng, 300 ng, and 1000 ng. Yield and fragment size plateaued after 20-30 ng of input DNA (FIG. 16). Yields reached maximum around 20 ng of DNA input, fragment sizes were unaffected by increased DNA input (FIG. 17).

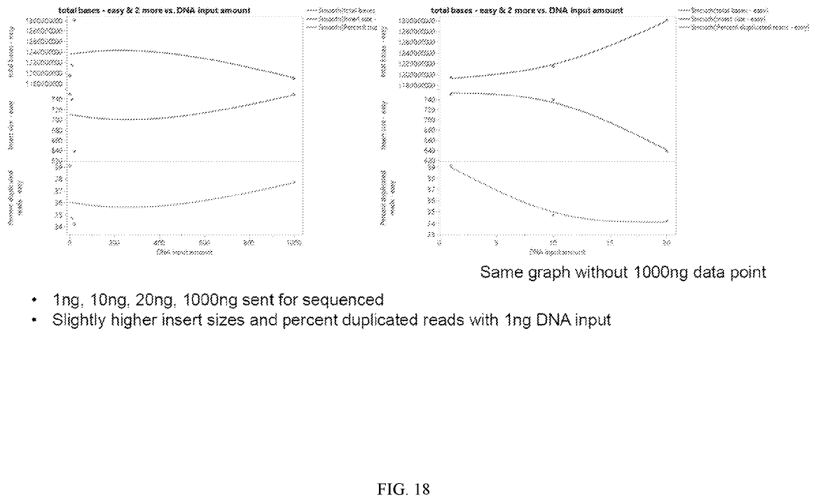

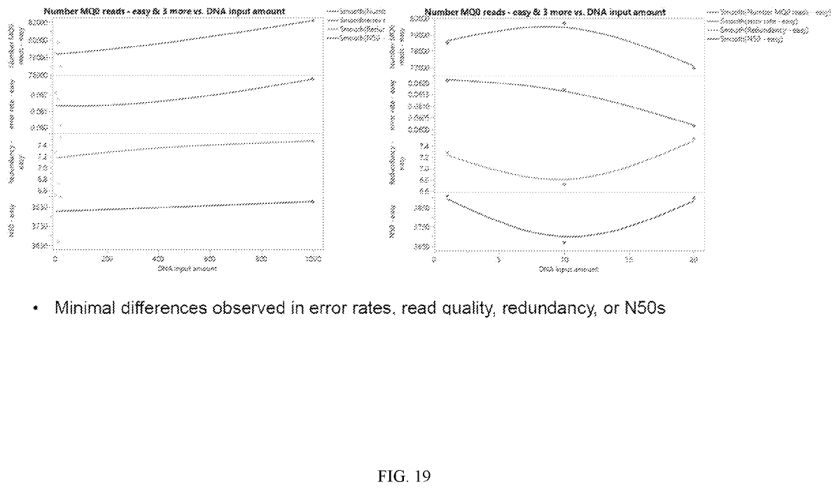

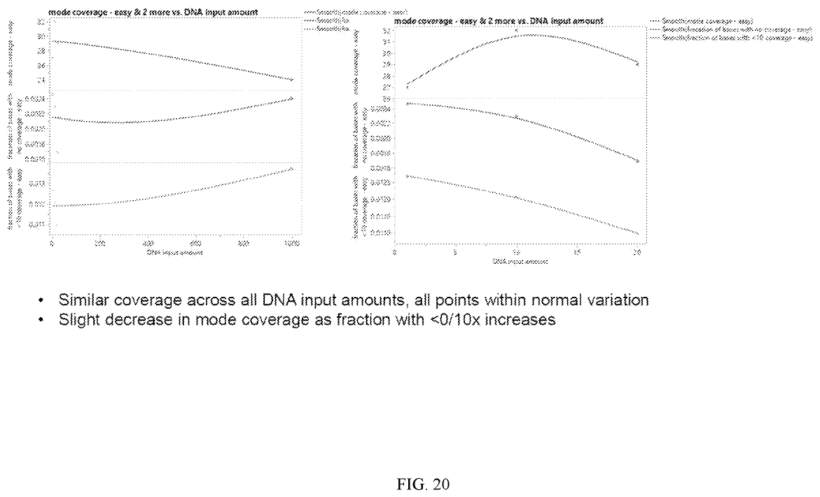

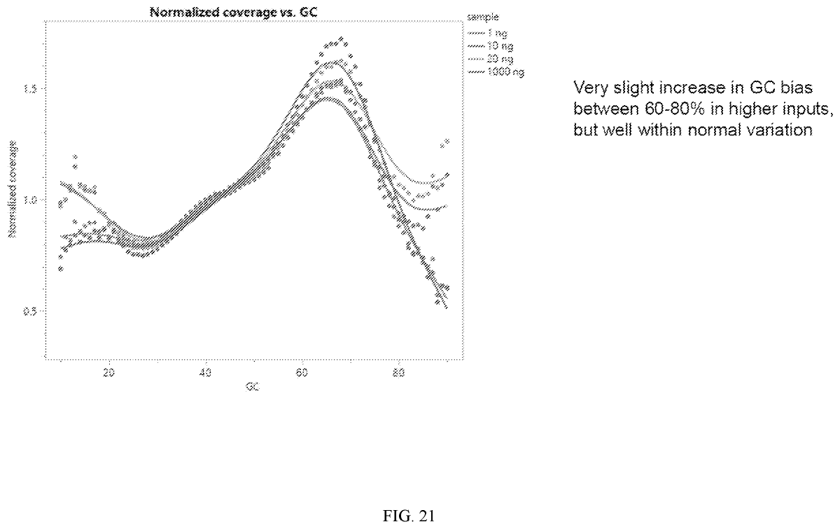

Sequencing was performed for input DNAs of 1 ng, 10 ng, 20 ng and 1000 ng to determine cliff edges for metrics. A workflow with 1 ng input DNA provided slightly higher insert sizes and percent duplicated reads compared to workflows performed with higher amounts of input DNA (FIG. 18). Minimal differences were observed in error rates, read quality, redundancy, or N50s (FIG. 19). Similar coverage across all DNA input amounts were observed with all points within normal variation with a slight decrease in mode coverage as fraction with <0/0× increased (FIG. 20). A very slight increase in GC bias between 60-80% was observed in higher inputs, but well within normal variation (FIG. 21).

In sum, similar sequencing metrics were observed for input DNA amounts of 1 ng to 1000 ng. Inputs as low as 1 ng were found to work within the workflow. BLT-LR saturated at about 30 ng input; input above 30 ng did not bias coverage. DNA input amounts less than 10 ng resulted in low yields in the workflow. There was a slight increase in percent duplicated reads for 1 ng DNA input.

### Example 5-Effects of Quality of Input DNA

Effects of input DNA quality was tested for a protocol substantially similar to the workflow of Example 1. Preliminary data indicated that vortexing and up to 3 freeze/thaw cycles for input DNA were tolerated; and FFPE DNA was not suitable, or was too degraded, to be used as input DNA. Input DNA was prepared by shearing for 1, 3, 10, 30 and 60 seconds, and compared to control BLT-LR tagmented DNA and HMW DNA.

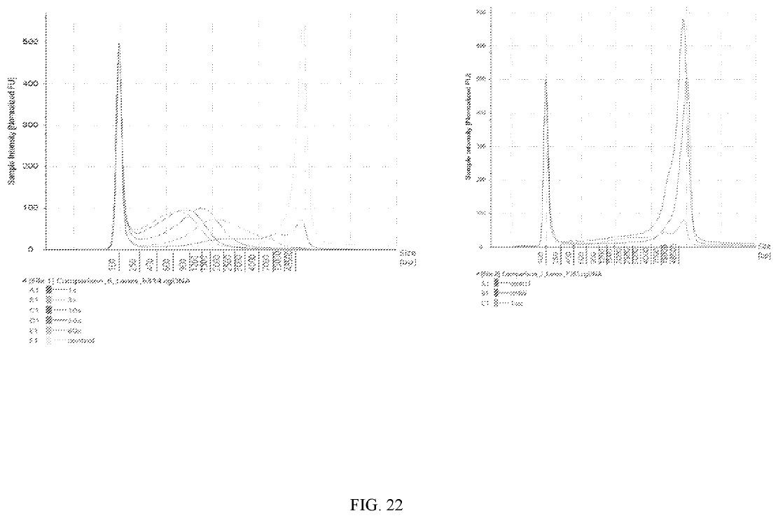

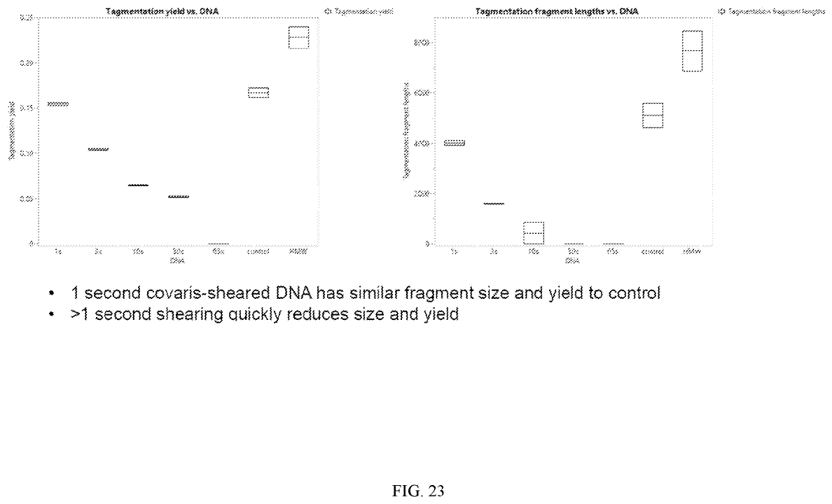

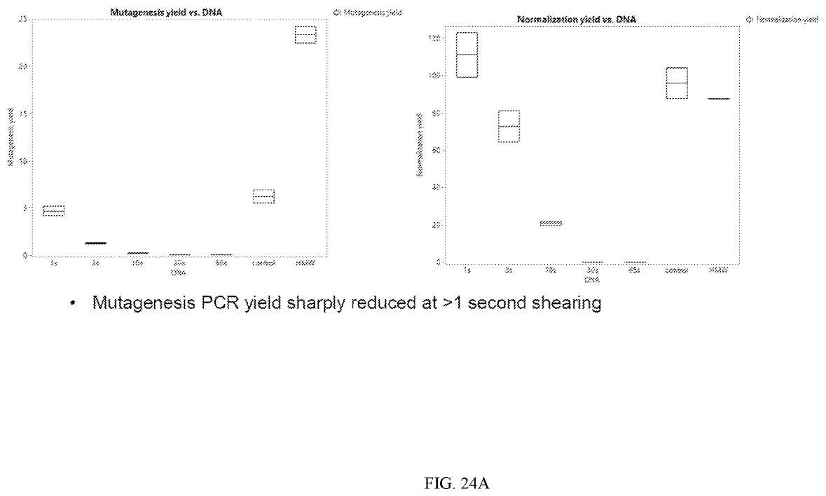

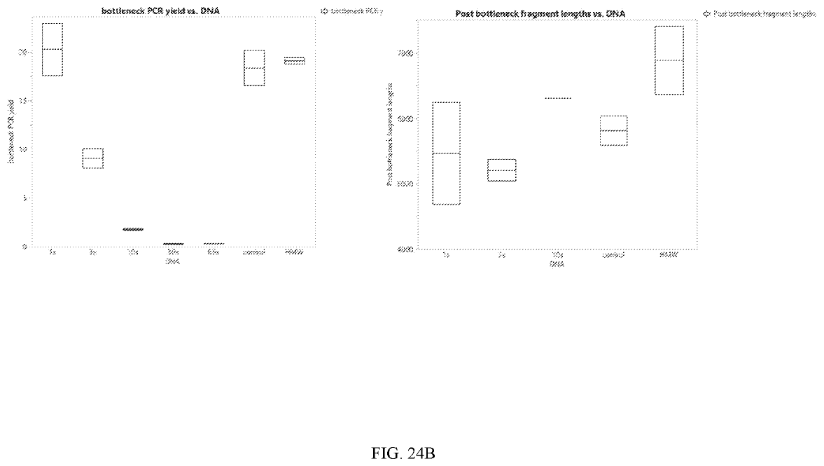

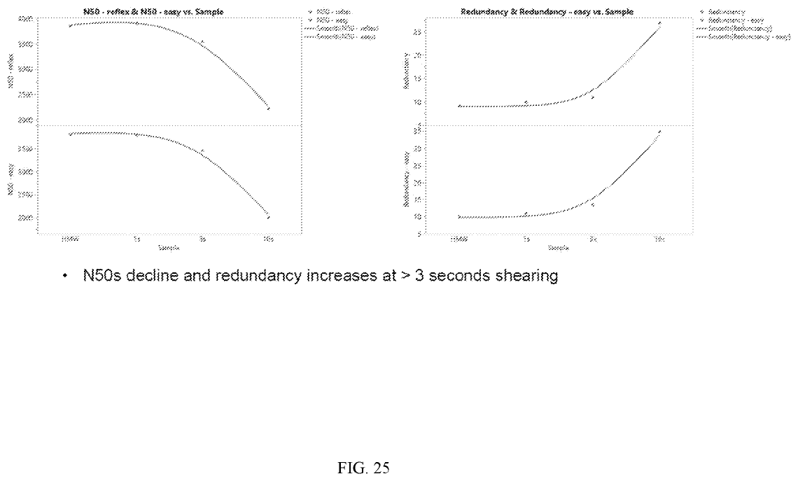

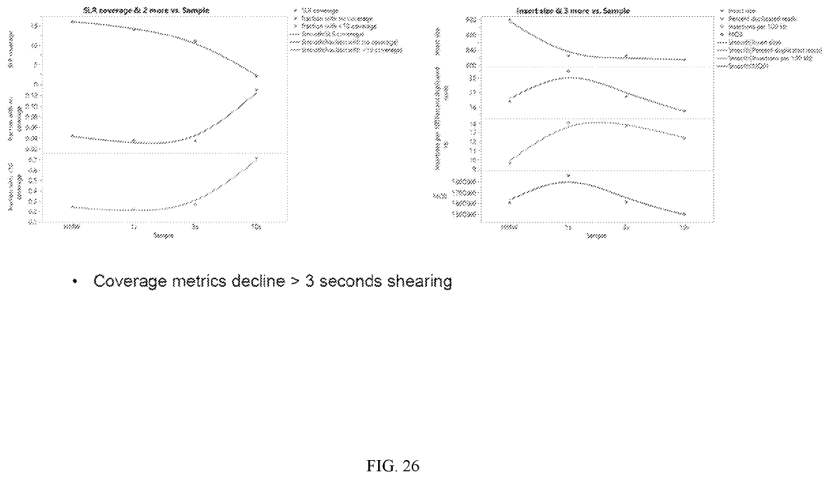

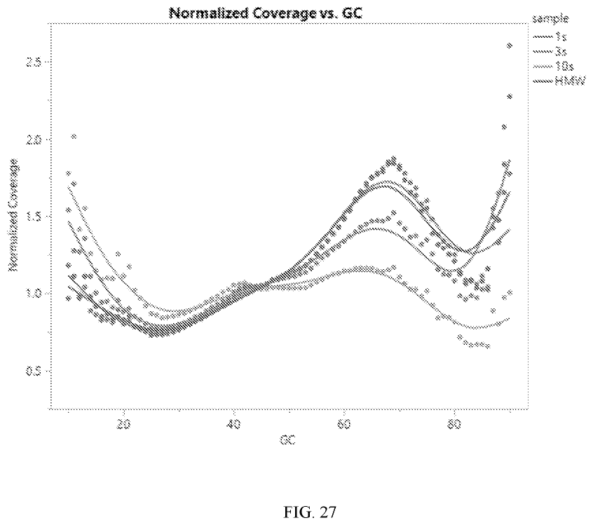

Input DNA was sheared for 1, 3, 10, 30 and 60 seconds. There was a noticeable change in size distribution profile after even 1 second, while Control BLT-LR and HMW DNA gave similar size profiles (FIG. 22). In the tagmentation step of the workflow, 1 second sheared DNA had similar fragment size and yield to control, and >1 second shearing quickly reduced size and yield (FIG. 23). In the mutagenesis step of the workflow, mutagenesis PCR yield sharply reduced at >1 second shearing (FIG. 24A). In the bottlenecking PCR step of the workflow, yield also sharply reduced at >1 second shearing (FIG. 24B). For sequencing metrics, N50s declined and redundancy increased at >3 seconds shearing (FIG. 25). Coverage metrics declined >3 seconds shearing (FIG. 26). GC bias correlated with post-tagmentation sizes (FIG. 27).

In sum, HMW DNA gave better yields and larger fragment lengths coming out of initial Tagmentation but did not result in final higher N50. Highly sheared DNA (30s or longer) did not amplify well in either PCR step which resulted in not enough DNA to continue with library prep. N50s and coverage/redundancy metrics worsened with DNA sheared >3 seconds. GC bias was impacted by fragment sizes. PCR steps were not suitable for highly degraded DNA, but tolerated mild shearing (1-3 see) reasonably well.

BLT-LRs were investigated to create large fragment sizes suitable for the workflow outlined in Example 1. B15-only transposomes improved mutagenesis PCR small fragment suppression and overall yield The workflow gave improved coverage of low MapQ regions of the genome. The workflow was robust to changes in DNA input amount. The workflow tolerated mildly sheared DNA, DNA that has been through freeze/thaw, and DNA that had been vortexed.

### Example 6—Enriching for Long Fragments

A workflow was performed, as described in Example 1, with an added enrichment step. The workflow included: high molecular weight tagmentation; mutagenesis PCR; library normalization; bottlenecking (suppression) PCR; library preparation; fragment analysis of products; and sequencing. FIG. 32 depicts an example workflow which includes enrichment for long fragments. The additional enrichment step for long fragment enrichment of certain fragments was performed on the products of the bottlenecking (suppression) PCR, and prior to the library preparation step. The fragments that were products of the suppression PCR, are referred to as ‘long fragments’ to differentiate them from fragments that are the products of the library preparation step and referred to in Example 7 as ‘short fragments’.

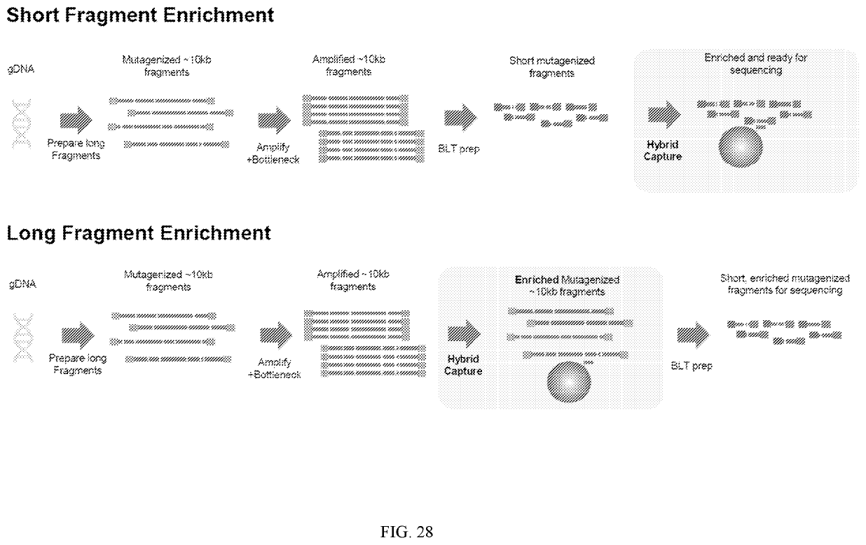

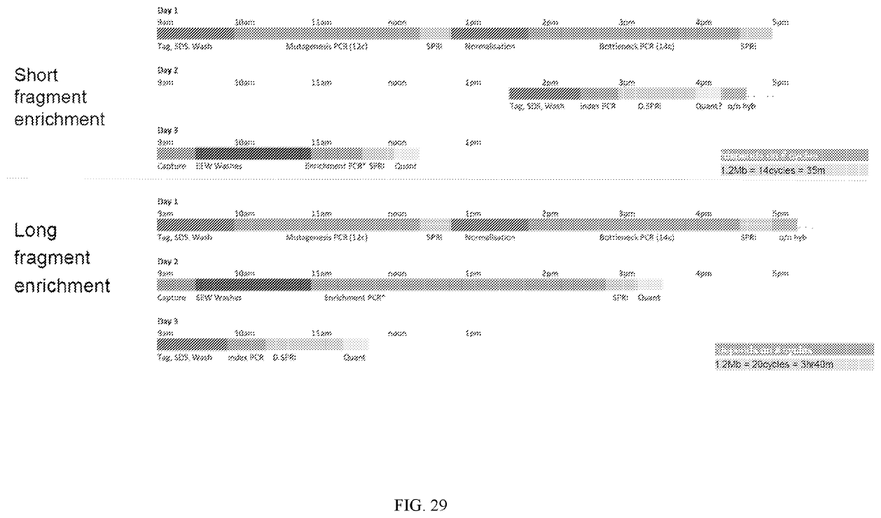

An example overview and timeline for long fragment enrichment in the workflow is depicted in FIG. 28 and FIG. 29, respectively. The enrichment step included hybridizing the products with selection probes, capturing the products hybridized to the selection probes with bead-linked capture probes, and amplifying the captured products. An example protocol is described below in the following. However, it should be realized that other protocols using similar enrichment for long fragments are contemplated.

### Hybridization

Bring DNA libraries (HYB Plate), EHB2, and probes to RT.
Vortex and briefly spin down EHB2 and probes before use.
NOTE: Vortex EHB2 thoroughly and allow enough time to bring
up to RT.
Ensure that crystals are fully dissolved. Heat tube in hands
if needed.
Preheat NHB2 to 50° C. for 5 m and vortex for 30 sec, ensure
solution is clear and free from precipitate.
NOTE: Vortex NHB2 thoroughly and allow enough time to thaw to RT
and get up to 50° C. prior to use.
Ensure that crystals are fully dissolved. Use while warm to avoid
precipitates from reforming.
Prepare Hyb Master Mix and add 17.5 uL to HYB plate wells. (or add
components individually in order: Library, NHB2, Probes, EHB2)
Pipette mix. Seal HYB plate with Microseal ‘B’.
Centrifuge HYB plate briefly at 280 × g for 30 sec.
Place plate on a thermal cycler and run the LCE HYB program
overnight. (holds at 58° C.)

| | | |
|---|---|---|
| Program name: LCE HYB | Rnx volume: 25μ | Lid: 100° C. |

| Step | Temp | Duration | | Cycles |
|---|---|---|---|---|
| Heat denature | 98° C. | 5 | min | 1 |
| Ramp down | 96° C. | −2° | C./min | 19 |
| | 58° C. | | | |
| Hybridize | 58° C. | 30 | min | 1 |

### Capture

Bring SMB3, EEW, EE1, HP3, ET2, and RSB to RT.
Vortex and briefly spin down ET2, EE1 and HP3 before use.
Vortex EEW and SMB3 thoroughly.
Leave the thermal cycler running at 58 C.
Remove the HYB plate from the thermal cycler.
Centrifuge HYB plate at 280 × g for 30 sec.
Vortex SMB3 thoroughly for 1 min before use.
Add 75 μL of SMB3 to each sample.
Pipette mix 20x
Seal HYB wash plate with Microseal ‘B’
Incubate plate on thermal cycler running at 58° C. for 15 min.
Centrifuge HYB wash plate briefly at 280 × g for 30 sec.
Place on a magnetic stand for 2 min.
While on the magnetic stand, use a pipettor set to 100 μL to
carefully remove and discard the supernatant.

### Post-Hybridization Wash

Vortex EEW thoroughly for 1 min before use.
Vortex and briefly spin down EE1 and HP3 before use.

| | |
|---|---|
| PERFORM | Remove the HYB plate from the magnetic stand |
| 2X | and add 100 μL of EEW to each sample. |
| | Pipette mix to fully resuspend the beads. |
| | Seal HYB wash plate with Microseal ‘B’ |
| | Incubate in a thermal cycler at 58° C. for 10 min. |
| | Place HYB plate on a magnetic stand for 2 min. |
| | While on the magnetic stand, use a pipettor to |
| | carefully remove and discard the supernatant. |

Use a P20 pipettor to remove any residual supernatant from the final
pellet.
Remove HYB plate from magnet.
Add 100 μL EEW to each well.
Pipette mix to fully resuspend the beads.
Transfer 100 μl of resuspended bead solution to a new PCR plate.
Seal HYB wash plate with Microseal ‘B’
Place on the thermal cycler at 58° C. for 5 min.
Turn off thermal cycler at 58° C.
Remove from thermalcycler and place on magnetic stand for 2 min
or until the solution is clear.
Prepare EE1 + HP3 Elution Mix.
Set pipette to 100 μL and discard all supernatant from each well.
Centrifuge at 280 × g for 30 sec then return to magnet for 10 sec.
Use a P20 pipettor to remove any residual supernatant.
Immediately proceed to Elution to prevent excessive drying of beads.

### Elution

Vortex and briefly spin down ET2 before use.
Vortex EE1 + HP3 Elution Mix to mix well.
Remove the HYB plate from the magnetic stand and add 23 μL
of EE1 + HP3 Elution Mix.
Pipette mix to fully resuspend the beads.
Incubate at RT for 2 min.
Centrifuge at 280 × g for 30 sec.
Place on a magnetic stand for 2 min.
Add 4 μL of ET2 to a new 96-well PCR plate to wells corresponding
to sample layout.
Label the plate, “ELU plate”.
Carefully transfer 21 μL of eluate from the HYB wash plate to
corresponding wells of ET2 in the ELU plate.
Set pipette to 20 μL and gently pipette mix each well 10 times.
Centrifuge at 280 × g for 30 sec.

Enriched library PCR

Bring MM reagents to RT.
Prepare RED Master Mix. Mix and spin.
Briefly spin down ELU plate to collect condensation if
needed.
Pipette mix.
Seal ELU plate with Microseal ‘B’
Briefly spin down plate to collect droplets.
Place plate on a thermal cycler and run the SYD_EN PCR
program.
(# cycles depends on Probe Panel used)
(same PCR conditions as Bottleneck PCR)

Enrichment PCR

| | | |
|---|---|---|
| Step | Temperature | Time |

| | |
|---|---|
| 1 | PCR cycles (depend on panel) |

| | | |
|---|---|---|
| 98° C. | 10 | s |
| 68° C. | 10 | min |

| | | |
|---|---|---|
| 3 | 4° C. | Hold |

### PCR Clean Up

Bring IPB and RSB to RT for at least 30 min.
Prepare fresh 80% EtOH wash solution as follows. Vortex to mix well.
Remove ELU plate from thermocycler. Briefly spin down PCR plate to collect droplets.
Vortex IPB to resuspend the beads.
Add 30 ul of IPB beads to each well.
Mix the total reaction volume by gently pipetting at least 10 times and incubate for 2 minutes at
room temperature.
Place on the magnetic stand and wait until the liquid is clear (~5 minutes)
Remove and discard all supernatant
Wash beads as follows: a. Keep on the magnetic stand and add 180 μl fresh 80% EtOH to each
well; b. Wait 30 seconds; and c. Remove and discard all supernatant
Repeat wash for a total of 2 washes.
Briefly spin for 10 seconds and return plate to magnet.
With a 20 μl pipette, remove all residual EtOH.
Dry on magnet for 2 minutes.
Remove from the magnetic stand and add 25 μl RSB to each well.
Pipette mix and incubate at room temperature for 2 minutes.
Place on the magnetic stand and wait until the liquid is clear (~2 minutes).
Transfer 24 μl supernatant to a new plate.
Keep on ice for same day use or store at −20° c. (SAFE STOPPING POINT).
Determine the concentration of each purified recovery product with the Qubit dsDNA HS Assay
Kit. Use 1-2 μl of sample DNA per measurement.
Proceed with 50 ng to BLT prep (next tab)

### Example 7—Enriching for Short Fragments

A workflow was performed as described in Example 1, and included an enrichment step for short fragments. The workflow included: high molecular weight tagmentation; mutagenesis PCR; library normalization; bottlenecking (suppression) PCR; library preparation; fragment analysis of products; and sequencing. The additional enrichment step for short fragment enrichment of certain fragments was performed on the products of the library preparation step. An example overview and timeline for short fragment enrichment in the workflow are depicted in FIG. 28 and FIG. 29, respectively. Of course, other workflows for such short fragment enrichment are also contemplated.

The enrichment step included hybridizing the products with selection probes, capturing the products hybridized to the selection probes with bead-linked capture probes, and amplifying the captured products. The enrichment step was substantially the same as that performed in Example 6.

### Example 8—Selection of Probes for Under-Represented Genomic Regions

This example relates to the generation and use of selection probes to enrich for regions of the genome including those which are typically under-represented in whole genome sequencing methods. Such regions of the genome may provide sequencing data that are systematically impacted by lower quality—such as elevated error rates, low mapping quality or depth anomalies—may fail to deliver consistently accurate variant calls even for SNVs and indels. Many of the reference characteristics that cause these systematic errors are well known, such as, highly repetitive regions have poor mapping quality and homopolymers are known to result in low base accuracy. This knowledge has been used to classify the genome into ‘easy’ and ‘difficult’ regions (Krusche P, et al. Nat Biotechnol. 2019; 37:555-560). Though these classifications can be helpful, they are not a perfect representation of the actual performance within these regions. For example, a large segmental duplication may be comprised of regions of high and low similarity, leading to very different variant calling accuracy.

Selection probes for use to enrich for targets in long fragments were identified by methods which included: selecting target regions of the genome; designing a probe set within the target regions; and identifying suitable probes within the probe set to generate a panel of selection probes. Briefly, for a panel that was focused on under-represented regions of the genome, target regions included those with low mappability (non-unique) portions of protein-coding regions, including introns and untranslated regions (JTRs). Such regions included those having a low mapping quality score (MAPQ) score, less than 50, in the HG38 reference sequence of the human genome; and those represented in the RefSeq database which includes non-redundant, well-annotated set of sequences, including genomic DNA and transcripts. MAPQ scores quantify the probability that a sequencing read is misplaced in a reference sequence (Li H, et al. (2008) Genome Research 18:1851-8). More regions of interest included certain groups of genes and regions of the genome. Examples included: (i) protein coding regions with low mappability, e.g., low mappability RefSeq sequences; (ii) CMRG genes, e.g., Wagner J., et al., (2022) Nat Biotechnol. 40: 672-680; (iii) deCODE Icelandic dark matter regions; (iv) regions with short tandem repeats (STR), e.g., Stevanovski I., et al., Sci Adv. 2022 March; 8(9): eabm5386; (v) MHC locus; (vi) ACMG genes; (vii) regions reported in Wenger A. M., et al., (2019) Nat Biotechnol. 37:1155-1162; and (viii) PGX genes.

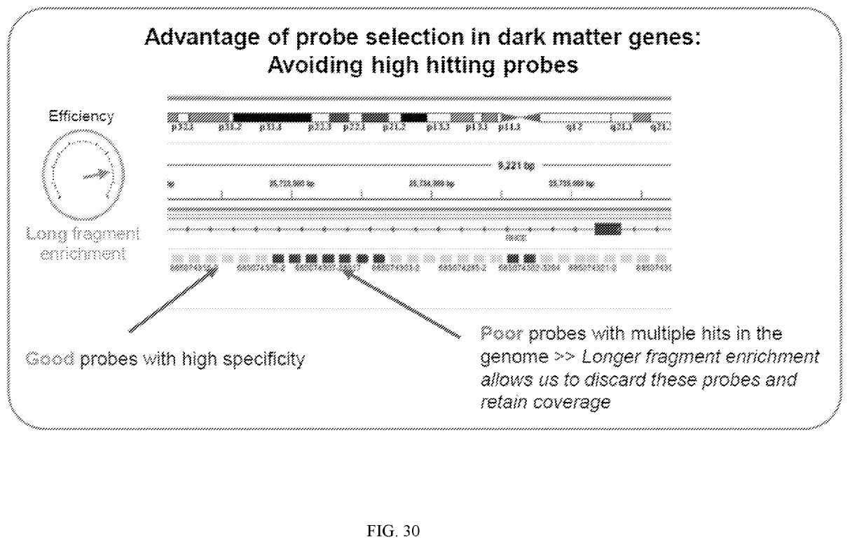

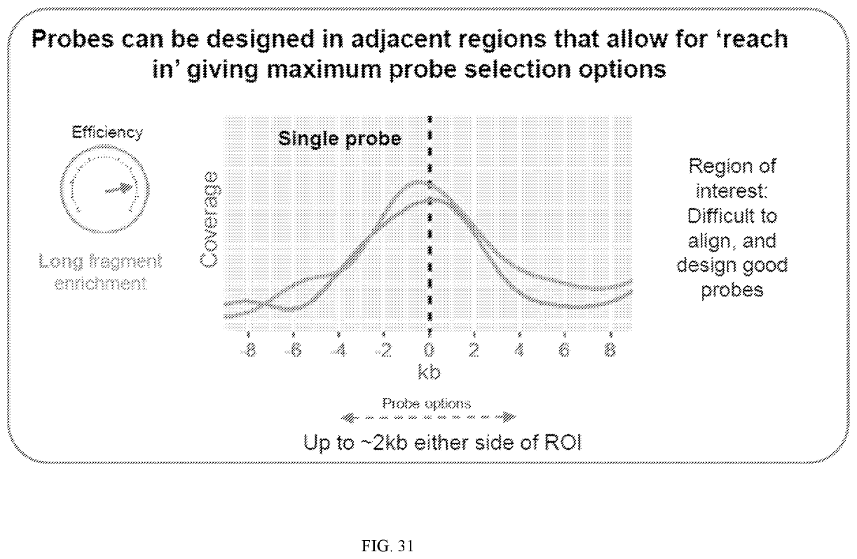

Probe sets were designed to hybridize within such target regions using software tools including DESIGNSTUDIO (Illumina, Inc., San Diego). Probes were designed to hybridize to target regions having about 1 kb+/−0.2 kb between corresponding sequences in the linear reference sequence. A 400 bp window centered around the 1 kb target spacing was searched. Within the 400 bp window, probes were identified using several parameters including GC bias (e.g. <25% to <75%) and uniqueness or hits in the genome. Suitable probes were predicted to hybridize or to hit less than 20 other regions of the reference genome. A hit was at least 80 consecutive bps of the 120 bp probes match >90%. 198,451 probes were identified. As depicted in FIG. 30, selection probes were identified having increased specificity for a target fragment.

In an example selection process for a fixed panel, probes were selected using the following steps: (1) Target regions were selected based on several inputs, including focusing on low mappability (non-unique) portions of protein-coding regions (including introns and JTRs), and certain identified groups of genes and genomic regions including: A CMG genes, CMRG genes, PGX genes, MHC locus, deCODE Icelandic dark matter regions. (2) Probes were designed to capture the above regions, aiming for about 1000 bp spacing between probes with the following sub-steps (2A)-(2D). (2A) To select better probes, 500 bp spacing was initially used. This provided two opportunities to find good probes for each region, which was useful to maximize the power of the data in a limited time. (2B) To achieve 1000 bp spacing, the algorithm jumped from one probe to the next in 1000 bp increments, but allowed a window of +/−200 bp to find the ‘best’ probe. (2C) There were multiple parameters within that window to find the ‘best’ probe. Mostly these were typical parameters such as CT content. However, a primary parameter for the panel was number of hits in the genome (minimize this number), and requirements on this parameter were relaxed to permit selection of probes in all regions. (2D) Probes were manufactured as double stranded DNA 80-mers or 120-mers. (3) At least 2 probes were selected per region, even for ‘small’ regions (e.g. 1000 bp, where 1 probe may target based on the above). (4) After probe selection was complete, additional rounds of selection were performed, based on the following sub-steps (4A)-(4D). (4A) Discarding region types that did not belong in the panel, for example, regions that were not low mappability portions of protein-coding regions. (4B) Discarding regions that were identified as “failed to cover” using the following sub-steps. (41B)(i). Regions that “failed to cover” included those that neither of the two panel designs covered at least half of the region 5×. This was calculated from the median of many samples, each with mean coverage in the 30× range. (4B)(ii) There were two designs for this panel which provided a choice of which probes to keep. ‘Panel A’ was kept as a default, because it had more replicates and typically saw better coverage overall, and slightly more probes were in this panel. ‘Panel B’ was kept if panel B gave at least 20% better coverage (calculated by fraction of bases covered >10×). (4C) To be even more rigorous about probes that hit multiple times in the genome, all probes were discarded that hit at least 20 times elsewhere in the genome. A hit included at least 50 consecutive bps of the 120 bp probes match >90%. This resulted in some gaps larger than desired. If there was a gap:>1.5 kb or a region with a single probe, the other panel was examined probes from the other panel were added to cover those gaps. (4D) Any corrections that were needed were then made to align probes with coordinates of genomic sequences.

Additional “spike in” probes were identified in target regions using the following steps. Gaps between probes were identified in the remaining target regions that were larger than 1.5 kb, and target regions were identified that had gaps of larger than 1.5 kb between the first or last remaining probe and the target region start or stop location on the chromosome. For each identified 1.5 kb gap, unselected probes which targeted the region from panel A or panel B were identified and selected to fill the gap, making sure that such selected probes did not bind excessively (more than 20 times) in other parts of the genome. Separately, target regions were identified that were left with a single probe after going through the previous selection criteria. Targets could be left with a single probe because all the other probes assigned to that target region were liable to bind to >20 other places in the genome and had been removed, or because many target regions only had one probe from each uber panel A and uber panel B and only one of the panel batches to use for those targets had been picked. For these single-probe targets, unpicked probes were added that hit that same target from the non-picked panel, provided that those probes were also not labeled to bind excessively in other parts of the genome and that those probes had also met the same coverage criteria in the previous steps. Initial probe panels including ‘Uber A’ and ‘Uber B’ were generated. Probes within these panels were excluded with criteria that included having at least 20 hits in the genome, to generate a ‘final’ panel. TABLE 2A summarizes steps used to generate different batches of selections probes using steps described above.

TABLE 2A

| Step included | Batch | | | | |
|---|---|---|---|---|---|
| in batch | | | Uber A and | First | Second |
| generation | Final | Spike-in | Uber B | order | order |
| (1) | Yes | Yes | Yes | Yes | Yes |
| (2A) | Yes | Yes | Yes | Yes | Yes |
| (2B) | Yes | Yes | Yes | Yes | Yes |
| (2C) | Yes | Yes | Yes | Yes | Yes |
| (2D) | Yes | Yes | Yes | Yes | Yes |
| (3) | Yes | Yes | Yes | Yes | Yes |
| (4A) | Yes | Yes | No | No | No |
| (4B)(i) | Yes | Yes | No | No | No |
| (4B)(ii) | Yes | Yes | No | No | No |
| (4C) | Yes | No | No | No | No |
| (4D) | Yes | Yes | No | No | No |

TABLE 2B lists certain probes generated by the foregoing methods.

TABLE 2B

| | Characteristic | | |
|---|---|---|---|
| | Size | Approx spacing | No of |
| Batch | (bases) | in genome (kb) | probes |
| Final | 120 | 1 kb | 39953 |
| Uber A | 120 | 1 kb | 81325 |
| Spike-in | 120 | 1 kb | 1491 |

A series of selection probe panels were developed using methods substantially similar to the foregoing methods. Panels included: (1a) uber A: (1b) uber B; (2) intermediate/dev; and (3) final. Panels (1)-(3) were progressive iterations of a selection probe panel. The panels were used to prepare libraries with a method with long fragments with enrichment, and substantially similar to the WGS with enrichment workflow depicted in FIG. 32. A control included a library prepared with a method with long fragments, without enrichment, substantially similar to the WGS workflow depicted in FIG. 32. TABLE 2C lists certain metrics for data obtained using each method/panel which show that in methods that included enrichment, compared to methods without enrichment, median coverage levels and N50 values were much greater despite a smaller number of paired end reads.

TABLE 2C

| | With enrichment | | | | | Without |
|---|---|---|---|---|---|---|
| | Panel | Panel | Panel | Panel | Panel | enrichment |
| Metric | (1a) | (1a) | (1b) | (2) | (3) | None |
| Level of input reads | Low | High | High | Low | Low | High |
| (# paired end reads) | 1.0E+09 | ~5E+09 | ~3E+09 | 6.0E+08 | 5.9E+08 | 5.4E+09 |
| target region size (Mbp) | 76 | 76 | | 40 | 40 | N/A |
| median coverage | 32.2 | 72.2 | 67.5 | 34.4 | 41.9 | 27.3 |
| percent bases with no | 5.2% | 2.3% | 3.7% | 2.3% | 2.4% | 2.0% |
| coverage | | | | | | |
| percent bases with less | 20.1% | | | 15.3% | 12.3% | 11.5% |
| than 10x coverage | | | | | | |
| N50 (bp) | 6089 | 4198 | 4192 | 6677 | 7116 | 7054 |
| percent reads on target | 72% | | | 85% | 85% | N/A |
| f-measure non-SNP type 2 | 0.737 | 0.784 | 0.732 | 0.841 | 0.841 | 0.881 |
| f-measure SNP type 2 | 0.885 | 0.902 | 0.882 | 0.905 | 0.911 | 0.947 |
| precision non-SNP type 2 | 0.841 | 0.878 | 0.847 | 0.848 | 0.869 | 0.889 |
| precision SNP type 2 | 0.923 | 0.92 | 0.892 | 0.952 | 0.947 | 0.96 |
| sensitivity non-SNP type 2 | 0.656 | 0.709 | 0.644 | 0.834 | 0.814 | 0.874 |
| sensitivity SNP type 2 | 0.849 | 0.884 | 0.872 | 0.863 | 0.879 | 0.934 |

The data in TABLE 2C show that in methods that included enrichment, compared to methods without enrichment, median coverage levels and N50 values were much greater despite a smaller number of input paired end reads.

### Example 9—Major Histocompatibility Complex (MHC) Panel

The MHC region is a large locus located on the short arm of human chromosome 6 (6p21.1-6p21.3), and contains highly polymorphic genes that code for cell surface proteins, essential for the adaptive immune system. The region is challenging to obtain sequence information due to presence of high level of repetitive sequences, sequence homology, pseudo genes, and a wide variety of alleles in the population. Precise genotyping and phasing of the MHC region is challenging but highly clinically desirable for applications like organ transplantation and drug discovery.

Panels of selection probes were prepared which targeted approximately 5 Mbp of the MHC region. A first panel of selection probes was designed such that probes were spaced about 230 bp from one another on the HG38 reference sequence of the MHC region. Probe sequences were designed to hybridize within the target region using software tools, DESIGNSTUDIO (Illumina, Inc., San Diego). Potential probe sequences were evaluated by several criteria including number of hits within the genome, and percentage GC content. Probes having more than about 20 hits within the genome were discarded. A second panel of selection probes was prepared such that probes would be spaced about 1 kb from one another on a reference sequence of the MHC. The second panel was prepared by removing probes from the first panel. Probes included 80-mers and 120-mers. A batch of clinical probes was designed by a method including: targeting a 1 kb window in a reference sequence; identifying potential probes within the 1 kb using software tools, DESIGNSTUDIO (Illumina, Inc., San Diego); selecting a probe within the 1 kb based on criteria including less than 20 hits within the reference genome, and GC content. A hit included probes having at least 90% sequence identity along the full length of the probe with a target complement in the reference sequence. TABLE 3A lists probes generated by the foregoing methods.

TABLE 3A

| | Oligo characteristic | | | |
|---|---|---|---|---|
| | Size | Approx spacing | | No of |
| Batch | (bases) | in genome | | probes |
| Clinical | 120 | 1 | kb | 4966 |
| Final design | 120 | 1 | kb | 5153 |
| Final design | 80 | 1 | kb | 5155 |
| Input4_120_designs | 120 | 230 | bp | 21608 |
| Input4_120_designs | 80 | 230 | bp | 21608 |

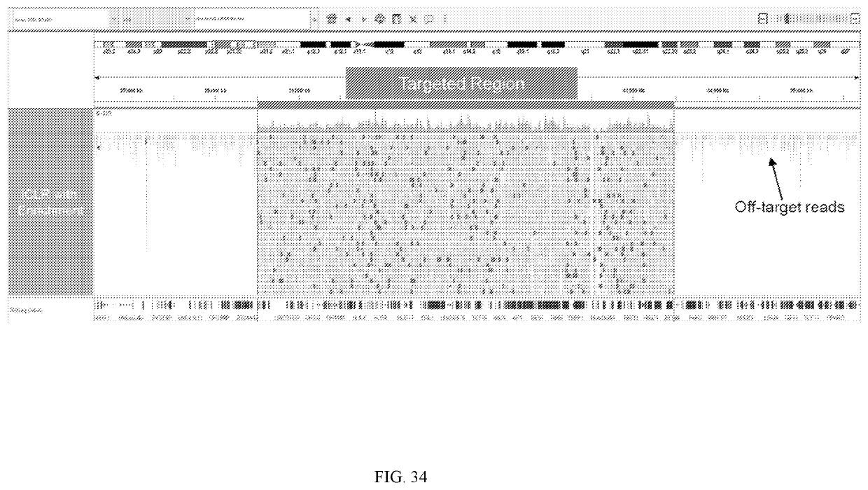

The panels were used to prepare libraries with a method with long fragments and enrichment, and substantially similar to the WGS with enrichment workflow depicted in FIG. 32. Use of the MHC probe panel generated highly specific and efficient enrichment over the target region, with 98.3% long-reads on-target. FIG. 34 depicts an example of coverage for the targeted region. TABLE 3B lists certain sequencing metrics.

TABLE 3B

| Parameter | MHC panel |
|---|---|
| Read N50 | 5932 |
| Error Rate | 0.71% |
| Mean Coverage | 43.05 |
| %0 Coverage | 1.06% |
| % Low Coverage (1-3X) | 0.64% |
| % On Target Reads | 98.3% |

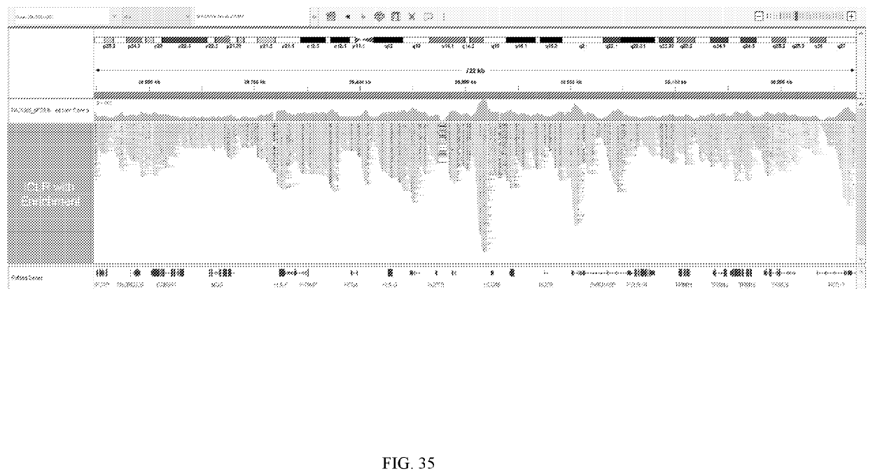

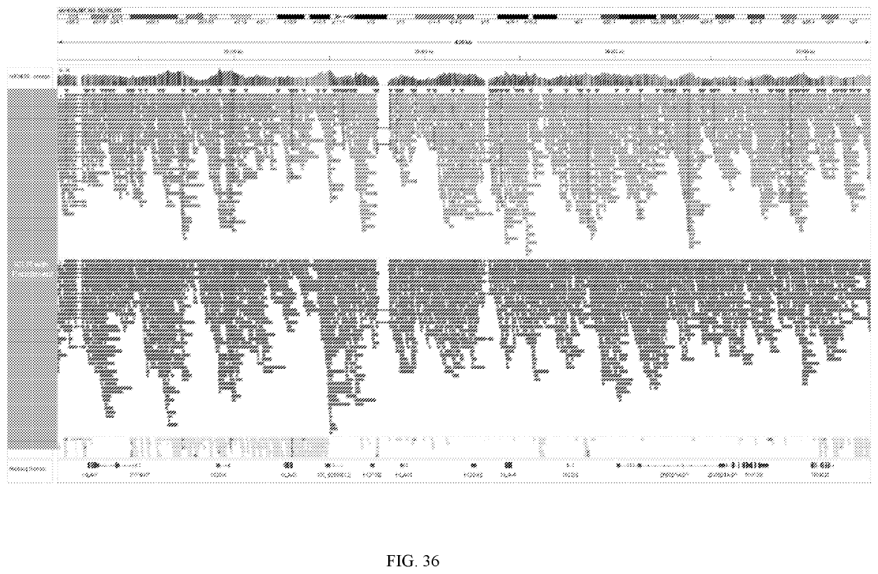

Use of the MHC selection probe panel resolved haplotype in polymorphic genes. Phasing worked well with use of the MHC probe panel in methods with long fragments and enrichment (e.g. WGS with enrichment workflow depicted in FIG. 32), and was comparable to such methods without enrichment (e.g. WGS without workflow depicted in FIG. 32). TABLE 3C lists certain sequencing metrics. As depicted in FIG. 35, a 722 kb region in the MHC locus was analyzed, and a 580 kb region was encapsulated in one phase block. As depicted in FIG. 36, a 426 kb region in the MHC locus that covered HLA-A, HLA-G, HLA-F, was fully phased.

TABLE 3C

| Parameter | MHC panel |
|---|---|
| % Biallelic Het SNVs Phased | 95.88% |
| Block N50 | 274,215 |
| Switch Error Rate | 0.14% |

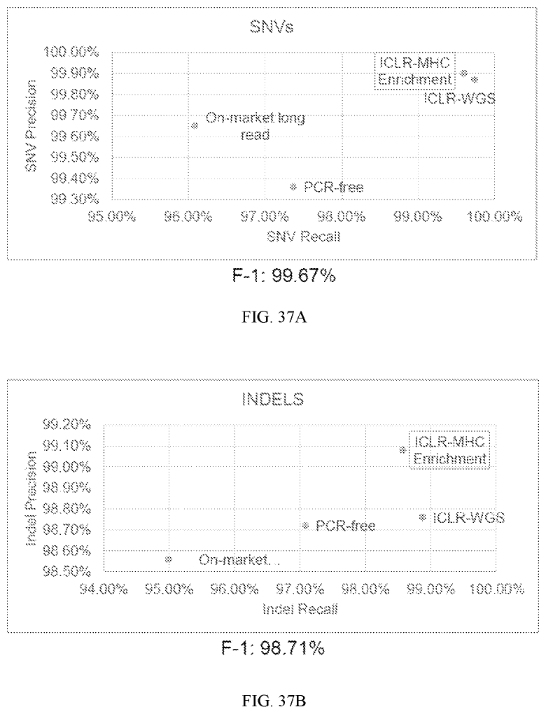

Use of the MHC selection probe panel achieved accurate variant calling in the MHC region. FIG. 37A depicts a graph for SNV precision vs SNV recall for sequence information obtained from nucleic acid libraries prepared by (1) on market long read kits; (2) short read (SR) PCR-free sequencing methods, such as those including simple tagmentation of gDNA; (3) method with long fragments and enrichment, such as the WGS with enrichment workflow depicted in FIG. 32 (1CLR-MHC enrichment); and (4) method with long fragments without enrichment, such as the WGS workflow depicted in FIG. 32 (ICLR-WGS). Use of the MHC panel with long fragment with enrichment methods achieved levels of SNV precision greater than methods without enrichment.

FIG. 37B depicts a graph for INDEL precision vs INDEL recall for sequence information obtained from nucleic acid libraries prepared by (1) on market long read kits; (2) PCR-free methods; (3) method with long fragments and enrichment, such as the WGS with enrichment workflow depicted in FIG. 32 (ICLR-MHC enrichment); and (4) method with long fragments without enrichment, such as the WGS workflow depicted in FIG. 32 (ICLR-WGS). Use of the MHI C panel with long fragment with enrichment methods achieved levels of indel precision greater than methods without enrichment.

### Example 10—American College of Medical Genetics (ACMG) Panel

A selection probe panel was generated from a list of genes compiled by ACMG for which specific mutations are known to be causative of disorders that are clinically actionable. The panel was designed to precisely call variants and phase in these genes. The panel included 78 unique genes in ACMG SF v3.1 and targeted full genes. The panel size was about 6.8 Mbp. The ACMG genes included those listed in TABLE 1A.

Panels of selection probes were prepared which targeted the ACMG genes. A first panel of selection probes was designed such that probes were spaced about 230 bp from one another on the HG38 reference sequence of the ACMG genes. Probe sequences were designed to hybridize within the target region using software tools, DESIGNSTUDIO (Illumina, Inc., San Diego). Potential probe sequences were evaluated by several criteria including number of hits within the genome, and percentage GC content. Probes having more than about 20 hits within the genome were discarded. A second panel of selection probes was prepared such that probes would be spaced about 1 kb from one another on a reference sequence of the ACMG genes. The second panel was prepared by removing probes from the first panel. Probes included 80-mers and 120-mers. A batch of clinical probes was designed by a method including: targeting a 1 kb window in a reference sequence; identifying potential probes within the 1 kb using software tools, DESIGNSTUDIO (Illumina, Inc., San Diego); selecting a probe within the 1 kb based on criteria including less than 20 hits within the reference genome, and GC content. A hit included probes having at least 90% sequence identity along the full length of the probe with a target complement in the reference sequence. TABLE 4A lists probes generated by the foregoing methods.

TABLE 4A

| | Characteristic | | | |
|---|---|---|---|---|
| | Size | Approx spacing | | No of |
| Batch | (bases) | in genome | | probes |
| Clinical 1 kb | 120 | 1 | kb | 6935 |
| 120mer_1 kb_Optimized- | 120 | 1 | kb | 7513 |
| Final | | | | |
| 80mer_1 kb_Optimized- | 80 | 1 | kb | 7520 |
| Final | | | | |
| Clinical 230 bp | 120 | 230 | bp | 29954 |
| ACMG_120mer_designs | 120 | 230 | bp | 29954 |
| ACMG_80mer_designs | 80 | 230 | bp | 29954 |

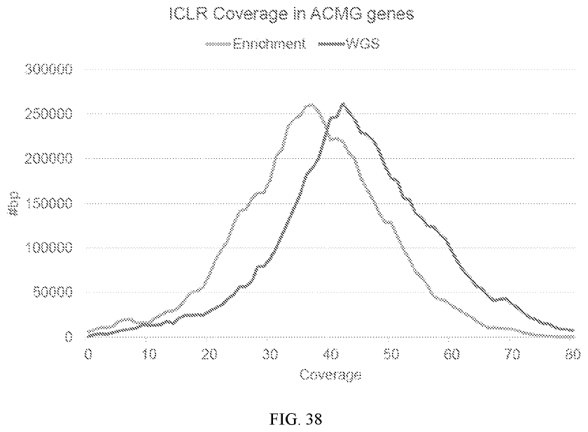

Use of the ACMG selection probe panel was highly effective and achieved comparable coverage performance with long read whole genome sequencing methods without the use of the panel (FIG. 38) TABLE 4B lists certain sequencing metrics

TABLE 4B

| Parameter | ACMG panel |
|---|---|
| Read N50 | 7497 |
| Error Rate | 0.26% |
| Mean Coverage | 59.47 |
| %0 Coverage | 0.04% |
| % Low Coverage (1-3X) | 0.22% |
| % On Target Reads | 87.4% |

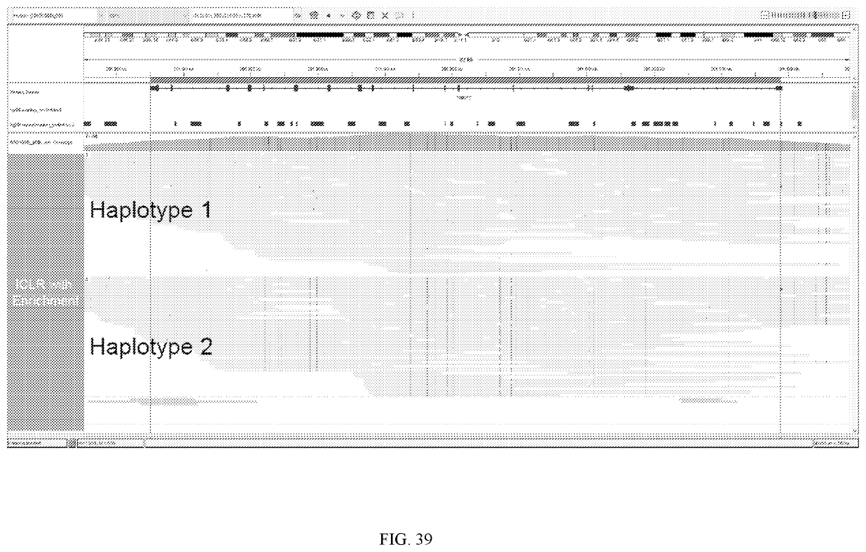

99.5% of heterozygous SNVs were phased using the ACMG panel. For example, as depicted in FIG. 39, TNNT2, a 22 kb gene was fully phased in one phase block. TABLE 4C lists certain sequencing metrics. Phasing metrics were in target genes only, hence a shorter block N50.

TABLE 4C

| Parameter | ACMG panel |
|---|---|
| % Biallelic Het SNVs Phased | 99.5% |
| Block N50 | 85841 |
| Switch Error Rate | 0.17% |

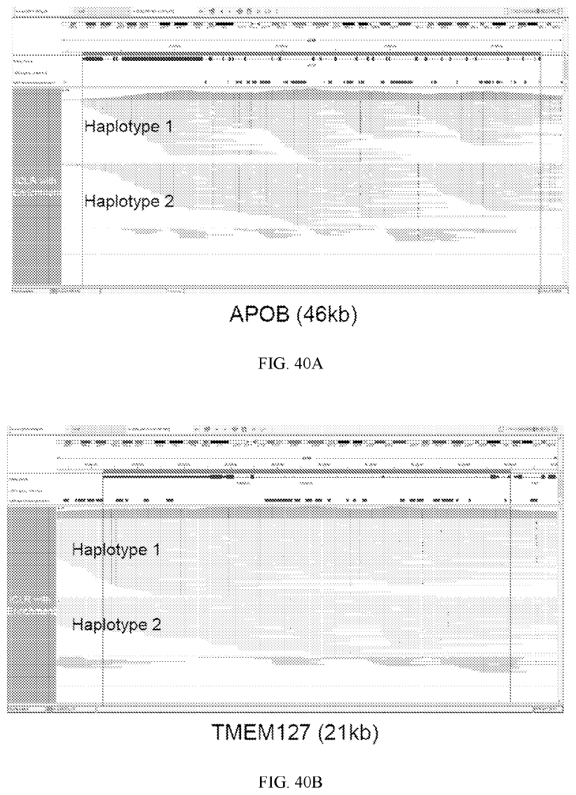

99% of heterozygous SNVs were phased using the ACMG panel. For example, as depicted in FIG. 40A for APOB and in FIG. 40B for TMEM127, genes were fully phased in one phase block.

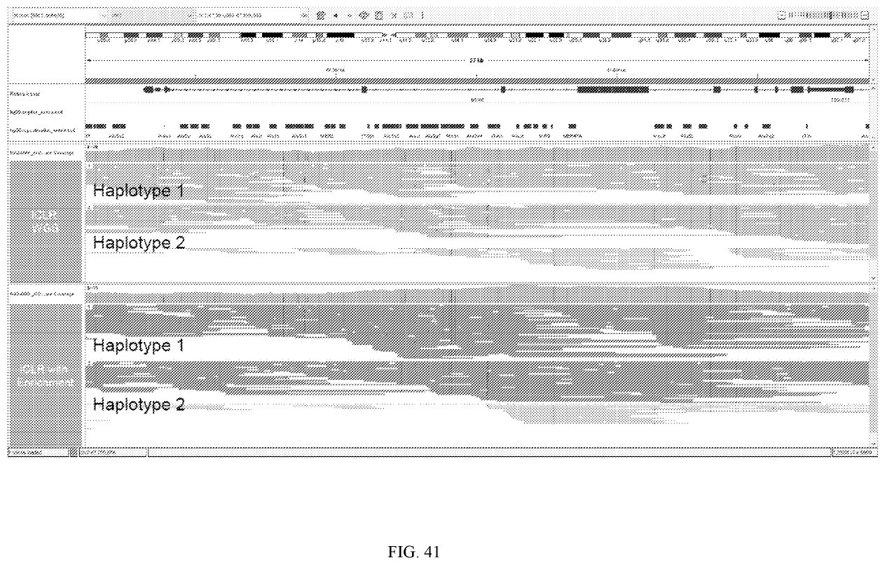

Use of the ACMG panel covered and phased highly repetitive MSH6 (70,76% contains repeat elements), and enrichment achieved comparable coverage and phasing compared to long read whole genome sequencing methods without the use of the panel (FIG. 41), 27 kb was fully encapsulated in one phase block. Use of the ACMG panel covered and phased highly repetitive NF2 (45% contains repeat elements), and enrichment achieved comparable coverage and phasing compared to long read whole genome sequencing methods without the use of the panel. 99 kb was fully encapsulated in one phase block.

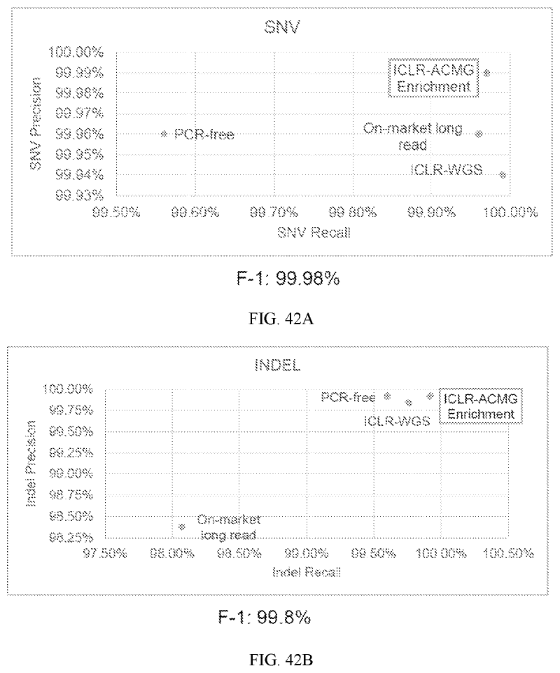

Use of the ACMG selection probe panel achieved highly accurate variant calling in the ACMG. FIG. 42A depicts a graph for SNV precision vs SNV recall for sequence information obtained from nucleic acid libraries prepared by (1) on market long read kits; (2) PCR-free methods; (3) method with long fragments and enrichment, such as the WGS with enrichment workflow depicted in FIG. 32 (ICLR-ACMG enrichment); and (4) method with long fragments without enrichment, such as the WGS workflow depicted in FIG. 32 (ICLR-WGS). Use of the ACMG panel with long fragment with enrichment methods achieved levels of SNV precision greater than methods without enrichment. FIG. 42B depicts a graph for INDEL precision vs INDEL recall for sequence information obtained from nucleic acid libraries prepared by (1) on market long read kits; (2) PCR-free methods; (3) method with long fragments and enrichment, such as the WGS with enrichment workflow depicted in FIG. 32 (ICLR-ACMG enrichment); and (4) method with long fragments without enrichment, such as the WGS workflow depicted in FIG. 32 (ICLR-WGS). Use of the ACMG panel with long fragment with enrichment methods achieved levels of indel precision and indel recall greater than methods without enrichment.

### Example 11—Pharmacogenetic (PGX) Panel

A selection probe panel was generated for genes commonly targeted by pharmacogenetic testing assays. Genetic variation is known to influence the way individual respond to therapeutics. Accurately detecting functional haplotypes (“star alleles”) in clinically actionable pharmacogenetic genes is crucial to implementation of personalized medicine. The panel was generated to achieve highly accurate genotyping and star allele calling in such genes. The panel included 98 genes that are important in pharmacogenetics, targeting full genes. The panel size was about 8.1 Mbp. The genes included those listed in TABLE 1B.

Panels of selection probes were prepared which targeted the PGX genes. A first panel of selection probes was designed such that probes were spaced about 230 bp from one another on the HG38 reference sequence of the PGX genes. Probe sequences were designed to hybridize within the target region using soft are tools, DESIGNSTUDIO (Illumina, Inc., San Diego). Potential probe sequences were evaluated by several criteria including number of hits within the genome, and percentage GC content. Probes having more than about 20 hits within the genome were discarded. A second panel of selection probes was prepared such that probes would be spaced about 1 kb from one another on a reference sequence of the PGX genes. The second panel was prepared by removing probes from the first panel. Probes included 80-mers and 120-mers. A batch of clinical probes was designed by a method including: targeting a 1 kb window in a reference sequence; identifying potential probes within the 1 kb using software tools, DESIGNSTUDIO (Illumina, Inc., San Diego); selecting a probe within the 1 kb based on criteria including less than 20 hits within the reference genome, and GC content. A hit included probes having at least 90% sequence identity along the full length of the probe with a target complement in the reference sequence. TABLE 5A lists probes generated by the foregoing methods.

TABLE 5A

| | Characteristic | | | |
|---|---|---|---|---|
| | Size | Approx spacing | | No of |
| Batch | (bases) | in genome | | probes |
| Clinical 1 kb | 120 | 1 | kb | 8153 |
| PGX_80mer_1 kb | 80 | 1 | kb | 8693 |
| PGX_80mer_230 bp | 80 | 230 | bp | 35205 |
| PGX_80mer_230bp_v1 | 80 | 230 | bp | 33089 |

TABLE 5B lists certain sequencing metrics for methods with long fragments and enrichment with the PGX panel, and shows highly effective target region coverage.

TABLE 5B

| Parameter | PGx panel |
|---|---|
| Read N50 | 7279 |
| Mean Coverage | 57.17 |
| %0 Coverage | 0.12% |
| % Low Coverage (1-3X) | 0.32% |
| % On Target Reads | 88.3% |

Star Alleles are a nomenclature system used to describe allelic variation. In the case of PGX genes, star alleles describe haplotype patterns associated with protein-level interactions, with * 1 usually referring to the wild-type or “fully-functional” haplotype. TABLE 5C lists star alleles called by methods which include long fragments, and enrichment (ICLR PGX enrichment) which were 100% concordant with on market long read methods.

TABLE 5C

| Gene | ICLR PGx Enrichment | On-market Long Reads |
|---|---|---|
| CYP2C19 | *1.002, *1.002 | *1.002, *1.002 |
| CYP2C8 | *1.001, *1.004 | *1.001, *1.004 |
| CYP2C9 | *1.001, *1.001 | *1.001, *1.001 |
| CYP2E1 | *1.002, *7.002 | |
| CYP2J2 | *1.001, *1.001 | *1.001, *1.001 |
| CYP3A4 | *1.002, *1.002 | *1.002, *1.002 |
| CYP3A5 | *3.001, *3.001 | *3.001, *3.001 |
| CYP3A7 | *1.001, *1.001 | *1.001, *1.001 |
| CYP4F2 | *1.001, *4.1001 or | *1.001, *4.1001 or |
| | *2.001, *3.001 | *2.001, *3.001 |
| NUDT15 | *1.001, *1.001 | *1.001, *1.001 |
| SLCO1B1 | *1.002, *1.002 | *1.002, *1.002 |
| TPMT | *1.001, *1.001 | *1.001, *1.001 |
| UGT1A1 | *1, *1 | *1, *1 |

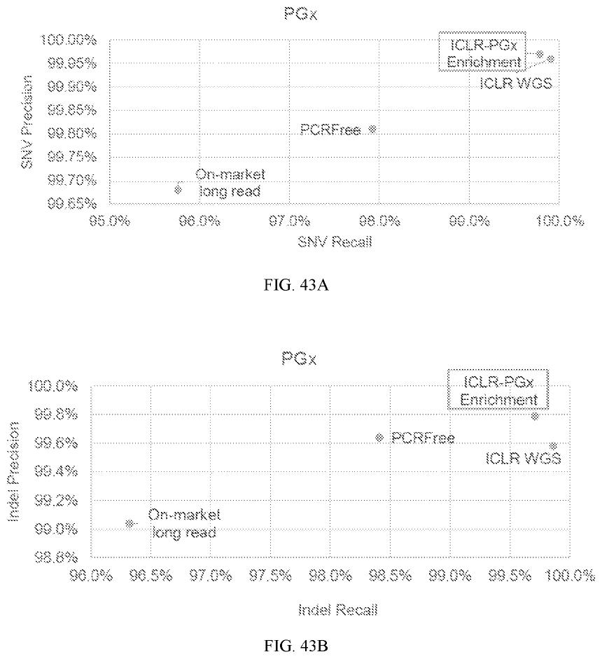

Use of the POX selection probe panel achieved highly accurate variant calling in the PGX genes. FIG. 43A depicts a graph for SNV precision vs SNV recall for sequence information obtained from nucleic acid libraries prepared by (1) on market long read kits; (2) PCR-free methods; (3) method with long fragments and enrichment, such as the WGS with enrichment workflow depicted in FIG. 32 (ICLR-PGX enrichment); and (4) method with long fragments without enrichment, such as the WGS workflow depicted in FIG. 32 (ICLR-WGS). Use of the PGX panel with long fragment with enrichment methods achieved levels of SNV precision and indel recall greater than methods without enrichment. FIG. 43B depicts a graph for INDEL precision vs INDEL recall for sequence information obtained from nucleic acid libraries prepared by (1) on market long read kits; (2) PCR-free methods: (3) method with long fragments and enrichment, such as the WGS with enrichment workflow depicted in FIG. 32 (ICLR-PGX enrichment); and (4) method with long fragments without enrichment, such as the WGS workflow depicted in FIG. 32 (ICLR-WGS). Use of the PGX panel with long fragment with enrichment methods achieved levels of indel precision and indel recall greater than methods without enrichment.

### Example 12—Challenging Medically Relevant Genes (CMRG) Panel

A selection probe panel was generated for genes that are medically relevant autosomal genes that are excluded from the GiaB v4.2.1 variant benchmark due to repeats or polymorphic complexities (Wagner J., et al, (2022) Nat Biotechnol. 40: 672-680). These genes have <90% bases included in the benchmark and pose a challenge for their accurate analysis in a clinical setting. The selection probe panel was generated to rescue coverage gap in these genes. The panel targeted 389 genes, with a panel size of 22.5 Mbp. The average overlap with repeat: 36.6%, and the average overlap with segdup was 19.7%. The genes include those listed in TABLE 1C.

Panels of selection probes were prepared which targeted the CMRG genes. A first panel of selection probes was designed such that probes were spaced about 230 bp from one another on the HG38 reference sequence of the CMRG genes. Probe sequences were designed to hybridize within the target region using software tools, DESIGNSTUDIO (Illumina, Inc., San Diego). Potential probe sequences were evaluated by several criteria including number of hits within the genome, and percentage GC content. Probes having more than about 20 hits within the genome were discarded. A second panel of selection probes was prepared such that probes would be spaced about 1 kb from one another on a reference sequence of the CMRG genes. The second panel was prepared by removing probes from the first panel. Probes included 80-mers and 120-mers. A batch of clinical probes was designed by a method including: targeting a 1 kb window in a reference sequence; identifying potential probes within the 1 kb using software tools, DESIGNSTUDIO (Illumina, Inc., San Diego); selecting a probe within the 1 kb based on criteria including less than 20 hits within the reference genome, and GC content. A hit included probes having at least 90% sequence identity along the full length of the probe with a target complement in the reference sequence. TABLE 6A lists probes generated by the foregoing methods.

TABLE 6A

| | Characteristic | | | | |
|---|---|---|---|---|---|
| | | Approx | | | |
| | Size | spacing | | No of | |
| Batch | (bases) | in genome | | probes | SEQ ID NOs. |
| Clinical | 120 | 1 | kb | 22545 | 02-22546 |
| CMG_80mer_1 kb | 80 | 1 | kb | 21333 | 22547-43879 |
| CMG_80mer_230 bp | 80 | 230 | bp | 96721 | 43880-140600 |

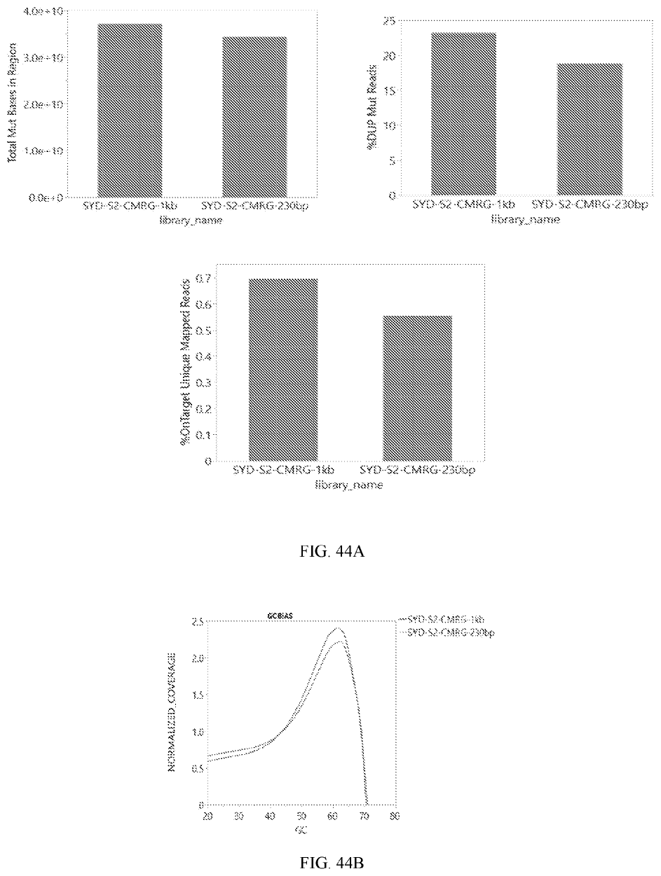

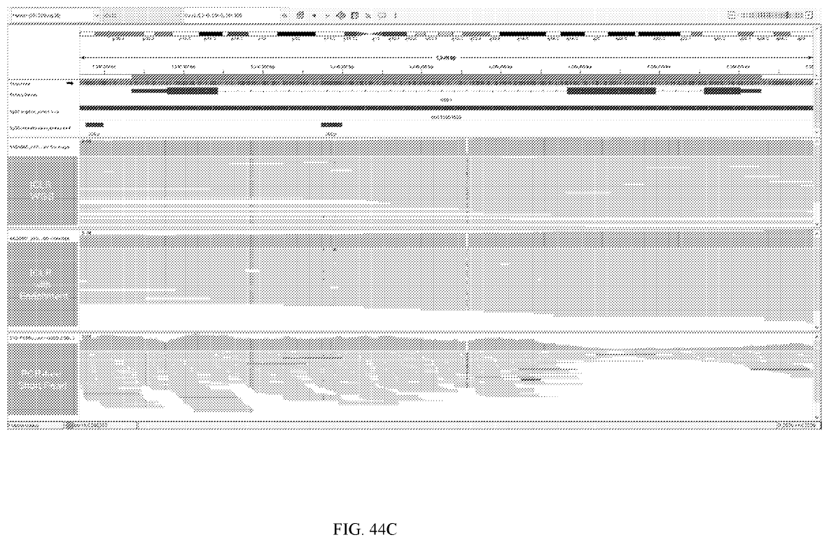

Methods using long fragments with enrichment with either the first selection panel (230 bp spacing), or the second selection panel were compared. As depicted in FIG. 44A, use of the second selection probe panel (SYD-C2-CMRG-1 kb) compared to use of the first selection probe panel (SYD-C2-CMRG-230 bp), resulted in increased identification in sequence data for (i) total mutations in bases in region; (ii) percentage DUP mutant reads; and (iii) percentage on target unique mapped reads. FIG. 44B compares normalized coverage between use of the first selection probe panel (SYD-C2-CMRG-230 bp) and the second selection probe panel (SYD-C2-CMRG-1 kb).

Coverage of certain genes, including CFC1, HBG1, OR51A2, RGPD3, and CYP4F3 was compared using methods with long fragment and enrichment (ICLR with enrichment), methods with long fragments without enrichment (ICLR WGS), and PCR free short reads (SR). FIG. 44C depicts an example with respect to HBG1 and shows that enrichment with the CMRG panel rescued a SR coverage dip in the gene. With regard to other genes, for CFC1, enrichment with the CMRG panel increased coverage in the gene. For HBG1, enrichment with the CMRG panel rescued a SR coverage dip in the gene. For OR51A2, enrichment with the CMRG panel rescued a SR coverage gap in the gene. For the RGPD3 gene, enrichment with the CMRG panel rescued an SR coverage dip in the gene. For the CYP4F3 gene, enrichment with the CMRG panel resolved SVs missed by SR in the gene.

TABLE 6B lists certain metrics which shows that methods with long fragments and enrichment with the CMRG panel achieved highly effective target region coverage.

TABLE 6B

| Parameter | CMRG panel |
|---|---|
| Read N50 | 6432 |
| Mean Coverage | 47.68 |
| %0 Coverage | 2.74% |
| % Low Coverage (1-3X) | 0.92% |
| % On Target Reads | 80.5% |

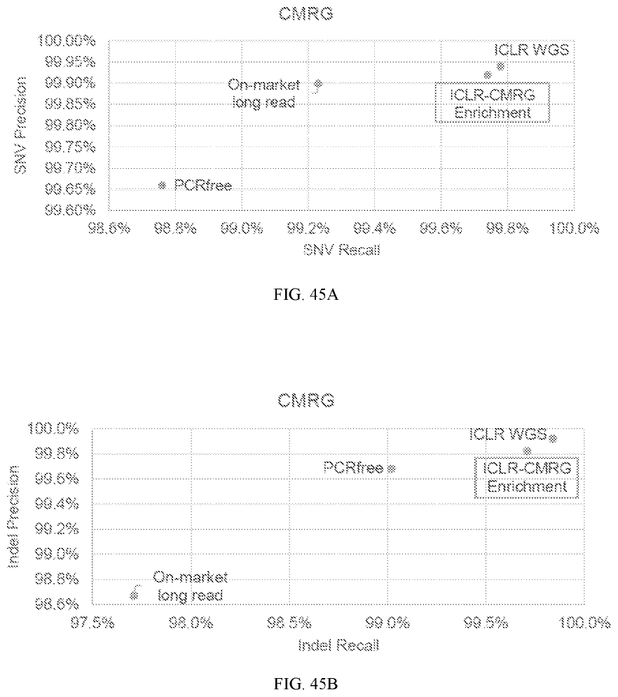

Use of the CMRG selection probe panel achieved highly accurate variant calling in the CMRG genes. FIG. 45A depicts a graph for SNV precision vs SNV recall for sequence information obtained from nucleic acid libraries prepared by (1) on market long read kits; (2) PCR-free methods; (3) method with long fragments and enrichment, such as the WGS with enrichment workflow depicted in FIG. 32 (ICLR-CMRG enrichment); and (4) method with long fragments without enrichment, such as the WGS workflow depicted in FIG. 32 (ICLR-WGS). FIG. 45B depicts a graph for INDEL precision vs INDEL recall for sequence information obtained from nucleic acid libraries prepared by (1) on market long read kits; (2) PCR-free methods; (3) method with long fragments and enrichment, such as the WGS with enrichment workflow depicted in FIG. 32 (ICLR-CMRG enrichment); and (4) method with long fragments without enrichment, such as the WGS workflow depicted in FIG. 32 (ICLR-WGS).

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1.-101. (canceled)

102. A method for preparing a nucleic acid library, comprising:
(a) obtaining a plurality of transposomes comprising transposon adaptors, wherein the plurality of transposomes is immobilized on a plurality of beads;
(b) contacting a plurality of nucleic acid fragments with the plurality of transposomes to obtain a plurality of polynucleotides;
(c) amplifying the plurality of polynucleotides to obtain amplified polynucleotides by:
(i) performing a mutagenesis PCR, such that mutations are introduced into amplified polynucleotides, and
(ii) performing a suppression PCR; and
(d) adding library adapters to each end of the amplified polynucleotides, thereby obtaining the nucleic acid library;
further comprising enriching for target polynucleotides in (i) the plurality of polynucleotide, (ii) the amplified polynucleotides, and/or (iii) nucleic acids of the nucleic acid library by hybridizing a plurality of selection probes to the target polynucleotide; wherein the selection probes of the plurality of selection probes comprise at least 50 different nucleotide sequences;
wherein each selection probe of the plurality of selection probes comprises a nucleotide sequence capable of hybridizing to a gene selected from TABLE 1C; and/or a nucleotide sequence within no more than 10 kbp 5′ or no more than 10 kbp 3′ of a gene in a human genome selected from TABLE 1C.

103. The method of claim 102, wherein each selection probe of the plurality of selection probes comprises a nucleotide sequence having at least 90% sequence identity to any one of SEQ ID NOs:02-140601.

104. The method of claim 102, wherein the plurality of selection probes is attached to a substrate.

105. The method of claim 102, further comprising amplifying the target polynucleotides.

106. The method of claim 102, wherein the plurality of the transposomes is immobilized on the plurality of beads:
(i) at a density such that an average length of the plurality of polynucleotides is greater than about 1 kbp, and/or the average length of the plurality of polynucleotides is in a range from about 1 kbp to about 40 kbp;
(ii) such that the number of transposomes immobilized on the plurality of beads is no more than about 100 transposomes per bead;
(iii) comprise a total activity such that an average length of the plurality of polynucleotides is greater than about 1 kbp, and/or wherein the average length of the plurality of polynucleotides is in a range from about 1 kbp to about 40 kbp; and/or
(iv) comprise an activity in a range from about 0.05 AU/μl to about 0.25 AU/μl.

107. The method of claim 102, wherein the transposomes of the plurality of transposomes are the same.

108. The method of claim 102, wherein the mutagenesis PCR comprises: (i) amplifying the plurality of polynucleotides with a low bias DNA polymerase; (ii) amplifying the plurality of polynucleotides with a nucleotide analogue; and/or (iii) no more than 6 cycles.

109. The method of claim 102, wherein a first end of a polynucleotide of the plurality of polynucleotides is capable of annealing to a second end of the polynucleotide of the plurality of polynucleotides; and/or, wherein a first end of an amplified polynucleotide is capable of annealing to a second end of the amplified polynucleotide.

110. The method of claim 102, wherein the suppression PCR comprises (i) use of a single amplification primer; and/or (ii) no more than 6 cycles.

111. The method of claim 102, wherein step (d) comprises contacting the amplified polynucleotides with an additional plurality of transposomes comprising the library adapters.

112. A method for determining a sequence of a target nucleic acid, comprising:
performing the method of claim 102;
sequencing the nucleic acid library to obtain sequence reads; and
assembling sequence reads to obtain the sequence of a target nucleic acid.

113. The method of claim 112, wherein the assembling comprises determining mutations introduced into the amplified polynucleotides during the mutagenesis PCR and comparing the sequence reads to a reference sequence.

114. A kit comprising:
a first bead-linked transposomes (BLT-1) reagent, wherein the BLT-1 transposomes reagent comprises a first adaptor sequence;
a mutagenesis reagent comprising a first primer, dPTPs, dNTPs, and a polymerase;
a second bead-linked transposomes (BLT-2) reagent, wherein the BLT-2 transposomes reagent comprise the first adaptor and a second adaptor;
an amplification reagent comprising a first primer, a second primer, dNTPs, and a polymerase; and
a plurality of selection probes, wherein each selection probe of the plurality of selection probes comprises a nucleotide sequence capable of hybridizing to a gene selected from TABLE 1C; and/or a nucleotide sequence within no more than 10 kbp 5′ or no more than 10 kbp 3′ of a gene in a human genome selected from TABLE 1C;
wherein BLT-1 has a lower transposome density as compared to BLT-2;
wherein the first primer hybridizes to the first adaptor sequence and the second primer hybridizes to the second adaptor sequence.

115. The kit of claim 114, wherein BLT-2 has more than 10 times the transposome density as compared to BLT-1; and/or the first adaptor is B15 and the second adaptor is A14.

116. The kit of claim 114, comprising a plurality of at least 50 selection probes, wherein the selection probes are different from one another, wherein each selection probe of the plurality of selection probes comprises a nucleotide sequence having at least 90% sequence identity to any one of SEQ ID NOs:02-140601.

117. A system for preparing a nucleic acid library, comprising:
(a) a first plurality of transposomes comprising transposon adaptors for tagmenting a plurality of nucleic acid fragments, wherein the first plurality of transposomes is immobilized on a first plurality of beads at a first density;
(b) reagents for amplifying the plurality of polynucleotides to obtain amplified polynucleotides, wherein the amplifying comprising a mutagenesis PCR and/or a suppression PCR, wherein:
(i) the first reagent for performing mutagenesis PCR comprise a low bias DNA polymerase and/or a nucleotide analogue, and
(ii) the first reagents for performing suppression PCR comprise amplification primers having the same nucleotide sequence capable of hybridizing to the transposon adaptors;
(c) a plurality of selection probes for enriching for target polynucleotides in the amplified polynucleotides, wherein each selection probe of the plurality of selection probes comprises a nucleotide sequence capable of hybridizing to a gene selected from TABLE 1C; and/or a nucleotide sequence within no more than 10 kbp 5′ or no more than 10 kbp 3′ of a gene in a human genome selected from TABLE 1C; and
(d) a second plurality of transposomes comprising library adaptors for adding library adaptors to each end of the amplified polynucleotides, wherein the second plurality of transposomes is immobilized on a second plurality of beads at a second density, wherein the first density is less than the second density.

118. The system of claim 117, wherein the first plurality of the transposomes is immobilized on the first plurality of beads:
(i) at a density such that an average length of the plurality of polynucleotides is greater than about 1 kbp, and/or the average length of the plurality of polynucleotides is in a range from about 1 kbp to about 40 kbp;
(ii) such that the number of transposomes immobilized on the first plurality of beads is no more than about 100 transposomes per bead;
(iii) comprise a total activity such that an average length of the plurality of polynucleotides is greater than about 1 kbp, and/or wherein the average length of the plurality of polynucleotides is in a range from about 1 kbp to about 40 kbp; and/or
(iv) comprise an activity in a range from about 0.05 AU/μl to about 0.25 AU/μl.

119. The system of claim 117, wherein:
(i) an average distance between two adjacent nucleotide sequences of the selection probes on a reference sequence of a genome is in a range from about 300 consecutive nucleotides to about 7,000 consecutive nucleotides;
(ii) an average number of sites in a genome that each selection probe of the plurality of selection probes is capable of hybridizing to is no more than 50 different sites in the genome;
(iii) each selection probe of the plurality of selection probes is capable of hybridizing to no more than 50 different sites in a genome;
(iv) the plurality of selection probes lacks sequences capable of hybridizing to a repetitive genomic DNA element;
(v) the plurality of selection probes comprises at least 50 different selection probes; and/or
(vi) each selection probe of the plurality of selection probes comprises a nucleotide sequence having at least 90% sequence identity to any one of SEQ ID NOs:02-140601.
